SEQUENCE LISTING (82 sequences)

SEQ ID NO: 1 - DNA, 54 bp, Mus musculus
cggagtacagtgactttgagtttcagctattaaaatacttcttcatacgaaaaa

SEQ ID NO: 2 - DNA, 150 bp, Mus musculus
cggcaccctactggatcctggccaccgattatgaaaactatgccctcgtgtactcctgcaccaccttcttctggctcttccatgtggattttgtttggattcttggaagaaatccttatctccctccagaaacaataacctacctaaaaa

SEQ ID NO: 3 - DNA, 48 bp, Mus musculus
cggcatccagctggatgtcagagccaataaagatacatgcactaaaaa

SEQ ID NO: 4 - DNA, 55 bp, Mus musculus
cggccagagtctgattagggctttgctcttaagcaaaactgtttacagggaaaaa

SEQ ID NO: 5 - DNA, 121 bp, Mus musculus
cggccgtggcggacaacgagaaattggacaaccaacggctcaagaattttaagaacaaaggccgtgacttggagactatgagaagacaacgaaatgaagttgtagttgaattaaggaaaaa

SEQ ID NO: 6 - DNA, 166 bp, Mus musculus
cggcggtggccatcagactctgagacagagagccaatctcacattcaagtgttcaccaaccactgacgtgtttttatttccttctatatgattttaagatgtgttttctgcattctgtatagaaacatatcaaactaaataaaagcagtgtctttattaccaaaaa

SEQ ID NO: 7 - DNA, 343 bp, Mus musculus
cggggcaaggagcaccagaagaacagcagccaacggccaggagcgggcaccatggttctgctgcagcgggagctggctcaggaagacagcctcaacaagctggctctccagtatggctgcaaacactcagagtgattaacagtggcaaagtaagttggaaagcgtttctggaatcgctcttttctcccgcattgaaacagtctgtttcccgctgttgactccatgctatatacatgctatatacatgctgtatacatgctatatacatgctatatagtacatgctataatcatgctatatactggcagaagctttcaccaagcttgcatcagctacacaaaaa

SEQ ID NO: 8 - DNA, 138 bp, Mus musculus
cggtactccgctctgatcatcggcatggcatacggcgccaagcgctacactcaagatgacagcattctcaagtgaggcgtcagcgagcttgcttttctctagtcgttgagaacgaataaagcttcattgtgagaaaaa

SEQ ID NO: 9 - DNA, 39 bp, Mus musculus
cggtattcagtggtgatgcctaaaggaatgtcagaaaaa

SEQ ID NO: 10 - DNA, 45 bp, Mus musculus
cggtccctgccgctcaataaacatgaactgaacaaacaacaaaaa

SEQ ID NO: 11 - DNA, 56 bp, Mus musculus
cggtgcatttgttcaggtaaaatctgtgcaataaaataacaaactgtctccaaaaa

SEQ ID NO: 12 - DNA, 212 bp, Mus musculus
cggttgtggttcagtggcaaggcggttcagcacgtatccaacgtagatgagaccctaggttcagtctccatccagcactgggggctgggtgggatgtgacttagtctgtatgttgggaacaggaaaaaactccataaggtgagcaaaacagtattgttttcaattgaaatggttggttggttggttgttttgcttgtctaaagccgcaaaaa

SEQ ID NO: 13 - DNA, 1156 bp, Mus musculus
  misc_feature (670)..(670): n is a, c, g, or t
ttcggcagaggctcaatcgccaataaatgcatttccctgttaaatgaatggctaattaggtttatttttaccggtttggtgttgggccattgattttggtctcactaactagagtctccacttccctacaaattaggtagtttaaaaaataaccttccaggcctccgaggttaatttatattttaatgagtattaatagtcttcatgtcttcaagcattttcgctagagcatgtaaagtaaaacacatccaatttttcttgtcttgacatacacgtggagatgttaacgaaaagagattctgtatattttacctacttttctcccagccacttgttcaggttaatgagagatttttgaggtactaattgctttttatagacaaaccttttaactttgtatatataaaatatacacgtactcttggtgttcttttacaaaagctattaagtaggtgtaactaatactatgaagtagtttttttaaactagcttttaaaaggtaaggccttttcagtgtggatgcagcatggtgagtgatgattgtggatgcagttaactttgaaaatttgggtccctgtacctttgtgacaggttgttttaaaatagagactaatatttcaacttaatttcaaatgtattctgaaaaacttattatattagaaagtatgtntaaattcatttttaaaatgggggggtgggagatgccccatggactaagcattttttgcctttgcggagaacctaagttcggttccaccatccacatcaggtagctaaaaaccaccagaccctcggggctccacagacccacacatacatgtaattaaaagtgaaatgtgactgaaaacttgctaggaagtttctttggatcaaatagtctttgaaatgtataggcttccagtttaacatggtatgccctcttttgggtaccctttaaggaatagaagccggttgtggttcagtggcaagtcggttcagcacgtatccaacgtagatgagaccctaggttcagtctccatccagcactgggggctgggtgggatgtgacttagtctgtttgttgggaacaggaaaaaactccataaggtgagcaaaacagtattgttttcaattgaaatggttggttggttggttgttttgcttgtctaaagccact

SEQ ID NO: 14 - DNA, 118 bp, Mus musculus
cggcacttgggaggcagagacaggtggatgtctgagtttagagccagcctggtctacagagtgaattccagtctaggaaggtctacatagagaaatcctgtctcaaacaaaacaaaaa

SEQ ID NO: 15 - DNA, 122 bp, Mus musculus
cggctagcagcagaaacgtctcagggacagcacatgggcacagacgagttggacgggctgctctgcggggagaccaacggcaaagacacagagagttctgggtgctggggcaagaagaaaaa

SEQ ID NO: 16 - DNA, 240 bp, Mus musculus
cgggctagaacgccagccagaagaagcgctcgatctcggtctagaacgccagccaggagagggaggtcacgatccagaacaccagcacgacgacgatctcgaagtagaagtcttgtgagacgtggaagatctcactctagaacaccacaaagaagaggacgatctggctcatcctcagagaggaagaacaaatctagaacatctcagaggagaagcagatccaactcaagcccagaaaaa

SEQ ID NO: 17 - DNA, 220 bp, Mus musculus
cggtacgatgctgtgacaattaagattgatcctgaattggagaaaaaattgaaagtgaataaaataactttagagtcagagtatgagaggctgttatgtttattgtgcagacaatgataatccaccagagaagtattgccacaagcaagccgtccaagtacaatcacagacagcgactctacacaaggaacagagaatgaagtcagagggcacacaaaaa

SEQ ID NO: 18 - DNA, 299 bp, Mus musculus
cggtcatcgcagctgtcaatggttatgctcttggtgggggttgtgaacttgccatgatgtgtgatatcatctatgctggcgagaaagcccagttcggacagccagaaatcctcctggggaccatcccaggtgctggaggcactcagagactcacccgagcagtcggcaaatcgctagcaatggagatggtcctcactggtgaccgcatctcagctcaggatgcaaagcaggcaggtcttgtaagcaagatttttcctgttgaaaaactggttgaagaagccatccaatgtgcagaaaaa

SEQ ID NO: 19 - DNA, 279 bp, Mus musculus
cggtgacagacagaagaggattgtacagaggagctctttgacttcttgcatgcacgggaccactgtgtggcccacaagctccttaaaaacttgaagtaaatgtgcagattcgtcctcctcagccctgtttttgggaatcaggggcgagttccttgtggttctggacgtcggtgtctgatggagtgagttctcgagaacatcactgactccggcggtagcttctcttctgtgtgactagcagtgacttcatcttaataaactgatctgcaaacccaaaaa

SEQ ID NO: 20 - DNA, 48 bp, Artificial Sequence
  synthetic construct
  misc_feature (47)..(48): n is a, c, g, or t
gaattcaactggaagcggccgcaggaattttttttttttttttttvnn

SEQ ID NO: 21 - DNA, 16 bp, artificial sequence
  synthetic construct
aggtcgacggtatcgg

SEQ ID NO: 22 - DNA, 16 bp, artificial sequence
  synthetic construct
  misc_feature (16)..(16): n is a, c, g, or t
ggtcgacggtatcggn

SEQ ID NO: 23 - DNA, 15 bp, artificial sequence
  synthetic construct
gagctccaccgcggt

SEQ ID NO: 24 - DNA, 16 bp, artificial sequence
  synthetic construct
  misc_feature (13)..(16): n is a, c, g, or t
cgacggtatcggnnnn

SEQ ID NO: 25 - DNA, 16 bp, artificial sequence
  synthetic construct
cgacggtatcggcacc

SEQ ID NO: 26 - DNA, 16 bp, artificial sequence
  synthetic construct
cgacggtatcggttgt

SEQ ID NO: 27 - DNA, 16 bp, artificial sequence
  synthetic construct
cgacggtatcggtatt

SEQ ID NO: 28 - DNA, 155 bp, Mus musculus
cggctcagcacttggcagctgtcccgtgcggggactcagtccaactctgtgtttgcttttcttcttggccaaagcatgtgccactaagctgtcctggaggacattgtctttatgaaacacacctggaataaaaccacttcttacatgtccaaaaa

SEQ ID NO: 29 - DNA, 80 bp, Mus musculus
cggggctggagtaggtggcggaaggacatggacactgtctatctctgctctgttgtaataaatgtgagatcttggaaaaa

SEQ ID NO: 30 - DNA, 206 bp, Mus musculus
cggggtagggacacccctgtatcatagtggaggttggagctggcaaatgggaagagcttctaataatcactttgggctgggaaccttatttattggtagtgttaggtcagagggcagkakgcggagacaaggttgtggcacctgctgatgcagctcctctttattttgctttttacttgggaaataaatggatttagccataaaaa

SEQ ID NO: 31 - DNA, 206 bp, Mus musculus
cgggatcccacgagggccaccagcccaggggctcctgcccacccgcccttgggactaaaattcggctttgtaggagcgggtttggggaagtctggatagagactggacaaaggagtgtggccacagtgagaagtggatagcgccacagctgcggcgatggactcgttcataggaataaaatcttgctaacagcaaatgagcaaaaa

SEQ ID NO: 32 - DNA, 537 bp, Mus musculus
  misc_feature (525)..(525): n is a, c, g, or t
cagtttccctgcacctctggctctcaccagcatcctctcagctgttctgtgccaaatacgtctgcacccatgggttcagcacaggggcccctcacctcggtgactaagcttcgcccaccttgttacgatgtcttatatatatacacactgctatttacagacctggccttggatcctgtgaccctctgggaagagtcctgccaaagtccaggacagcattggggctaaggcagaggcttttctctagaggcttgggccctgtcccaacgtggactttggggctaggaacctgggcttctctctgtgaatgtaaggacagctactcagagagccttgatgggggcctctccccatttcctgtggtcagacccggctcagcacttggcagctgtcccgtgcggggactcagtccaactctgtgtttgcttttcttcttggccaaagcatgtgccactaagctgtcctggaggacattgtctttatgaaacacacctggaataaaaccacttcttacatgtccaaaanaaaaaaaaaaaa

SEQ ID NO: 33 - DNA, 2833 bp, Mus musculus
agctggtgtccggcggggccgtggctgctcctccacgcgtagccccgcacctgctgccccagtccagcccggagctccgcggccatgcggctggcgctgctctgggctctgggactcctgggcgcgggcagccctcggccctccccgccgctgccaaatataggaggcactgaggaagagcagcaagccagcccagagaggacgctgagtggatccatggagagccgggttgttcaggacagccccccaatgagcctagcagacgtgcttcagactggtttacctgaggccctgaggatttccttggagctggacagtgagagtcatgtcctggagcttctacaaaatagagatctaatccctggccgcccaactctggtgtggtaccagcctgatggcacccgaatggtcagcgagggctacagtctagaaaactgctgctaccgaggacgagtgcagggccaccccagctcctgggtgtccctctgtgcctgctctgggatcagggggctcattgtcctgtccccagagagaggctatacactggagctgggccctggggaccttcagcgtcctgtcatttctcggatccaagaccacctgttgctgggccacacctgtgccccaagctggcatgcctctgtgcccactcgggcaggaccagacctccttctggaacagcatcacgctcacaggcttaagcgagatgtagtaacagagacgaaaattgtggagttggtgattgtggctgataattcagaggtcagaaagtaccctgacttccaacaactgctgaaccggacactagaagcggctctcttgctagacacgttcttccagcccttgaatgtccgggtagcccttgtgggcctagaggcatggacccagcacaacctgatagaaatgagctccaacccagctgtcctgctagacaacttcctccgctggcgccggacagacttgctgcctcgactgccccatgacagtgcccaactggtgactgtaacttccttctctggtcccatggtgggcatggccattcagaattccatctgttcccctgacttctccggaggtgtgaatatggaccactccacaagcatcttaggcgttgcctcctcgattgcccatgaattgggccacagtctgggtttggaccatgattctcccgggcacagctgtccctgtccaggtccagccccggctaagagctgcatcatggaggcctccacagacttcctaccaggtttgaacttcagcaactgcagccgacaggccctggaaaaggccctcctggaaggaatgggcagctgcctcttcgaacggcaacccagcctggcccctatgtcctctttgtgtggaaatatgtttgtggaccccggagagcagtgtgactgtggcttcccagatgaatgcactgatccctgctgtgaccatttcacctgccagctgaggccaggagcgcagtgtgcatctgatggaccctgttgtcaaaactgcaagttgcacccagctggttggctgtgccgccctcccacagacgattgtgatctgcctgagttctgcccaggagatagctctcagtgcccgtctgacatcagacttggggacggtgagccttgtgctagtggagaggctgtgtgtatgcatgggcgctgtgcctcctatgcccggcagtgccagtcactttggggacccggggcccagcctgctgcgccactttgcctccaaacagccaacactcggggtaatgcctttgggagctgtgggcgcagccctggtggtagctacatgccttgtgcccctagagatgtcatgtgtgggcaactgcagtgccagtggggtaggagccagcctctgttgggctcagtccaagatcggctctcggaggtcctggaagccaacgggacacagttaaactgcagctgggtggacctggacctgggcaatgatgtggcccagcctcttctggctctgcctggcactgcctgtggtcctggcctggtgtgcatcggccaccgatgccagcccgtggatctcctgggagcacaggaatgtcgaagaaaatgccacggccatggggtctgtgacagcagcgggcactgccgctgtgaagagggctgggcacctccagactgcatgacccagctcaaagcaaccagctccctgaccacaggcctgctcctcagcctcctgttgttattggtcctcgtactacttggtgccagctactggcaccgtgcccgcctgcatcagcggctctgccagcttaagggatccagctgccaatatagggcaccccaatcctgtcctcctgaacgaccaggacctccacagcgggcacagcagatgacaggcactaagtctcaggggcctaccaaacccccacccccaagaaagccactgcctgccaacccacagggccagcacccaccaggtgacctgcctggcccaggagatggaagcttgccgctggtggtgccctccaggccagctccaccaccccctgcagcatcttcgctctacctctgacctctggagatttggctgcctccttctcaagctctaagactcaaagaaatggaacctctgccccaaacactagagaagcaggagaacagacaatctggtgtccagccctaaagaaccaccaggcgctgttaagcaatacctggggacgcactaaaatagctgcagcgggatgctggggaggggccgaagccggggctggagtaggtggcggaaggacatggacactgtctatctctgctctgttgtaataaatgtgagatcttggaaaaaaaaaaaaaaaaaaaaaaaaaaaaaaaaaaaa

SEQ ID NO: 34 - DNA, 596 bp, Mus musculus
  misc_feature (59)..(59): n is a, c, g, or t
  misc_feature (86)..(86): n is a, c, g, or t
  misc_feature (96)..(96): n is a, c, g, or t
tggcagtgctaagcagcactctaccagtgaatttacccccacactccctgcctttttcntttgtgtggttgaatcctggggatggnaacccagggnacagcagtccccagatcaactcccatcttctcagaggcactttagggcmrtggggctgggcagcacttcatgggtcctcaggcagttggggctaactgcctcaggaaggcatcccactttggagggcttccatctttttgaggcactttgggacagggaaagtgggtaccattctctcaggccttatgacaattggggtaactacgccaagcaggacagaggctgctggggcagggtggccttcccctcccccggtgtacatattgtacctgtgtactattttgtatataccggggtagggacacccctgtatcatagtggaggttggagctggcaaatgggaagagcttctaataatcactttgggctgggaaccttatttattggtagtgttaggtcagagggcagkakgcggagacaaggttgtggcacctgctgatgcagctcctctttattttgctttttacttgggaaataaatggatttagccataaaaaaaa

SEQ ID NO: 35 - DNA, 1603 bp, Mus musculus
cctcctcttacttctttttctccttctacttctcctcttctttcttctcctctttttcttcttcctcctcctccctctcctcccccatccccctgccccattgatgtgttattattgggggggctggagcagtaaaaaaagaaggaggaaaaaaagagcggggctcggcagggagagcttgagcgcgaggttgaccggcggcggcagcggccgcgatggaagaacttacggcgttcgtctccaagtcttttgaccagaaagtgaaggagaagaaggaggccatcacgtaccgggaggtgctagagagcgggccgctgcgcggggccaaagagcccggttgcgtcgagccgggccgcgacgaccgcagcagcccggcagtccgggcggccggcggaggcggcggcgcgggaggaggcggaggcggaggcggaggaggcggaggaggtgctggaggaggaggagcaggcggaggagctggaggagggcgctctcccgtccgggagctggacatgggagccgcggagcggagcagggagcccggcagcccgcggctgacggaggtgtcccctgaactgaaggatcgcaaagacgatgcgaaagggatggaggacgaaggccagaccaaaatcaagcagaggcgaagtcggaccaattttaccctggaacaactcaacgagctggagaggcttttcgatgagacccactatccagacgctttcatgcgcgaggaattgagccagcgactggggctctctgaggcccgagtacaggtttggtttcaaaatcgaagagctaagtgtagaaaacaggaaaatcaacttcacaaaggtgtccttataggagccgctagccagtttgaagcttgtagagttgcaccctatgtcaacgtaggtgctttaaggatgccatttcagcaggatagtcattgcaacgtgacgcccttgtcctttcaggttcaggcgcagctgcagctggacagcgccgtggcgcacgcgcaccaccacctgcatccgcacctggccgcgcacgcgccttacatgatgttcccggcaccgcccttcggactgccgctggccacgctggccgcggactcggcctcggccgcctcggtggtggccgctgccgccgccgccaagaccaccagcaagaactccagcatcgcggatctcagactgaaagctaaaaagcacgcggccgccctgggtctgtgacgccggcgccagcgccacggtcggtggagcctcctaagcggcgcgatcctgcacgccctccgcgaccggcttctcccgcacccgcttctgaccgtcgcccaggcctgtcccttccccgctgactgccgccttttctttctgcaccctggatccccagggcgggactctgcgctggacccgggatcccacgagggccaccagcccaggggctcctgcccacccgcccttgggactaaaattcggctttgtaggagcgggtttggggaagtctggatagagactggacaaaggagtgtggccacagtgagaagtggatagcgccacagctgcggcgatggactcgttcataggaataaaatcttgctaacagcaaatgagcaaaaaaaaaaaaa

SEQ ID NO: 36 - DNA, 271 bp, Mus musculus
cggctgcaggtgagggctggtttgtaacgaattctctctgccctcttaagctgaggaagctggagtaggtctcatttgccctgtagttgcgatctctgatggctggggagcatctttcctcatgtttgctgtgtatctgcttcagagacttcagggtgtttgcccawwrrgttgtctgaccttttattatgaaggtttacaagtttgttatgcattctagataaaagttcctttgtgtcagatgaatcacataaaaattttcctccaaaaa

SEQ ID NO: 37 - DNA, 411 bp, Mus musculus
cggctaatattgataatctttatttgaaaaaatgtcatgaaccatttgaatgatgagccacagaacctcagttgaatttatttccacttttggcatgttaaatatagatttaattttaagtacttcaattaatgggtttataaagtcaagcactagcattggtcagttttgtatgataggatgtaagtgtgttctcacctgcagtgtaaatacagcacactgtagaattctcttaaggtgcatagtaaatgtatagatagtcacaggcggttttgtaatgtatacatttctaatctattattcctaacctgtcatgtttgcagagagaaaagaatttttctaatgatctgtaaaattatgttaacttctacaagtaggtattctaaataaacttttttaaaagaccaaaaa

SEQ ID NO: 38 - DNA, 295 bp, Mus musculus
cggacggtgtaccccgaggatcgccccaggtggagggaaagatccaggaccaggtcgcgcagcaggagtagaaccccatttcgcctgtgtgagaaagatcgaatggagctactagaaatagcaaaagccaacgcagcaaaagctctgggaacagccaacttcgacttgccagcaagtctccgagccaaggaggcaagccaggggacagctgtttccagcagtgggccaaaggtggagcattcagaaaagcagactgaagatacaactaaaaataccagtgaaaagtcttctacac

SEQ ID NO: 39 - DNA, 84 bp, Mus musculus
cggaatactgaggaggaaggacccaagtacaagtccaaagtttcattaaaaggcaatagagaaagtgatggatttagagaaaaa

SEQ ID NO: 40 - DNA, 42 bp, Mus musculus
cggccggcatgaaataaaacatttaaatagtgctggcaaaaa

SEQ ID NO: 41 - DNA, 397 bp, Mus musculus
cggctgacaacagactttaatgtaattgtgcaagcactgagcaaatctaaggcaaaactcatggaagtcagtgcagacaaaactaaaattagaagatcaccaagcagaccactccctgaagtgacggatgagtataagaatgatgtaaaaaacagatctgtttatattaaaggtttcccaactgacgccacccttgatgatataaaagaatggctagacgataaaggccaaatactgaatattcaaatgagaagaacattacacaaaacatttaaggggtcaatatttgctgtgtttgatagtattcagtctgcaaagaagtttgtggagatccctggccagaagtacaaagacactaacctgctaatactctttaaggaagattactttgcaaaaa

SEQ ID NO: 42 - DNA, 240 bp, Mus musculus
cggccgtggtggcgcacaccattaatcccagcactcaggaggcagaggcaggcggatttctgagttcgaggccagcctggtctacagagtgagttccaggacagccagggctacacagagaaaccctgtcttgaagaaacaaaaaggttaggctagtatttggagaaagaagattagaaaatggaagtgaaagacgaagaagacatacaggaaggtgaagaaaaagctgttagagaaaaa

SEQ ID NO: 43 - DNA, 196 bp, Mus musculus
cggcatgggtggtcttcatcctggccgatagctgcagaactgatgtgaatgtaccttcatttgctctgacactgcatggcacagtggcaggattgcacatccctagagtagaggctttcaagcaaagctgcctcccccgtcttgatttcctgttgatttctattctataattgaacaggcatttctgtggcaaaaa

SEQ ID NO: 44 - DNA, 95 bp, Mus musculus
cggcagacctagctcagcttgatggggtgtgacaactgcaattagaggcaagccgcctgctgcccccagagcattaagagcaaattggagaaaaa

SEQ ID NO: 45 - DNA, 273 bp, Mus musculus
cgggccccatcaatttcaccatgttcctcaccatgtttggggagaagctaaacggcactgaccccgaggacgtcatcagaaacgccttcgcttgctttgatgaggaagccacaggcaccatccaggaggattacctgagggagcccctgaccaccatgggcgaccgcttcacagacgaggaagtggatgagctgtacagagaggcccccattgacaaaaaggggaacttcaactacattgagttcacacgcatcctgaagcacggcgcaaaaa

SEQ ID NO: 46 - DNA, 273 bp, Mus musculus
cgggcgccatcaatttcaccatgttcctcaccatgtttggggagaagctaaacggcactgaccccgaggacgtcatcagaaacgccttcgcttgctttgatgaggaagccacaggcaccatccaggaggattacctgagggagctgctgaccaccatgggcgaccgcttcacagacgaggaagtggatgagctgtacagagaggcccccattgacgaaaaggggaacttcaactacattgagttcacacgcatcctgaagcacggcgcaaaaa

SEQ ID NO: 47 - DNA, 115 bp, Mus musculus
cggggctcaaagacaagggttcgagtcccgctcctgcccacgcccactgcattcgggcttcagtttttccttctctgaaatggggacgtggataaaatcatcttcaaagcaaaaa

SEQ ID NO: 48 - DNA, 335 bp, Mus musculus
cggtcatcgcagctgtcaatggttatgctcttggtgggggttgtgaacttgccatgatgtgtgatatcatctatgctggcgagaaagcccagttcggacagccagaaatcctcctggggaccatcccaggtgctggaggcactcagagactcacccgagcagtcggcaaatcgctagcaatggagatggtcctcactggtgaccacatctcagctcaggatgcaaagcaggcaggtcttgtaagcaagatttttcctgttgaaaaactggttgaagaagccatccaatgtgcagaaaaaattgccagcaattctaaagtcgtagtagccatggcg

SEQ ID NO: 49 - DNA, 240 bp, Mus musculus
cggtatgtgggtagagtggtccattcgtttgatggcacgaaggaagcagcagctgctttggttgacttgggcctttatataggatttaatggttgctctctgaaaactgaagctaacttggaagttctgaagtcaatacctagtgaaaaactaatgattgagacagatgcaccttggtgtggagttaaaagtacacatgctggatcaaaatacataaacccttgggtttccctccaaaaa

SEQ ID NO: 50 - DNA, 107 bp, Mus musculus
cggtatccacagtaaaattgtgagtagcttaatctgtttatctccattacaattcctctgcaactattttccttgatgttgtaataaaaaggaggtaggatgaaaaa

SEQ ID NO: 51 - DNA, 21 bp, artificial sequence
  synthetic construct
aacaagtccgtcctggcatgg

SEQ ID NO: 52 - DNA, 59 bp, artificial sequence
  synthetic construct
tcccagtcacgacgttgtaaaacgacggctcatatgaattaggtgaccgacggtatcgg

SEQ ID NO: 53 - DNA, 46 bp, artificial sequence
  synthetic construct
cagcggataacaatttcacacagggagctccaccgcggtggcggcc

SEQ ID NO: 54 - DNA, 23 bp, artificial sequence
  synthetic construct
cccagtcacgacgttgtaaaacg

SEQ ID NO: 55 - DNA, 19 bp, artificial sequence
  synthetic construct
ttttttttttttttttttv

SEQ ID NO: 56 - DNA, 35 bp, artificial sequence
  synthetic construct
ggtggcggccgcaggaatttttttttttttttttt

SEQ ID NO: 57 - DNA, 16 bp, artificial sequence
  synthetic construct
cgacggtatcggagta

SEQ ID NO: 58 - DNA, 16 bp, artificial sequence
  synthetic construct
cgacggtatcggacgg

SEQ ID NO: 59 - DNA, 16 bp, artificial sequence
  synthetic construct
cgacggtatcggtgca

SEQ ID NO: 60 - DNA, 16 bp, artificial sequence
  synthetic construct
cgacggtatcggctag

SEQ ID NO: 61 - DNA, 16 bp, artificial sequence
  synthetic construct
cgacggtatcggctca

SEQ ID NO: 62 - DNA, 16 bp, artificial sequence
  synthetic construct
cgacggtatcggggca

SEQ ID NO: 63 - DNA, 16 bp, artificial sequence
  synthetic construct
cgacggtatcggggct

SEQ ID NO: 64 - DNA, 16 bp, artificial sequence
  synthetic construct
cgacggtatcggggta

SEQ ID NO: 65 - DNA, 16 bp, artificial sequence
  synthetic construct
cgacggtatcggtatt

SEQ ID NO: 66 - DNA, 16 bp, artificial sequence
  synthetic construct
cgacggtatcggctaa

SEQ ID NO: 67 - DNA, 16 bp, artificial sequence
  synthetic construct
cgacggtatcggtcat

SEQ ID NO: 68 - DNA, 1717 bp, Homo sapiens
gatattttaaaaattgcatacatatgaaatatattgggatttacaaacaactaaacacatatactcacataaataccaatatattctattaaatttaaagtttacattttcttcaagcttgattttgaagtaggatattgcttgatgtcattgccaggtgcaaagttgcaaggaaacgtgagtttataactttgttattgccagtgcatcatagaagtaaatgtagtataaaataataggctataatatttttgtagtggatctcttgtatatattgttcactttgatgtctttttcagctacccttttttacctaagttttatagctatagattttattattattttttgtttccacatttaaagaatgcttggagtagtcctgagaagagttcatattttcaacattagctggcttgtttacatatctgtctgaaataaatataatgttttggtaattttcattaattgataaaggcaggtgaggcttctcaaacagaaactgtatctgaagaaaacaaaagccttatctggacactattgaaacaagtccgtcctggcatggacctatccaaggtggttctgcctacatttattttggaaccccgttctttcctggataaactttcagattactactatcatgcagatttcctatctgaggcagctcttgaagaaaatccttatttccgtttgaagaaagtagtgaaatggtatttgtcaggattctataaaaagccaaagggactgaagaaaccttataatcctatacttggcgagactttccgttgtttatggattcatcccagaacaaacagcaaaactttttatattgctgaacaggtgtcccatcatccaccaatatctgccttttatgttagtaatcgaaaagatggattttgccttagcggtagtatcctggctaagtctaagttttatggaaactcattatctgcaatattagagggagaagcacggttaactttcttgaatagaggtgaagattatgtaatgacaatgccatacgctcattgtaaaggaattctttatggtacaatgacactggagcttggtggaacagtcaatattacatgtcaaaaaactggatacagtgcaatacttgaatttaaactaaagccattcctagggagtagtgactgtgttaatcaaatatcagggaaacttaaactgggaaaagaagtcctagctactttggaaggtcattgggatagtgaagtttttattactgataaaaagactgataattcagaggttttctggaatccaacacctgayattaagcaatggagattaataaggcacactgtaaaatttgaagaacagggagattttgaatcagagaaactctggcaacgggtaactcgagccataaatgccaaagaccaaactgaagctacccaagagaagtatgttttggaagaagctcaaagacaagctgccagggatcggaaaacaaaaaatgaagagtggtcttgcaaattatttgaacttgatccactcacaggagaatggcattacaagtttgcagatacccgaccatgggacccacttaatgatatgatacagtttgaaaaagatggtgttattcagaccaaagtgaaacatcgtactccaatggttagcgtccccaaaatgaaacataagccaaccaggcaacagaagaaagtagcaaaaggctattcctccccagaaccgg

SEQ ID NO: 69 - DNA, 1717 bp, Homo sapiens; identical to SEQ ID NO: 68

SEQ ID NO: 70 - protein, 385 aa, Homo sapiens
MDLSKVVLPTFILEPRSFLDKLSDYYYHADFLSEAALEENPYFRLKKVVKWYLSGFYKKPKGLKKPYNPILGETFRCLWIHPRTNSKTFYIAEQVSHHPPISAFYVSNRKDGFCLSGSILAKSKFYGNSLSAILEGEARLTFLNRGEDYVMTMPYAHCKGILYGTMTLELGGTVNITCQKTGYSAILEFKLKPFLGSSDCVNQISGKLKLGKEVLATLEGHWDSEVFITDKKTDNSEVFWNPTPDIKQWRLIRHTVKFEEQGDFESEKLWQRVTRAINAKDQTEATQEKYVLEEAQRQAARDRKTKNEEWSCKLFELDPLTGEWHYKFADTRPWDPLNDMIQFEKDGVIQTKVKHRTPMVSVPKMKHKPTRQQKKVAKGYSSPEP

SEQ ID NO: 71 - protein, 472 aa, Homo sapiens
MDLSKVVLPTFILEPRSFLDKLSDYYYHADFLSEAALEENPYFRLKKVVKWYLSGFYKKPKGLKKPYNPILGETFRCLWIHPRTNSKTFYIAEQVSHHPPISAFYVSNRKDGFCLSGSILAKSKFYGNSLSAILEGEARLTFLNRGEDYVMTMPYAHCKGILYGTMTLELGGTVNITCQKTGYSAILEFKLKPFLGSSDCVNQISGKLKLGKEVLATLEGHWDSEVFITDKKTDNSEVFWNPTPDIKQWRLIRHTVKFEEQGDFESEKLWQRVTRAINAKDQTEATQEKYVLEEAQRQAARDRKTKNEEWSCKLFELDPLTGEWHYKFADTRPWDPLNDMIQFEKDGVIQTKVKHRTPMVSVPKMKHKPTRQQKKVAKGYSSPEPDIQDSSGSEAQSVKPSTRRKKGIELGDIQSSIESIKQTQEEIKRNIMALRNHLVSSTPATDYFLQQKDYFIIFLLILLQVIINFMFK

SEQ ID NO: 72 - DNA, 30 bp, artificial sequence
  synthetic construct
gatcgaatccggctaatattgataatcttt

SEQ ID NO: 73 - DNA, 20 bp, artificial sequence
  synthetic construct
ggatcctggccaccgattat

SEQ ID NO: 74 - DNA, 20 bp, artificial sequence
  synthetic costruct
tggtgcaggagtacacgagg

SEQ ID NO: 75 - DNA, 21 bp, artificial sequence
  synthetic construct
ggttcagcacgtatccaacgt

SEQ ID NO: 76 - DNA, 21 bp, artificial sequence
  synthetic construct
tgctggatggagactgaacct

SEQ ID NO: 77 - DNA, 22 bp, artificial sequence
  synthetic construct
aatgatgagccacagaacctca

SEQ ID NO: 78 - DNA, 25 bp, artificial sequence
  synthetic construct
aacatgccaaaagtggaaataaatt

SEQ ID NO: 79 - DNA, 336 bp, Mus musculus
aaacatcgtactccaatggttagcgttccaaaaatgaaacataaaccaacaaggcagcagaagaaagtggtaaaaggctattcttccccagaacctgacatccaggattcctctggaagtgaagctcaatctgtaaaaccaagtacacgaaggaagaaggggatagacctgggagacattcagagttccattgagtctataaagcagacacaggaagagattaaaagaaatattatggctcttcgaaatcatttactttcaagtacacctgctacagattattttctgcaacagaaagactacttcgtcattttcctcctgattttgcttcaagtc

SEQ ID NO: 80 - DNA, 652 bp, Mus musculus
cttgagcatggtgagtgagtgattgyggatgcagttaactttgaaaatttgggtccctgtacctttgtgacaggttggttctaaaatagagactaatatttcaacttaattkcaaatgtgattctgaaaaacttattatattagaaagtatgtttaaattcatttttaaaactaaaatgggggggtgggagatgccccatggactaagcattttttgcctttgcggagaacctaagttcaggttccaccatccacatcaggcasctaaaaaccaccagaccctcggggctccacagacccacacatacatgtaattaaaagtgaaatgtgactgaaaacttgctaggaagtttctttggatcaaatagtctttgaaatgtataggcttccagtttaacatggtatgccctcttttgggtaccctttaaggaaatagaagccggttgtggttcagtggcaagtcggttcagcacgtatccaacgtagatgagaccctaggttcagtctccatccagcactgggggctgggtgggatgtgacttagtctgtttgttgggaacaggaaaaaactccataaggtgagcaaaacagtattgttttcaattgaaatggttggttggttggttgttttgcttgtctaaagccactaaaa

SEQ ID NO: 81 - DNA, 21 bp, artificial sequence
  synthetic construct
ccaatggttagcgttccaaaa

SEQ ID NO: 82 - DNA, 21 bp, artificial sequence
  synthetic construct
cttctgctgccttgttggttt

(19) United States
(12) Patent Application Publication
(10) Pub. No.: US 2007/0010664 A1
(43) Pub. Date: Jan. 11, 2007

(54) Gene expression in the central nervous system regulated by neuroleptic agents

(76) Inventors: Elizabeth A. Thomas, Encinitas, CA (US); J. Gregor Sutcliffe, Tiempo Cardiff-by-the-Sea, CA (US); Thomas M. Pribyl, San Diego, CA (US); Brian S. Hilbush, San Diego, CA (US); Karl W. Hasel, Nardo Avenue Solana Beach, CA (US)

Correspondence Address: DAVID R PRESTON & ASSOCIATES APC, 5850 OBERLIN DRIVE, SUITE 300, SAN DIEGO, CA, 92121, US

(21) Appl. No.: 10/381,957

(22) PCT Filed: Oct. 1, 2001
(86) PCT No.: PCT/US01/30695

§ 371 Date: Oct. 6, 2003

(51) Int. Cl. C07H 21/04

(52) U.S. Cl. 536/23.2

(57) ABSTRACT

Polynucleotides, polypeptides, kits and methods are provided related to genes expressed in the central nervous system that are regulated by neuroleptics.

### RELATED APPLICATION

The present application claims priority to U.S. Provisional Patent Application No. 60/236,790, filed Sep. 29, 2000, and U.S. Provisional Patent Application No. 60/263,084, filed Jan. 18, 2001 both of which are hereby incorporated by reference.

### BACKGROUND OF THE INVENTION

Neuropsychiatric disorders, including schizophrenia, affective and behavioral disorders, are a heterogeneous group of devastating illnesses that can impair all aspects of a patient's life. Although positive symptoms, such as hallucinations and delusions are often emphasized, the negative symptoms of these disorders prevent patients from functioning in society, maintaining a job or exhibiting proper social behavior. Mental disorders, such as schizophrenia, represent a major public health problem that affects not only the patients and families, but imposes a costly impact on the health system and economy as well (Wasylenki, D. A., Can. J. Psych., 39:S35 (1994); Miller, D. D., Pharmacotherapy, 16: 2 (1996)). It has been estimated that approximately 30-50% of homeless Americans have some form of mental illness (Susser et al., Community Ment. Health J., 26: 391 (1990)). Genetic studies have implicated several susceptibility loci for schizophrenia on five distinct chromosomes; however, the etiology and pathophysiology of the disease have yet to be determined. Given the heterogeneity of the disease, it is not surprising that no single biological system or anatomical region has been proven to be pivotal to pathology. It is thought that dysfunction in multiple brain regions contribute to the overall manifestation of disease symptoms and numerous reports have identified abnormalities throughout the brain; however, there is still no absolute consensus regarding which brain regions and neurochemical systems are most affected.

Given their apparent function in normal and diseased brain states, it is likely that midbrain dopamine neurons play an important role in the development of neuropathology. For example, many psychiatric disorders are associated with overactive dopaminergic activity in the meso-striatal dopamine system which refers to both the nigro-striatal dopamine pathway (neurons linking the substantia nigra to the striatum), and the meso-limbic dopamine pathway (neurons linking the ventral tegmental area to limbic regions, such as amygdala, olfactory tubercle and the nucleus accumbens, which is often considered a ventral extension of the striatum). Additionally, it is known that Parkinson's Disease is caused by the degeneration of dopamine neurons of the nigro-striatal pathway.

In the general population, the risk for developing a psychiatric disorder is approximately 1% (Maier et al., Curr. Opin. in Psych., 11: 19 (1998); Kendler et al., Arch. Gen. Psych., 50: 9095 (1993)). However, this risk increases to 10% or 40% if one or both parents, respectively, have the disease. Concordance in monozygotic and dizygotic twins remains only as high 40-50% (Kendler et al., supra (1993)). While there is undoubtedly a genetic component to the transmission of psychiatric disorders, the lack of fall concordance in dizygotic twins indicates that there are other environmental factors that contribute (Maier et al., supra (1998); Kendler et al., supra (1993)). A current challenge in genetic research on mental illnesses is the identification of mutations conferring susceptibility to, or genes associated with therapeutics for, such disorders. One approach addressing the latter is to identify genes whose expression is altered during the process of drug treatment. Considerable evidence demonstrates that the ameliorative effects of neuroleptic drugs, as well as their unwanted motor side effects, are the result of changes in gene expression.

Examples of neuroleptic drugs that are widely used in the long-term treatment of various psychiatric disorders, such as schizophrenia, include haloperidol and clozapine. The antipsychotic effects of neuroleptic drugs are generally attributed to blockade of D₂ receptors in the meso-limbic dopamine system (Metzler et al., Schizophrenia Bull., 2, 19-76 (1976)). The best evidence for this comes from the excellent correlation observed between the therapeutic potency of neuroleptics and their affinity for binding to the D₂ receptor (Seeman et al., Curr. Opn. Neurol. And Neurosurg., 6, 602-608 (1993); Creese et al., Science, 192, 481-483 (1976); Peroutka et al., Am. J. Psych., 137, 1518-1522 (1980); Deutch, et al., Schizophren. Res., 4, 121-156 (1991); Seeman, P., Synapse 1, 133-152 (1987)). Although neuroleptic drugs have affinity for other neurotransmitter receptors in the brain, such as muscarinic acetylcholine, 5-HT, alpha-adrenergic and histamine receptors, no correlation to clinical efficacy has been observed with these receptors (Peroutka et al., Am. J. Psych. (1980); Richelson et al., Eur. J. Pharm., 103, 197-204 (1984)).

Human brain imaging studies have demonstrated that dopamine receptors become blocked to a level of >70% after only a few hours of treatment with various neuroleptic drugs (Sedvall et al., Arch. Gen. Psych., 43: 995 (1986)). This blockade has been shown to lead to a compensatory increase in dopamine receptor number and supersensitivity of the unblocked receptors (Clow et al., Psychopharm., 69, 227-233 (1980); Rupniak et al., Life Sci., 32, 2289-2311 (1983); Rogue et al., Eur. J Pharm., 207, 165-169 (1991)). Furthermore, the short-term effects of dopamine antagonists on the brain are well known and include such effects as an increase in dopamine synthesis and catabolism, an increase in the firing rate of dopamine neurons resulting from the inhibition of pre-synaptic dopamine autoreceptors (Grace et al., J. Pharm. Exp. Ther., 238, 1092-1100 (1986), and a potentiation of cyclic AMP formation resulting from the blockade of post-synaptic dopamine receptors (Rupniak et al., Psychopharm., 84, 519-521 (1984)).

In addition to their antipsychotic actions, neuroleptics can cause a series of mild to severe side effects. Some of these side-effects result from the non-specific nature of neuroleptic drugs, including hypotension and tachycardia, which results from alpha-adrenergic receptor blockade, and dry mouth and blurred vision, which results from the blockade of muscarinic acetylcholine receptors. The predominant and most undesirable effects that accompany neuroleptic treatment are the long-lasting motor deficits referred to as extrapyramidal side effects (Marsden et al., Psychol. Med., 10, 55-72 (1980)). Extrapyramidal side effects are associated with the blockade of dopamine receptors in the dorsal striatum (Moore et al., Clin. Neuropharmacol., 12, 167-184 (1989) and include such motor deficits as dystonias (muscle spasms), akathisias (motor restlessness), Parkinson's-like symptoms and Tardive Dyskinesia. Roughly 20% of patients taking antipsychotics demonstrate Parkinson's-like symptoms, the blockade of dopamine D₂ receptors in the striatum being functionally equivalent to the degeneration of nigro-striatal dopamine neurons seen in Parkinson's Disease. Tardive Dyskinesia is a syndrome of abnormal involuntary movements that afflicts roughly 25% of patients on neuroleptic treatment (Jeste et al., Psychopharmacol., 106, 154-160 (1992); Casey, D. E., Schizo. Res., 35: S61 (1999)). The danger of this side effect is that it can be potentially irreversible, that is, patients can still have symptoms of Tardive Dyskinesia long after the antipsychotic has been discontinued. This implicates an epigenetic component to the effects of chronic neuroleptic treatment.

Interestingly, “typical” neuroleptics, such as haloperidol and fluphenazine, have a much higher propensity for causing extrapyramidal side effects than “atypical” neuroleptic drugs, such as clozapine, which rarely causes these types of effects. Although clozapine differs from haloperidol in its pharmalogical profile, the specific mechanism leading to the lack of motor side effects is unclear. Since clozapine has high affinity for other neurotransmitter receptors, such as muscarinic, adrenergic and serotonin receptors, it is possible that the antipsychotic actions of clozapine are partly due to blockade of these other receptors, which may restore proper balance of the dopamine input and output pathways of the basal ganglia.

Despite the immediate occupancy of dopamine receptors, neuroleptic drugs have a delayed onset of clinical action, which often can be up to several weeks. Further, as discussed above, neuroleptic drugs are characterized by their ability to cause late and long-lasting motor deficits (Marsden et al., Psychol. Med., 10: 55 (1980)). The distinct temporal discrepancy which exists between dopamine receptor occupancy and the onset of therapeutic and extrapyramidal side effects, suggests that additional molecular changes in the brain occur downstream from dopamine receptor blockade. In an attempt to identify the downstream molecular mechanisms, studies have focused on dopamine-receptor regulation of individual target genes in the striatum and nucleus accumbens.

For example, several studies have demonstrated that acute treatment with antipsychotic drugs causes induction of several immediate-early genes (Hughes et al., Pharmacol. Rev., 47: 133 (1995); Fibiger, H. C., J. Clin. Psych., 55: 33 (1994); Nguyen et al, Proc. Natl. Acad. Sci., 89, 42704274 (1992); MacGibbon et al., Mol. Brain. Res. 23, 21-32 (1994); Robertson et al., Neuro. Sci., 46, 315-328 (1992); Dragunow et al., Neuro. Sci., 37, 287-294 (1990); Miller J., Neurochem., 54, 1453-1455 (1990); Rogue et al., Brain Res. Bull., 29: 469 (1992)). Some immediate early gene proteins (IEGPs) act as transcription factors by binding to specific DNA sequences and regulating gene transcription. Thus, IEGPs can link receptor-mediated signalling effects to long-term changes in genomic activity. Recent studies have shown that haloperidol, a typical neuroleptic, induces the expression c-Fos in the rat striatum and nucleus accumbens, whereas, clozapine, an atypical neuroleptic, induces c-Fos in the nucleus accumbens only (Nguyen et al., Proc. Natl. Acad. Sci. (1992); MacGibbon et al., Mol. Brain Res. (1994); Robertson et al., Neurosci. (1992)). Haloperidol has also been shown to induce expression of other IEGPs, such as FosB, JunB, JunD and Krox24, in the striatum and nucleus accumbens (Rogue et al., Brain Res. Bull. 29, 469472 (1992); Marsden et al., Psych. Med. (1980); Moore et al., Clin. Neuropharmacol. (1989)). In contrast, clozapine has been shown to induce Krox24 and JunB in the nucleus accumbens only (Nguyen et al. (1992); MacGibbon et al. (1994)). These results suggest that clozapine's lower tendency to cause extrapyramidal side effects, compared to “typical” neuroleptics, may be associated with its failure to induce IEGPs in the striatum.

The appearance of immediate early genes after acute treatment with neuroleptics likely precedes a number of other molecular changes responsible for the delayed adaptive changes that occur with drug treatment in the striatum.

Chronic treatment with neuroleptic drugs has also been shown to cause changes in the expression of certain neuropeptides and neurotransmitter receptors. In distinct regions of the striatum, both neurotensin and enkephalin are upregulated after chronic (7-28 days) treatment with haloperidol, while levels of protachykinin mRNA are decreased (Merchant et al., J. Pharm. Exp. Ther., 271, 460-471 (1994); Delfs et al., J. Neurochem., 63, 777-780 (1994); Angulo et al., Neurosci. Lett. 113, 217-221 (1990)). In contrast, chronic clozapine treatment results in a decrease in enkephalin mRNA levels and only small changes in the expression of neurotensin and tachykinin (Merchant et al. (1994); Mercugliano et al., Neurosci. Lett., 136, 10-15 (1992); Angulo et al. (1990)). These differences suggest that neuropeptides may play a role in the motor deficits that result from treatment with typical neuroleptics.

Researchers have also demonstrated the regulation of genes associated with glutaminergic neurotransmission. For example, a decrease in mRNA expression of the glutamate transporter, GLT-1, was observed in the striatum after 30 days of haloperidol treatment, but not after clozapine exposure (Schneider et al., Neuroreport., 9, 133-136 (1998)). Similar treatment with haloperidol also resulted in an increase in the N-methyl-D-aspartate (NMDA) receptor subunits, NR1 and NR2, whereas clozapine treatment resulted in a lesser induction (Riva et al., Mol. Brain. Res. 50, 136-142 (1997)).

In addition, pathological and structural changes in the striatum have been observed after chronic drug treatment. Studies using experimental animals have detected a reduction in the size and number of striatal neurons and neuronal processes, as well as decreases in striatal neuronal density following chronic treatment with haloperidol (Christensen et al., Acta. Psych. Scand., 46, 14-23 (1970), Jeste et al., Psychopharm., 106, 154-160 (1992); Mahadik et al., Biol. Psych., 24, 199-217 (1988); Nielson et. al., Psychopharm., 59-85-89 (1978). These studies imply that neuroleptics may have a neurotoxic effect on the striatum which could account for the ensuing neuroleptic-induced side effects.

Long-term changes in the expression of critical genes resulting from neuroleptic drug therapy may compensate for underlying genetically determined biochemical deficits, thereby restoring a state of normal mental activity, or alternatively, can cause detrimental or permanent consequences. Hence, genes that are regulated by drug treatment may provide information regarding pathways responsible for behavioral dysfunction. Although the above studies have examined the expression of a few individual target genes, there has been no comprehensive study of the effects of neuroleptics on gene expression over time in the striatum and nucleus accumbens, brain regions considered to be critically involved in the actions of neuroleptic drugs. Thus, the number and identity of the genes which are differentially expressed following acute and chronic treatment with neuroleptics in these tissues remains unknown. Further, there has been no comprehensive examination of the differences between the striatal mRNA expression induced by typical neuroleptics and the expression induced by atypical neuroleptics.

Such a systematic characterization would allow the identification of genes that contribute to neuropathologies associated with neuropsychiatric disorders. This information can reveal pathways for the mechanism of actions of antipsychotic drugs, as well as provide insight regarding the underlying basis of psychiatric dysfunction. Specifically, the identification of potentially harmful gene products is important to identify molecules that could be useful as diagnostic markers indicating neuropathology. Additionally, the identification of potentially harmful gene products is important to identify molecules that could be amenable to pharmaceutical intervention. A systematic characterization would also allow the identification of beneficial molecules that contribute to conditions of neuroprotection. Such identification of beneficial products could lead to the development of pharmaceutical agents useful in the treatment of neuropsychiatric disorders. Furthermore, the identification of harmful and beneficial products may lead to new lines of study towards the amelioration of symptoms associated with neuropsychiatric disorders.

Such a comparative study would also identify the genes that regulate the antipsychotic actions of neuroleptics versus those responsible for the unwanted side effects associated with these drugs. This information would advance the development of an antipsychotic therapy that would target specific actions of neuroleptic drugs or, alternatively, would selectively block proteins causing the motor side effects.

What is needed therefore, is a comprehensive examination of the differences between the striatal mRNA expression induced by typical neuroleptics and the expression induced by atypical neuroleptics. The Total Gene Expression Analysis (TOGA™) method, described in Sutcliffe et al., Proc. Natl. Acad. Sci. USA 97(5): 1976-81 (2000), International published application WO 00/26406, U.S. Pat. No. 5,459,037, U.S. Pat. No.5,807,680, U.S. Pat. No. 6,030,784, U.S. Pat. No. 6,096,503 and U.S. Pat. No. 6,110,680, all of which are incorporated herein by reference, is a tool used to identify and analyze mRNA expression. The TOGA™ method is an improved method for the simultaneous sequence-specific identification of mRNAs in an mRNA population which allows the visualization of nearly every mRNA expressed by a tissue as a distinct band on a gel whose intensity corresponds roughly to the concentration of the mRNA. The method can identify changes in expression of mRNA induced by typical neuroleptics and the expression induced by atypical neuroleptics.

### SUMMARY OF THE INVENTION

The present invention provides polynucleotides and the encoded polypeptides that are regulated by neuroleptic use. The present invention also provides different uses of these polynucleotides and polypeptides. The invention was made while performing studies using the PCR-based Total Gene Expression Analysis (TOGA™) method to analyze the expression patterns of thousands of genes and comparing the expression patterns among time courses following clozapine drug treatment. Genes regulated by clozapine treatment were examined in haloperidol-treated animals for a comparative analysis. TOGA™ analysis identified several genes that were altered in their expression in response to clozapine and/or haloperidol administration in mouse brain. In particular, the TOGA™ system was used to examine how gene expression in the striatum and nucleus accumbens is regulated by an atypical neuroleptic agent, such as clozapine. These studies identified proteins and genes which are regulated by the treatment of atypical drugs. Further, these studies identified at least one gene which is differentially regulated by typical and atypical drugs.

The studies also examined the pattern of expression of neuroleptic-regulated genes in various regions of the brain. Among other things, these studies were used to determine the genes specifically associated with anti-psychotic activity versus those associated with extrapyramidal side effects, which information advances the development of improved antipsychotic therapies. The identified neuroleptic-regulated molecules are useful in therapeutic and diagnostic applications in the treatment of various psychiatric disorders, such as psychoses and addiction-related behavior. Such molecules are also useful as probes as described by their size, partial nucleotide sequence and characteristic regulation pattern associated with neuroleptic administration.

Additionally, the present invention relates to vectors, host cells, antibodies, and recombinant methods for producing the polynucleotides and the polypeptides. One embodiment of the invention provides an isolated nucleic acid molecule comprising a polynucleotide chosen from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 which is regulated by neuroleptic administration. Also provided is an isolated nucleic acid molecule comprising a polynucleotide at least 95% identical to any one of these isolated nucleic acid molecules and an isolated nucleic acid molecule at least ten bases in length that is hybridizable to any one of these isolated nucleic acid molecules under stringent conditions. Any one of these isolated nucleic acid molecules can comprise sequential nucleotide deletions from either the 5′-terminus or the 3′-terminus. Further provided is a recombinant vector comprising any one of these isolated nucleic acid molecules and a recombinant host cell comprising any one of these isolated nucleic acid molecules. Also provided is the gene corresponding to the cDNA sequence of any one of these isolated nucleic acids.

Another embodiment of the invention provides an isolated polypeptide encoded by a polynucleotide chosen from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Also provided is an isolated nucleic acid molecule encoding any of these polypeptides, an isolated nucleic acid molecule encoding a fragment of any of these polypeptides, an isolated nucleic acid molecule encoding a polypeptide epitope of any of these polypeptides, and an isolated nucleic acid encoding a species homologue of any of these polypeptides. Preferably, any one of these polypeptides has biological activity. Optionally, any one of the isolated polypeptides comprises sequential amino acid deletions from either the C-terminus or the N-terminus. Further provided is a recombinant host cell that expresses any one of these isolated polypeptides.

Yet another embodiment of the invention comprises an isolated antibody that binds specifically to an isolated polypeptide encoded by a polynucleotide chosen from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID-NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. The isolated antibody, can be a monoclonal antibody or a polyclonal antibody.

Another embodiment of the invention provides a method for preventing, treating, modulating, or ameliorating a medical condition, such as a neuropsychiatric disorder, comprising administering to a mammalian subject a therapeutically effective amount of a polypeptide of the invention or a polynucleotide of the invention. In one preferred embodiment, a method for preventing, treating, moduclating or ameliorating schizophrenia is provided. In another preferred embodiment, a method for preventing, treating, modulating or ameliorating bipolar disorder is provided.

A further embodiment of the invention provides an isolated antibody that binds specifically to the isolated polypeptide of the invention. A preferred embodiment of the invention provides a method for preventing, treating, modulating, or ameliorating a medical condition, such as a neuropsychiatric disorder, comprising administering to a mammalian subject a therapeutically effective amount of the antibody. In one preferred embodiment, a method for preventing, treating, moduclating or ameliorating schizophrenia is provided. In another preferred embodiment, a method for preventing, treating, modulating or ameliorating bipolar disorders is provided.

An additional embodiment of the invention provides a method of diagnosing a pathological condition or a susceptibility to a pathological condition in a subject. The method comprises determining the presence or absence of a mutation in a polynucleotide of the invention. A pathological condition or a susceptibility to a pathological condition, such as a neuropsychiatric disorder, is diagnosed based on the presence or absence of the mutation. In one preferred embodiment, a method for diagnosing schizophrenia is provided. In another preferred embodiment, a method for diagnosing bipolar disorders is provided.

Even another embodiment of the invention provides a method of diagnosing a pathological condition or a susceptibility to a pathological condition, such as a neuropsychiatric disorder, in a subject. Especially preferred embodiments include methods of diagnosing schizophrenia and bipolar disorders. The method comprises detecting an alteration in expression of a polypeptide encoded by the polynucleotide of the invention, wherein the presence of an alteration in expression of the polypeptide is indicative of the pathological condition or susceptibility to the pathological condition. The alteration in expression can be an increase in the amount of expression or a decrease in the amount of expression. In a preferred embodiment a first biological sample is obtained from a patient suspected of having a neuropsychiatric disorder, for example, schizophrenia or a bipolar disorder, and a second sample from a suitable comparable control source is obtained. The amount of at least one polypeptide encoded by a polynucleotide of the invention is determined in the first and second sample. The amount of the polypeptide in the first and second samples is determined. A patient is diagnosed as having a neuropsychiatric disorder if the amount of the polypeptide in the first sample is greater than or less than the amount of the polypeptide in the second sample.

Another embodiment of the invention provides a method for identifying a binding partner to a polypeptide of the invention. A polypeptide of the invention is contacted with a binding partner and it is determined whether the binding partner effects an activity of the polypeptide.

Yet another embodiment of the invention is a method of identifying an activity of an expressed polypeptide in a biological assay. A polypeptide of the invention is expressed in a cell and isolated. The expressed polypeptide is tested for an activity in a biological assay and the activity of the expressed polypeptide is identified based on the test results.

Still another embodiment of the invention provides a substantially pure isolated DNA molecule suitable for use as a probe for genes regulated in neuropsychiatric disorders, chosen from the group consisting of the DNA molecules shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ED NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80.

Even another embodiment of the invention provides a kit for detecting the presence of a polypeptide of the invention in a mammalian tissue sample. The kit comprises a first antibody which immunoreacts with a mammalian protein encoded by a gene corresponding to the polynucleotide of the invention or with a polypeptide encoded by the polynucleotide in an amount sufficient for at least one assay and suitable packaging material. The kit can further comprise a second antibody that binds to the first antibody. The second antibody can be labeled with enzymes, radioisotopes, fluorescent compounds, colloidal metals, chemiluminescent compounds, phosphorescent compounds, or bioluminescent compounds.

Another embodiment of the invention provides a kit for detecting the presence of genes encoding a protein comprising a polynucleotide of the invention, or fragment thereof having at least 10 contiguous bases, in an amount sufficient for at least one assay, and suitable packaging material.

Yet another embodiment of the invention provides a method for detecting the presence of a nucleic acid encoding a protein in a mammalian tissue sample. A polynucleotide of the invention or fragment thereof having at least 10 contiguous bases is hybridized with the nucleic acid of the sample. The presence of the hybridization product is detected.

### BRIEF DESCRIPTION OF THE DRAWINGS

These and other features, aspects, and advantages of the present invention will become better understood with reference to the following description, appended claims, and accompanying drawings where:

FIG. 1 is a graphical representation of the results of TOGA™ runs using a 5′ PCR primer with parsing bases CTAA (SEQ ID NO:66) and the universal 3′ PCR primer (SEQ ID NO:23) showing PCR products produced from mRNA extracted from the striatum/nucleus accumbens of mice treated with 7.5 mg/kg of clozapine for the following durations: control (no clozapine) (Panel A), 45 minutes (Panel B), 7 hours (Panel C), 5 days (Panel D), 12 days (Panel E), and 14 days (Panel F), where the vertical index line indicates a PCR product of about 461 b.p. that is expressed to a greater level in the 12 day clozapine-treated sample than in the other samples. The horizontal axis represents the number of base pairs of the molecules in these samples and the vertical axis represents the fluorescence measurement in the TOGA™ analysis (which corresponds to the relative expression of the molecule of that address). The results of the TOGA™ runs have been normalized using the methods described in pending U.S. patent application Ser. No. 09/318,699/U.S., and pending PCT Application Serial No. PCT/US00/14159, both entitled Methods and System for Amplitude Normalization and Selection of Data Peaks (Dennis Grace, Jayson Durham); and pending U.S. patent application Ser. No. 09/318,679/U.S. and pending PCT Application Serial No. PCT/US00/14123, both entitled Methods for Normalization of Experimental Data (Dennis Grace, Jayson Durham) all of which are incorporated herein by reference. The vertical line drawn through the five panels represents the DST molecule identified as CLZ_{—}43 (SEQ ID NO:37).

FIG. 2 presents a graphical example of the results obtained when a DST is verified by the Extended TOGA™ method using a primer generated from a cloned product (as described below). The PCR product corresponding to SEQ ID NO:37 (CLZ_{—}43) was cloned and a 5′ PCR primer was built from the cloned DST (SEQ ID NO:94). The product obtained from PCR with this primer (SEQ ID NO:72) and the universal 3′ PCR primer (SEQ ID NO:23) (as shown in the top panel) was compared to the length of the original PCR product that was produced in the TOGA™ reaction with mRNA extracted from the striatum/nucleus accumbens of mice treated with 7.5 mg/kg of clozapine for 12 days using a 5′ PCR primer with parsing bases CTAA (SEQ ID NO:66) and the universal 3′ PCR primer (SEQ ID NO:23) (as shown in the middle panel). Again, for all panels, the number of base pairs is shown on the horizontal axis, and fluorescence intensity (which corresponds to relative expression) is found on the vertical axis. In the bottom panel, the traces from the top and middle panels are overlaid, demonstrating that the peak found using an extended primer from the cloned DST is the same number of base pairs as the original PCR product obtained through TOGA™ as CLZ_{—}43 (SEQ ID NO:37). The bottom panel thus illustrates that CLZ_{—}43 (SEQ ID NO:37) was the DST amplified in Extended TOGA™.

FIG. 3A-D compares the results from Real Time PCR validation (A) (as described below) to the TOGA™ result from three different experiments: the original clozapine experiment (B), a repeated clozapine experiment performed in duplicate (C), and a haloperidol experiment in duplicate (D). As the tables and graphs illustrate, TOGA™ and Real Time PCR'show that the DST CLZ_{—}43 (SEQ ID NO:37) increases in expression in clozapine treated mice, while is not responsive to haloperidol treatment. Table 5 lists the 5′ and 3′ primers used in these Real-Time PCR studies.

FIG. 4A-F is an in situ hybridization analysis using an antisense cRNA probe directed against the 3′end of CLZ_{—}43 (SEQ ID NO:37) in saline, clozapine, or haloperidol treated mice. FIG. 4A-F demonstrates the pattern of CLZ_{—}43 mRNA expression in coronal sections where A, B and C were sectioned at the level of the striatum (containing nucleus accumbens, Nacc, caudateputamen, Cpu, and neocortex, NC) and D, E, and F were sectioned at the level of the thalamus (Thal), hippocampus (Hipp), and hypothalamus (Hyp). A low level of expression was observed in the striatum, and treatment with either haloperidol or clozapine resulted in increased expression in the neocortex and in the striatum in mouse brain (B and C). Comparison with brain sections obtained from control mice showed that CLZ_{—}43 expression is increased approximately 3-fold by chronic treatment with clozapine or haloperidol.

FIG. 5A-D compares the results from Real Time PCR validation (A) (as described below) to the TOGA™ result from three different experiments: the original clozapine experiment (B), a repeated clozapine experiment performed in duplicate (C), and a haloperidol experiment in duplicate (D). As the tables and graphs illustrate, the Real Time PCR shows that the mouse sequence homolog to human KIAA1451 (SEQ ID NO:101) increases in expression in both clozapine (2.09-fold) and haloperidol (2.57-fold) treated mice. Due to the different Real Time PCR profile compared to TOGA™ profile for the haloperidol response, it is believed that the mouse KIAA-related sequence represents a neuroleptic responsive gene that is related, but distinct from the DST CLZ_{—}43 (SEQ ID NO:37). Table 5 lists the 5′ and 3′ primers used in these Real-Time PCR studies.

FIG. 6 is a graphical representation of the results of TOGA™ analysis, similar to FIG. 1, using a 5′ PCR primer with parsing bases TTGT (SEQ ID NO: 26) and the universal 3′ primer (SEQ ID NO: 23), showing PCR products produced from mRNA extracted from the striatum/nucleus accumbens of mice treated with 7.5 mg/kg clozapine as follows: control (no clozapine) (Panel A), 45 minutes (Panel B), 7 hours (Panel C), 5 days (Panel D), 12 days (Panel E), and 14 days (Panel F), where the vertical index line indicates a PCR product of about 266 b.p. that is present in the control sample, is-down-regulated within 45 minutes in the clozapine-treated sample, and remains down-regulated for 14 days in the presence of clozapine. The vertical line drawn through the five panels represents the DST molecule identified as CLZ_{—}40 (SEQ ID NO:12).

FIG. 7 is a graphical representation of the results of TOGA™ analysis using a 5′ PCR primer with parsing bases TTGT (SEQ ID NO: 26) and the universal 3′ primer (SEQ ID NO: 23), showing PCR products produced from mRNA extracted from the brain of morphine-treated mice as follows: control striatum (PS) (Panel A), acutely treated striatum (AS) (Panel B), withdrawal striatum (WS) (Panel C), control amygdala (PA) (Panel D), acutely treated amygdala (AA) (Panel E), chronically treated amygdala (TA) (Panel F), and withdrawal amygdala (WA) (Panel G), where the vertical index line indicates a PCR product of about 266 b.p. that is more abundant in control striatum than control amygdala and is differentially regulated by morphine in striatum versus amygdala.

FIG. 8 shows a Northern Blot analysis of DST CLZ_{—}40 (TTGT 266) (SEQ ID NO: 12), where an agarose gel containing poly A enriched mRNA from the striatum/nucleus accumbens of clozapine-treated mice as well as size standards was blotted after electrophoresis and probed with radiolabelled CLZ_{—}40. Mice were treated with clozapine (7.5 mg/kg) for the following time durations before mRNA extraction: control (no clozapine), 45 minutes, 7 hours, 5 days, 12 days, and 14 days.

FIG. 9 is a graphical representation comparing the results of the TOGA™ analysis of clone CLZ_{—}40 (SEQ ID NO: 12) shown in FIG. 6 and the Northern Blot analysis of clone CLZ_{—}40 shown in FIG. 8.

FIG. 10A-D compares the results from Real Time PCR validation (A) (as described below) to the TOGA™ result from three different experiments: the original clozapine experiment (B), a repeated clozapine experiment performed in duplicate (C), and a haloperidol experiment in duplicate (D). As the tables and graphs illustrate, the TOGA™ and Real Time PCR show that the DST CLZ_{—}40 (SEQ ID NO:12) decreases in expression in response to both clozapine and haloperidol treatment. Table 5 lists the 5′ and 3′ primers used in these Real-Time PCR studies.

FIG. 11A-B is an in situ hybridization analysis, showing DST CLZ_{—}40 (SEQ ID NO: 12) mRNA expression in mouse brain using an antisense cRNA probe directed against the 3′ end of CLZ_{—}40, where 15A shows expression in the nucleus accumbens (Acb) and pyriform cortex (Pir) and 15B shows expression in the dentate gyrus (DG).

FIG. 12 is a graphical representation of the results of TOGA™ analysis using a 5′ PCR primer with parsing bases CACC (SEQ ID NO: 25) and universal 3′ primer (SEQ ID NO: 23), showing PCR products produced from mRNA extracted from the striatum/nucleus accumbens of mice treated with 7.5 mg/kg of clozapine for the following durations: control (no clozapine) (Panel A), 45-minutes (Panel B), 7 hours (Panel C), 5 days (Panel D), 12 days (Panel E), and 14 days (Panel F), where the vertical index line indicates a PCR product of about 201 b.p. that is present in the control sample and increasingly enriched over time in the clozapine-treated samples. The vertical line drawn through the five panels represents the DST molecule identified as CLZ_{—}5 (SEQ ID NO:2).

FIG. 13 shows a Northern Blot analysis of clone CLZ_{—}5 (CACC 201) (SEQ ID NO: 2), where an agarose gel containing poly A enriched mRNA from the striatum/nucleus accumbens of mice treated with clozapine as well as size standards was blotted after electrophoresis and probed with radiolabelled CLZ_{—}5. Mice were treated with clozapine (7.5 mg/kg) for the following time durations before mRNA extraction: control (no clozapine), 45 minutes, 7 hours, 5 days, 12 days, and 14 days.

FIG. 14 is a graphical representation comparing the results of the TOGA analysis of DST CLZ 5 (SEQ ID NO: 2) shown in FIG. 12 and the Northern Blot analysis of clone CLZ_{—}5 shown in FIG. 13.

FIG. 15A-D compares the results from Real Time PCR validation (A) (as described below) to the TOGA™ result from three different experiments: the original clozapine experiment (B), a repeated clozapine experiment performed in duplicate (C), and a haloperidol experiment in duplicate (D). As the tables and graphs illustrate, the TOGA™ and Real Time PCR show that the DST CLZ_{—}5 (SEQ ID NO:2) increases in expression in response to both clozapine and haloperidol treatment. Table 5 lists the 5′ and 3′ primers used in these Real-Time PCR studies.

FIG. 16A-C is an in situ hybridization analysis using an antisense cRNA probe directed against the 3′ end of CLZ_{—}5 (SEQ ID NO:2), showing the pattern of CLZ_{—}5 mRNA expression in mouse anterior brain (16A), midbrain (16B), and posterior brain (16C), where CLZ_{—}5 is expressed in scattered glial cells and white matter tracts.

FIG. 17A-I is an in situ hybridization analyses, using an antisense cRNA probe directed against the 3′ end of CLZ_{—}5 (SEQ ID NO:2), showing CLZ_{—}5 mRNA expression in mouse anterior brain (17A-C), midbrain (17D-F), and posterior brain (17G-I) in saline-treated mice (top row), mice treated with clozapine for 5 days (middle row), and mice treated with clozapine for 14 days (bottom row), where the clozapine treatment induces expression in the glial cells.

FIG. 18A-H shows a darkfield photomicrograph of various brain regions, including the corpus callosum (cc, FIG. 189A, E); caudate putamen (CPu, FIG. 18B, F); anterior commissure (aca, FIG. 18C, G); and globus pallidus (GP, FIG. 18D, H) in control (18A-D) and clozapine-treated (18E-H) animals. The figure demonstrates upregulated ApoD mRNA expression in various brain regions.

FIG. 19A-D shows a darkfield photomicrograph in the internal capsule (ic) (19A, B) and a brightfield view of the optic tract (opt) (19C, D) from control (19A, C) and clozapine-treated (19B, D) animals. The figure demonstrates up-regulated apoD mRNA expression in the internal capsule (ic).

FIG. 20A-F shows GFAP and apoD co-localization in the striatum (20A, B, D, E) and optic tract (20C, F) of control saline (20A, B, C) and clozapine-treated animals (20D, E, F), with thick arrows designating the co-localization of GFAP and apoD mRNA and thin arrows designating the expression of apoD only; 20G-H shows apoD immunohistochemistry with an anti-human apoD primary antibody (Novocastra, Newcastle, UK) in the optic tract of control saline (20G) and clozapine-treated animals (20H).

FIG. 21 shows a Northern Blot analysis of clone CLZ_{—}5 (SEQ ID NO: 2), where an agarose gel containing poly A enriched mRNA from cultured glial cells treated with clozapine as well as size standards was blotted after electrophoresis and probed with radiolabelled CLZ_{—}5. Cultured glial cells were treated with different concentrations of clozapine for different lengths of time before mRNA extraction as follows: Lane A=control (no clozapine), Lane B=100 nM clozapine, 1 day, Lane C=1 μM clozapine, 1 day, Lane D=100 nM clozapine, 1 week, Lane E=1 μM clozapine, 1 week.

FIG. 22A-B are Western blot analyses showing the distribution of apoD expression in human brain, wherein Western blots containing 50 μg total protein/lane were probed with a monoclonal antibody directed against human apoD and enhanced chemiluminescence (ECL) was used to detect immunoreactivity. FIG. 22A was visualized by 30 second exposure to autoradiography film and FIG. 22B was visualized by 90 second exposure to autoradiography film. Caudate=Caud; Putamen=Put; Dentate gyrus=Dent; Subiculum=Subic; Substantia nigra=SN; parahippocampal gyrus=PHG.

FIG. 23A and B shows Western blot analyses (23A) and densitrometric data (23B) demonstrating apoD expression in dorsolateral prefrontal cortex of eight control (Con-1 to Con-8) and eight schizophrenic subjects (Sch-1 to Sch-8), wherein 50 μg total protein/lane were probed with a monoclonal antibody directed against human ApoD and enhanced chemiluminescence (ECL) was used to detect immunoreactivity.

FIG. 24A-I is a graphical representation of ELISA assays which show ApoD levels in dorsolateral prefrontal cortex (BA9; A, G), occipital cortex (BA18; B, H) and caudate (caud; C, I), substantia nigra (SN; D), cerebellum (Cb; E) and hippocampus (Hipp; F) of control versus schizophrenic subjects (24A-F) and ApoD levels in dorsolateral prefrontal cortex (BA9), occipital cortex (BA18) and caudate of control versus bipolar subjects (24G-I). Significant differences are indicated by asterisks as determined by student's t test (two-tailed) where *** indicates P=0.0002, ** indicates P=0.02, and * indicates P=0.04.

FIG. 25 is a graphical representation of ELISA assays showing ApoD levels in the serum of control subjects and schizophrenic subjects using purified apoD as a standard, wherein asterisks denote a significant decrease, P=0.0083.

FIG. 26A-B are scatter plots showing ApoD levels in the serum of male versus female subjects (26A) and subjects ranging in age from 20-65 (26B).

FIG. 27 shows regional ApoD gene expression in the brain was compared in young nontransgenic (Yg-NT), young transgenic (Yg-TG), aged nontransgenic (Aged-N) and aged transgenic (Aged-TG) mice. At the gross level, apoD expression in young PDAPP Tg mice did not differ significantly from young wild type mice. In both aged wild type and PDAPP mice apoD increased significantly as compared to young mice. These increases in expression were most notable in the white matter tracts: hippocampal fimbria, corpus callosum, septal white matter tracts. Comparison between aged wild type and PDAPP mice revealed that the PDAPP mice had greater apoD expression as compared to the wild type. Representative sections were taken at four different levels. Panel A taken at the level of the caudatoputamen (CP) demonstrating gene expression in the corpus callosum (cc) and septal white matter tracts (sp). Panel B taken at the level of the globus pallidus (GP) demonstrating gene expression in the hippocampal fimbria (fi) and corpus callosum (cc). Panel C, D at the level of the hippocampus (Hipp) and thalamus (Th) demonstrating gene expression in the corpus callosum (cc).

FIG. 28 shows at the cellular level, the number of individual glial cells that express apoD in the corpus callosum (A) and hippocampal fimbria (B) of the PDAPP mice as compared to the wild type mice was significant. In the medial corpus callosum dorsal to the hippocampus apoD gene expression is increased moderately in the young transgenic mice (Y-Tg) as compared to the young nontransgenic mice (Y-NT). ApoD mRNA expression in the medial corpus callosum is significantly increased in the aged transgenic (Aged-Tg) as compared to the aged nontransgenic (Aged-NT). In the hippocampal fimbria (Hipp-fi) apoD gene expression is increased significantly in the Aged Tg as compared to Aged-NT.

FIG. 29 shows the increase in ApoD expression at the cellular level in the corpus callosum and hippocampal fimbria were quantified by determining the number of cells expressing apoD mRNA within a defined field of view. Cell counts were performed using a 20× objective in both brightfield and darkfield on 4 different slices from each of the 2 regions, the corpus callosum (A) and the hippocampal fimbria (B). A total of 4 animals from each group was analyzed (young nontransgenic, Yg-NT; young transgenic, Yg-Tg; aged transgenic, Aged-NT; aged nontransgenic, Aged-Tg). An approximate 300% increase in apo-D positive cells was observed in both the corpus callosum (A) and hippocampal fimbria (B) of the Aged-Tg vs. Aged-NT. A 447% increase in number of apoD positive cells in the corpus callosum (A) and 613% increase in the hippocampal fimbria (B3) in the Aged-Tg vs. Yg-Tg was observed. *, p<0.05; ***p<0.0001 as determined by one way analysis of variance with a Bonferroni post-test.

FIG. 30A-B is an in situ hybridization analysis using an antisense cRNA probe directed against the 3′ end of DST CLZ_{—}3 (SEQ ID NO: 1), showing the pattern of CLZ_{—}3 mRNA expression in a coronal section through the hemispheres at level of hippocampus (30A) and cross section through midbrain (30B) in mouse brain.

FIG. 31A-B is an in situ hybridization analysis using an antisense cRNA probe directed against the 3′ end of CLZ_{—}16 (SEQ ID NO:15), showing the pattern of CLZ_{—}16 mRNA expression in coronal sections through hemispheres in mouse brain. FIG. 31A shows dense labeling in the cortex and surrounding the hippocampal formation as well as moderate labeling in the dorsal thalamus and posterior brain. FIG. 31B shows uniform labeling throughout.

FIG. 32 shows CLZ_{—}17 (SEQ ID NO: 28) mRNA expression in the brain was determined by in situ hybridization using riboprobes specific to the DST. In (A) CLZ_{—}17 expression was observed in the septal nucleus (SPT). In (B) CLZ_{—}17 expression was observed in the hypothalamic nuclei (HYP) and SPT. In (C) CLZ_{—}17 was observed in the hippocampus (HIP) and the HYP. In (D) CLZ 17 was observed in the amygdala (AMYG), the HYP, and the HIP.

FIG. 33A-B is an in situ hybridization analysis using an antisense cRNA probe directed against the 3′ end of CLZ_{—}24 (SEQ ID NO:7), showing the pattern of CLZ_{—}24 mRNA expression in a coronal section through the hemispheres (33A) and cross section through the brainstem (33B) in mouse brain.

FIG. 34A-B is an in situ hybridization analysis using an antisense cRNA probe directed against the 3′ end of CLZ_{—}26 (SEQ ID NO:29), showing the pattern of CLZ_{—}26 mRNA expression in a coronal section of the hemispheres at the level of hippocampal formation (34A) and coronal section of the hemispheres at the level of striatum (34B) in mouse brain.

FIG. 35A-B is an in situ hybridization analysis using an antisense cRNA probe directed against the 3′ end of CLZ_{—}28 (SEQ ID NO:30), showing the pattern of CLZ_{—}28 mRNA expression in a coronal section through the hemispheres at the level of hippocampus (35A) and coronal section through the posterior region of hemispheres (35B) in mouse brain.

FIG. 36A-B is an in situ hybridization analysis using an antisense cRNA probe directed against the 3′ end of CLZ 34 (SEQ ID NO:9) showing the pattern of CLZ_{—}34 mRNA expression in a coronal section through the hemispheres at the level of hippocampus (36A) and cross section through the midbrain (36B) in mouse brain.

FIG. 37 is a graphical representation of a Northern Blot analysis of clone CLZ_{—}38 (TGCA 109) (SEQ ID NO:11), where an agarose gel containing poly A enriched mRNA from the striatum/nucleus accumbens of clozapine-treated mice as well as size standards was blotted after electrophoresis and probed with radiolabelled CLZ_{—}38. Mice were treated with clozapine (7.5 mg/kg) for the following time durations before mRNA extraction: control (no clozapine), 45 minutes, 7 hours, 5 days, 12 days, and 14 days.

FIG. 38 shows CLZ_{—}38 mRNA expression in the brain was determined by in situ hybridization using riboprobes specific to the DST. CLZ_{—}38 expression was observed primarily in the white matter tracts of the brain. In (A,B) CLZ_{—}38 is expression is observed in the corpus callosum (cc) and anterior commisure (ac). In (B) CLZ_{—}38 is also expression is observed in the white matter of the septum (sp). In (C) CLZ_{—}38 is expressed by cells in the hippocampal fimbria (fi). In (D) CLZ_{—}38 expression is observed in the cc, fi, and optic tract (opt).

FIG. 39 is a graphical representation of a Northern Blot analysis of clone CLZ_{—}44 (ACGG 352) (SEQ ID NO:38), where an agarose gel containing poly A enriched mRNA from the striatum/nucleus accumbens of clozapine-treated mice as well as size standards was blotted after electrophoresis and probed with radiolabelled CLZ_{—}44. Mice were treated with clozapine (7.5 mg/kg), haloperidol (4 mg/kg), or ketanserin (4 mg/kg) for two weeks before mRNA extraction.

FIG. 40A-B is an in situ hybridization analysis using an antisense cRNA probe directed against the 3′ end of CLZ_{—}44 (SEQ ID NO:38), showing the pattern of CLZ_{—}44 mRNA expression in a coronal section showing labeling in the hippocampus, hypothalamus, and temporal cortex (40A) and coronal section showing cortical labeling (40B) in mouse brain.

FIG. 41A-B is an in situ hybridization analysis using an antisense cRNA probe directed against the 3′ end of CLZ_{—}64 (SEQ ID NO:48), showing the pattern of CLZ_{—}64 mRNA expression in different coronal sections of the hemispheres in mouse brain.

### DETAILED DESCRIPTION OF THE PREFERRED EMBODIMENTS

The present invention and the methods of obtaining and using the present invention will be described in detail after setting forth some preliminary definitions.

Definitions

The following definitions are provided to facilitate understanding of certain terms used in the present invention. Many of the techniques described herein are described in Dracopoli et al., Current Protocols in Human Genetics, John Wiley and Sons, New York (1999), and Ausubel et al., Current Protocols in Molecular Biology, John Wiley and Sons, New York (2000), both of which are incorporated herein by reference.

An “isolated nucleic acid” refers to a nucleic acid the structure of which is not identical to that of any naturally occurring nucleic acid or to that of any fragment of naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; (d) a recombinant nucleotide sequence that is part of a hybrid gene, i.e., a gene encoding a fusion protein; and (e) a nucleic acid synthesized through chemical means.

An “isolated polypeptide” refers to a polypeptide removed from its original environment (e.g., the natural environment if it is naturally occurring), and thus is altered “by the hand of man” from its natural state.

An “isolated antibody” refers to an antibody removed from its original environment (e.g., the natural environment if it is naturally occurring), and thus is altered “by the hand of man” from its natural state.

“Isolated” refers to material removed from its original environment (e.g., the natural environment if it is naturally occurring), and thus is altered “by the hand of man” from its natural state.

“Polynucleotide” or “polynucleotide of the invention” or “polynucleotide of the present invention” refers to a molecule having a nucleic acid sequence contained in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. For example, the polynucleotide can contain all or part of the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. A polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms. A “polynucleotide” of the present invention also includes those polynucleotides capable of hybridizing, under stringent hybridization conditions, to sequences contained in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 or the complement thereof, or the cDNA. “Stringent hybridization conditions” refers to an overnight incubation at 42° C in a solution comprising 50% formamide, 5X SSC (5×SSC =750 mM NaCl, 75 mM sodium citrate, 50 mM sodium phosphate pH 7.6), 5× Denhardt's solution, 10% dextran sulfate, and 20 μg/ml denatured, sheared salmon sperm DNA, followed by washing the filters in 0.1×SSC at about 65° C. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 ug/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Note that variations in the above conditions may be accomplished through the inclusion and/or substitution of alternate blocking reagents used to suppress background in hybridization experiments. Typical blocking reagents include Denhardt's reagent, BLOTTO (5% w/v non-fat dried milk in phosphate buffered saline (“PBS), heparin, denatured salmon sperm DNA, and other commercially available proprietary formulations. The inclusion of specific blocking reagents may require modification of the hybridization conditions described above, due to problems with compatibility. Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

“Polypeptide” or “polypeptide of the invention” or “polypeptide of the present invention” refers to a molecule having a translated amino acid sequence generated from the polynucleotide as broadly defined. The translated amino acid sequence, beginning with the methionine, is identified although other reading frames can also be easily translated using known molecular biology techniques. The polypeptides produced by the translation of these alternative open reading frames are specifically contemplated by the present invention. The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. See references below. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, e.g., T. E. Creighton, Ed., Proteins—Structure And Molecular Properties, 2nd Ed., W. H. Freeman and Company, New York (1993); B. C. Johnson, Ed., Posttranslational Covalent Modification Of Proteins, Academic Press, New York, pgs. 1-12 (1983); Seifter et al., Meth. Enzymol., 182:626-646 (1990); Rattan et al., Ann. N.Y. Acad. Sci., 663:48-62 (1992)).

A polypeptide has “biological activity” when the polypeptide has structural, regulatory or biochemical functions of a naturally occurring molecule. Biological activity can be measured by several kinds of biological assays, both in vitro (e.g., cell cultures) or in vivo (e.g., behavioral or metabolic assays). In these cases, the potency of the biological activity is measured by its dose-response characteristics; in the case of polypeptides with activity similar to the polypeptide of the present invention, the dose-response dependency will be substantially similar in a given activity as compared to the polypeptide of the present invention. Polypeptides may derive their “biological activity” through binding to specific cellular receptors, which-mediate secondary signals to the target cell or tissue. In other cases, they may have direct effects in the absence of receptor mediated binding or signaling. For example, peptides may interact directly with other proteins or other molecules, and alter their conformation of function, or they may block the binding of a third molecule to the same interaction site, thereby affecting the singal normally mediated between the two molecules.

“DNA” refers to deoxyribonucleic acid.

“RNA” refers to ribonucleic acid.

“mRNA” refers to messenger ribonucleic acid.

“cDNA” refers to a deoxyribonucleic acid that is complementary to an mRNA.

“Gene” refers to a region of DNA that controls a discrete hereditary characteristic, usually corresponding to a single protein or RNA. This definition includes the entire functional unit encompassing coding DNA sequence, the regions preceding and following the coding region (leader or trailer), noncoding regulatory DNA sequences, and introns.

“Codon” refers to the three-nucleotide sequence of an mRNA molecule that codes for one specific amino acid.

“Vector” refers to a vehicle for transfer of DNA into a recipient cell.

“Silent mutation” or “silent substitution” refers to a mutation that causes no functional change in the gene product.

“Phenotype” refers to the appearance, behavior, or other characteristics of a cell or individual due to actual expression, or pattern of expression, of a specific gene or set of genes. Differences in phenotype may be due to changes in the expression or pattern of expression of a specific gene or set of genes, or to differences in the biological activity of one or more genes. These differences may be a result of polymorphic or allelic differences in the coding region of the specific genes or in their regulatory sequences, or to other genetic variations (e.g., new mutations).

“Hybridization” refers to the time- and temperature-dependent process by which two complementary single-stranded polynucleotides associate to form a double helix.

“Probe” refers to a polynucleotide, often radiolabelled, used to detect complementary sequences, e.g. an mRNA used to locate its gene by a corresponding nucleic acid blotting method.

“Conservative amino acid substitution” refers to a substitution between similar amino acids that preserves an essential chemical characteristic of the original polypeptide.

“Phage” refers to a virus that infects bacteria. Many phage have proved useful in the study of molecular biology and as vectors for the transfer of genetic information between cells.

“Plasmid” refers to a self-replicating extra-chromosomal element, usually a small segment of duplex DNA that occurs in some bacteria; used as a vector for the introduction of new genes into bacteria.

“Retrovirus” refers to a virus with an RNA genome that may be either an mRNA, (+)-RNA, or its complement, (−)-RNA. Class 1 contains (+)-RNA; class 2, (−)-RNA, which is the template for an RNA-dependent RNA polymerase; class 3, double-stranded RNA, in which (+)-RNA is synthesized by an RNA-dependent RNA polymerase; class 4, retrovirus, in which (+)-RNA is a template for an RNA-dependent DNA polymerase (a reverse transcriptase). A Retrovirus may be used as a vector for the introduction of genes into mammalian cells. “Triple Helix” refers to the tertiary structure of collagen that twist

s three polypeptide chains around themselves; also a triple-stranded DNA structure that involves Hoogstein base pairing between B-DNA and a third DNA strand that occupies the major groove.

“Antibody” refers to an immunoglobulin molecule that reacts specifically with another (usually foreign) molecule, the antigen.

“Monoclonal antibody (mAb)” refers to an immunoglobulin preparation that is completely homogeneous, due to its formation by daughters of a single progenitor cell that has been programmed for the synthesis and secretion of one specific antibody.

“Polyclonal antibody” refers to a heterogeneous immunoglobulin preparation that contains antibodies directed against one or more determinants on an antigen; the product of daughters of several progenitor cells that have been programmed for immunoglobulin synthesis and secretion.

“Complementary” as used in nucleic acid chemistry, is descriptive of the relationship between two polynucleotides that can combine in an antiparallel double helix; the bases of each polynucleotide are in a hydrogen-bonded inter-strand pair with a complementary base, A to T (or U) and C to G. In protein chemistry, the matching of shape and/or charge of a protein to a ligand.

“C-terminus” refers to, in a polypeptide, the end with a free carboxyl group.

“N-terminus” refers to, in a polypeptide, the end with a free amino group.

A “secreted” protein refers to those proteins capable of being directed to the endoplasmic reticulum, secretory vesicles, or the extracellular space as a result of a signal sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Variant” refers to a polynucleotide or polypeptide differing from the polynucleotide or polypeptide of the present invention, but retaining essential properties thereof. In general, variants have close similarity overall and are identical in many regions to the polynucleotide or polypeptide of the present invention.

“Identity” per se has an art-recognized meaning and can be calculated using published techniques. (See, e.g., Lesk, Ed., Computational Molecular Biology, Oxford University Press, New York, (1988); Smith, Ed., Biocomputing: Informatics And Genome Projects, Academic Press, New York, (1993); Griffin and Griffin, Eds., Computer Analysis Of Sequence Data, Part I, Humana Press, New Jersey, (1994); von Heinje, Sequence Analysis In Molecular Biology, Academic Press, (1987); and Gribskov and Devereux, Eds., Sequence Analysis Primer, M Stockton Press, New York, (1991)). While there exists a number of methods to measure identity between two polynucleotide or polypeptide sequences, the term “identity” is well known to skilled artisans (Carillo et al., SIAM J Applied Math., 48:1073 (1988)). Methods commonly employed to determine identity or similarity between two sequences include, but are not limited to, those disclosed in “Guide to Huge Computers,” Martin J. Bishop, Ed., Academic Press, San Diego, (1994) and Carillo et al., (1988), Supra.

“Epitopes” refer to polypeptide fragments having antigenic or immunogenic activity in an animal, especially in a human. A preferred embodiment of the present invention relates to a polypeptide fragment comprising an epitope, as well as the polynucleotide encoding this fragment. A region of a-protein molecule to which an antibody can bind is defined as an “antigenic epitope.” In contrast, an “immunogenic epitope” is defined as a part of a protein that elicits an antibody response. (See, e.g., Geysen et al., Proc. Natl. Acad. Sci. USA, 81:3998-4002 (1983)).

“Homologous” means corresponding in structure, position, origin or function.

A “homologous polynucleotide” refers to a polynucleotide which encodes a homologous polypeptide.

A “homologous nucleic acid molecule” refers to a nucleic acid molecule which encodes a homologous polypeptide.

A “homologous polypeptide” refers to a polypeptide having any of the following characteristics with respect to the polypeptides of the present invention: similar function, similar amino acid sequence, similar subunit structure and formation of a functional heteropolymer, immunological cross-reaction, similar expression profile, similar subcellular location, similar substrate specificity, or similar response to specific inhibitors.

“ELISA” refers to an enzyme-linked immunosorbent assay that employs an antibody or antigen bound to a solid phase and an enzyme-antigen or enzyme-antibody conjugate to detect and quantify the amount of an antigen present in a sample.

A “specific binding agent” refers to a molecular entity capable of selectively binding a reagent species of the present invention or a complex containing such a species, but is not itself a polypeptide or antibody molecule composition of the present invention.

The word “complex” as used herein refers to the product of a specific binding reaction such as an antibody-antigen or receptor-ligand reaction. Exemplary complexes are immunoreaction products.

As used herein, the terms “label” and “indicating means” in their various grammatical forms refer to single atoms and molecules that are either directly or indirectly involved in the production of a detectable signal to indicate the presence of a complex.

The term “package” refers to a solid matrix or material such-as glass, plastic (e.g., polyethylene, polypropylene, or polycarbonate), paper, foil and the like capable of holding within fixed limits a polypeptide, polyclonal antibody, or monoclonal antibody of the present invention. Thus, for example, a package can be a glass vial used to contain milligram quantities of a contemplated polypeptide or antibody or it can be a microtiter plate well to which microgram quantities of a contemplated polypeptide or antibody have been operatively affixed (i.e., linked) so as to be capable of being immunologically bound by an antibody or antigen, respectively.

“Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

“DST” refers to a Digital Sequence Tag, i.e., a polynucleotide that is an expressed sequence tag of the 3′ end of an mRNA.

Other terms used in the fields of biotechnology and molecular and cell biology as used herein will be as generally understood by one of ordinary skill in the applicable arts.

### Detailed Description of the Invention

Background

The following experiments were conducted to identify gene expression associated with the use of different neuroleptic agents. These experiments are intended to illustrate the invention, and are not to be construed as limiting the scope of the invention.

### EXAMPLE 1

### Identification and Characterization of Polynucleotides Regulated by Neuroleptic Drugs

Male C57B1/6J mice (20-28 g) were housed in groups of four on a standard 12/12 hour light-dark cycle with ad libitum access to standard laboratory chow and tap water. For the experimental paradigms, mice were divided into groups of 25 and subjected to the following treatments:

Control groups: Mice received a single injection of sterile saline (0.1 ml volume), or no injection, and were sacrificed after 45 minutes.

Acute neuroleptic treatment: Mice received a single intraperitoneal injection of the atypical neuroleptic clozapine (7.5 mg/kg). Animals were sacrificed after 45 minutes.

Chronic neuroleptic treatment: Mice received daily subcutaneous injections of clozapine (7.5 mg/kg) or haloperidol (4 mg/kg) for time periods of 5 days to 2 weeks.

All animals were sacrificed in their cages with CO₂ at the indicated times. Brains were rapidly removed and placed on ice. The striatum, including the nucleus accumbens, were dissected out and placed in ice-cold phosphate-buffered saline. The cytoplasmic RNA was isolated by phenol:chloroform extraction of the homogenized tissue according to the method described in Schibler et al., J. Mol. Bio., 142, 93-116 (1980). Poly A enriched mRNA was prepared from cytoplasmnic RNA using well-known methods of oligo dT chromatography. Isolated RNA was then analyzed using a method of simultaneous sequence-specific identification of mRNAs known as TOGA™ (TOtal Gene expression Analysis) described below.

The TOGA™ Process

Isolated RNA was analyzed using a method of simultaneous sequence-specific identification of mRNAs known as TOGA™ (TOtal Gene expression Analysis) described in Sutcliffe, et al. Proc. Natl. Acad. Sci. USA, 97(5):1976-1981 (2000); International published application WO 00/26406; U.S. Pat. No. 5,459,037; U.S. Pat. No. 5,807,680; U.S. Pat. No. 6,030,784; U.S. Pat. No. 6,096,503 and U.S. Pat. 6,110,680, hereby incorporated herein by reference. Preferably, prior to the application of the TOGA™ technique, the isolated RNA was enriched to form a starting polyA-containing mRNA population by methods known in the art. In a preferred embodiment, the TOGA™ method further comprised an additional PCR step performed using four 5′ PCR primers in four separate reactions and cDNA templates prepared from a population of antisense cRNAs. A final PCR step that used 256 5′ PCR primers in separate reactions produced PCR products that were cDNA fragments that corresponded to the 3′-region of the starting mRNA population. The produced PCR products were then identified by: a) the initial 5′ sequence comprising the sequence remainder of the recognition site of the restriction endonuclease used to cut and isolate the 3′ region plus the sequence of the preferably four parsing bases immediately 3′ to the remainder of the recognition site, preferably the sequence of the entire fragment, and b) the length of the fragment. These two parameters, sequence and fragment length, were used to compare the obtained PCR products to a database of known polynucleotide sequences. Since the length of the obtained PCR products includes known vector sequences at the 5′ and 3′ ends of the insert, the sequence of the insert provided in the sequence listing is shorter than the fragment length that forms part of the digital address.

The method yields Digital Sequence Tags (DSTs), that is, polynucleotides that are expressed sequence tags of the 3′ end of mRNAs. DSTs that showed changes in relative levels during intraperitoneal injection clozapine were selected for further study. The intensities of the laser-induced fluorescence of the labeled PCR products were compared across samples isolated.

In general, double-stranded cDNA is generated from poly(A)-enriched cytoplasmic RNA extracted from the tissue samples of interest using an equimolar mixture or set of all 48 5′-biotinylated anchor primers to initiate reverse transcription. One such suitable set is G-A-A-T-T-C-A-A-C-T-G-G-A-A-G-C-G-G-C-C-G-C-A-G-G-A-A-T-T-T-T-T-T-T-T-T-T-T-T-T-T-T-T-T-T-V-N-N (SEQ ID NO: 20), where V is A, C or G and N is A, C, G or T. One member of this mixture of 48 anchor primers initiates synthesis at a fixed position at the 3′ end of all copies of each mRNA species in the sample, thereby defining a 3′ endpoint for each species, resulting in biotinylated double-stranded cDNA.

Each biotinylated double-stranded cDNA sample was cleaved with the restriction endonuclease MspI, which recognizes the sequence CCGG. The resulting fragments of cDNA corresponding to the 3′ region of the starting mRNA were then isolated by capture of the biotinylated cDNA fragments on a streptavidin-coated substrate. Suitable streptavidin-coated substrates include microtitre plates, PCR tubes, polystyrene beads, paramagnetic polymer beads, and paramagnetic porous glass particles. A preferred streptavidin-coated substrate is a suspension of paramagnetic polymer beads (Dynal, Inc., Great Neck, N.Y.).

After washing the streptavidin-coated substrate and captured biotinylated cDNA fragments, the cDNA fragment product was released by digestion with NotI, which cleaves at an 8-nucleotide sequence within the anchor primers but rarely within the mRNA-derived portion of the cDNAs. The 3′ MspI-NotI fragments, which are of uniform length for each mRNA species, were directionally ligated into ClaI- NotI-cleaved plasmid pBC SK+ (Stratagene, La Jolla, Calif.) in an antisense orientation with respect to the vector's T3 promoter, and the product used to transform Escherichia coli SURE cells (Stratagene). The ligation regenerates the NotI site, but not the MspI site, leaving CGG as the first 3 bases of the 5′ end of all PCR products obtained. Each library contained in excess of 5×10⁵ recombinants to ensure a high likelihood that the 3′ ends of all mRNAs with concentrations of 0.001% or greater were multiply represented. Plasmid preps (Qiagen) were made from the cDNA library of each sample under study.

An aliquot of each library was digested with MspI, which effects linearization by cleavage at several sites within the parent vector while leaving the 3′ cDNA inserts and their flanking sequences, including the T3 promoter, intact. The product was incubated with T3 RNA polymerase (MEGAscript kit, Ambion) to generate antisense cRNA transcripts of the cloned inserts containing known vector sequences abutting the MspI and NotI sites from the original cDNAs.

At this stage, each of the cRNA preparations was processed in a three-step fashion. In step one, 250 ng of cRNA was converted to first-strand cDNA using the 5′ RT primer (A-G-G-T-C-G-A-C-G-G-T-A-T-C-G-G, (SEQ ID NO: 21). In step two, 400 pg of cDNA product was used as PCR template in four separate reactions with each of the four 5′ PCR primers of the form G-G-T-C-G-A-C-G-G-T-A-T-C-G-G-N (SEQ ID NO: 22), each paired with a “universal” 3′ PCR primer G-A-G-C-T-C-C-A-C-C-G-C-G-G-T (SEQ ID NO: 23) to yield four sets of PCR reaction products (“N1 reaction products”).

In step three, the product of each subpool was further divided into 64 subsubpools (2 ng in 20 μl) for the second PCR reaction. This PCR reaction comprised adding 100 ng of the fluoresceinated “universal” 3′ PCR primer (SEQ ID NO: 23) conjugated to 6-FAM and 100 ng of the appropriate 5′ PCR primer of the form C-G-A-C-G-G-T-A-T-C-G-G-N-N-N-N (SEQ ID NO: 24), and using a program that included an annealing step at a temperature X slightly above the Tm of each 5′ PCR primer to minimize artifactual mispriming and promote high fidelity copying. Each polymerase chain reaction step was performed in the presence of TaqStart antibody (Clonetech).

The products (“N4 reaction products”) from the final polymerase chain reaction step for each of the tissue samples were resolved on a series of denaturing DNA sequencing gels using the automated ABI Prizm 377 sequencer. Data were collected using the-GeneScan software package (ABI) and normalized for amplitude and migration. Complete execution of this series of reactions generated 64 product subpools for each of the four pools established by the 5′ PCR primers of the first PCR reaction, for a total of 256 product subpools for the entire 5′ PCR primer set of the second PCR reaction.

The mRNA samples extracted from the striatum/nucleus accumbens of mice treated with 7.5 mg/kg of clozapine for the following durations: control (no clozapine), 45 minutes, 7 hours, 5 days, 12 days, and 14 days were analyzed. Table 1 is a summary of the expression levels of 496 mRNAs determined from cDNA. These cDNA molecules are identified by their digital address, that is, a partial 5′ terminus nucleotide sequence coupled with the length of the molecule, as well as the relative amount of the molecule produced at different time intervals after treatment. The 5′ terminus partial nucleotide sequence is determined by the recognition site for MspI (CC GG) and the nucleotide sequence of the parsing bases of the 5′ PCR primer used in the final PCR step. The digital address length of the fragment was determined by interpolation on a standard curve and, as such, may vary ±1-2 b.p. from the actual length as determined by sequencing.

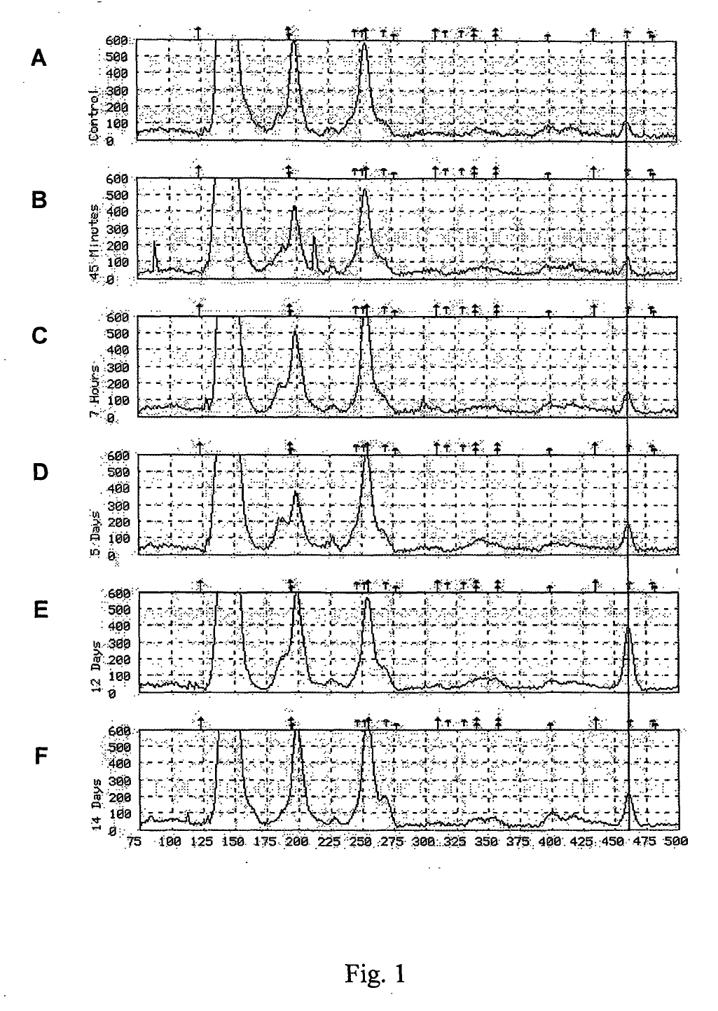

For example, the entry in Table 1 that describes a DNA molecule identified by the digital address MspI CTAA 461, is further characterized as having a 5′ terminus partial nucleotide sequence of CGGCTAA and a digital address length of 461 b.p. The DNA molecule identified as MspI CTAA 461 is further described as being expressed at increasing levels after 12 days of treatment with clozapine (see FIG. 1). Additionally, the DNA molecule identified as MspI CTAA 461 is described by its nucleotide sequence, which corresponds with SEQ ID NO: 37.

Similarly, the other DNA molecules identified in Table 1 by their MspI digital addresses are further characterized by: 1) the level of gene expression in the striatum/nucleus accumbens of mice without clozapine treatment (control), 2) the level of gene expression in the striatum/nucleus accumbens of mice treated with clozapine for 45 minutes, 3) the level of gene expression in the striatum/nucleus accumbens of mice treated with clozapine for 7 hours, 4) the level of gene expression in the striatum/nucleus accumbens of mice treated with clozapine for 5 days, 5) the level of gene expression in the striatum/nucleus accumbens of mice treated with clozapine for 12 days, 6) the level of gene expression in the striatum/nucleus accumbens of mice treated with clozapine for 14 days.

Additionally, several of the DSTs were further characterized as shown in the Tables and their nucleotide sequences are provided as SEQ ID NOs: 1-12; 14-19; 28-31; and 36-50 in the Sequence Listing below.

The ligation of the sequence into a vector does not regenerate the MspI site; the experimentally determined sequence reported herein has C-G-G as the first bases of the 5′ end.

The data shown in FIG. 1 were generated with a 5′-PCR primer (C-G-A-C-G-G-T-A-T-C-G-G-C-T-A-A; SEQ ID NO: 66) paired with the “universal” 3′ primer (SEQ ID NO:23) labeled with 6-carboxyfluorescein (6FAM, ABI) at the 5′ terminus. PCR reaction products were resolved by gel electrophoresis on 4.5% acrylamide gels and fluorescence data acquired on ABI377 automated sequencers. Data were analyzed using GeneScan software (Perkin-Elmer).

FIG. 1 is a graphical representation of the results of TOGA™ runs using a 5′ PCR primer with parsing bases CTAA (SEQ ID NO:66) and the universal 3′ PCR primer (SEQ ID NO:23) showing PCR products produced from mRNA extracted from the striatum/nucleus accumbens of mice treated with 7.5 mg/kg of clozapine for the following durations: control (no clozapine) (Panel A), 45 minutes (Panel B), 7 hours (Panel C), 5 days (Panel D), 12 days (Panel E), and 14 days (Panel F), where the vertical index line indicates a PCR product of about 461 b.p. that is expressed to a greater level in the 12 day clozapine-treated sample than in the other samples. The horizontal axis represents the number of base pairs of the molecules in these samples and the vertical axis represents the fluorescence measurement in the TOGA™ analysis (which corresponds to the relative expression of the molecule of that address). The results of the TOGA™ runs have been normalized using the methods described in pending U.S. patent application Ser. No. 09/318,699/U.S., and pending PCT Application Serial No. PCT/US00/14159, both entitled Methods and System for Amplitude Normalization and Selection of Data Peaks (Dennis Grace, Jayson Durham); and pending U.S. patent application Ser. No. 09/318,679/U.S. and pending PCT Application Serial No. PCT/US00/14123, both entitled Methods for Normalization of Experimental Data (Dennis Grace, Jayson Durham) all of which are incorporated herein by reference. The vertical line drawn through the five panels represents the DST molecule identified as CLZ_{—}43 (SEQ ID NO:37).

Some products, which were differentially represented, appeared to migrate in positions that suggest that the products were novel based on comparison to data extracted from GenBank. The sequences of such products were determined by one of two methods: cloning or direct sequencing of the PCR products.

Cloning of TOGA™ Generated PCR Products

In suitable cases, the PCR product was isolated, cloned into a TOPO vector (Invitrogen) and sequenced on both strands. The database matches for each cloned DST sequence are listed in Table 2. CLZ_{—}43 (SEQ ID NO:37), the DNA molecule identifed by MspI CTAA 461, was one such cloned product. In order to verify that the cloned product corresponds to the TOGA™ peak of interest, the extended TOGA™ assay was performed for each DST (see below).

Direct Sequencing of TOGA™ Generated PCR Products

In other cases, the TOGA™ PCR product was sequenced using a modification of a direct sequencing methodology (Innis et al., Proc. Nat'l. Acad. Sci., 85: 9436-9440 (1988)).

PCR products corresponding to DSTs were gel purified and PCR amplified again to incorporate sequencing primers at 5′ and 3′ ends. The sequence addition was accomplished through 5′ and 3′ ds-primers containing M13 sequencing primer sequences (M13 forward and M13 reverse respectively) at their 5′ ends, followed by a linker sequence and a sequence complementary to the DST ends. Using the Clontech Taq Start antibody system, a master mix containing all components except the gel purified PCR product template was prepared, which contained sterile H₂O, 10× PCR II buffer, 10 mM dNTP, 25 mM MgCl₂, AmpliTaq/Antibody mix (1.1 μg/μl Taq antibody, 5 U/μl AmpliTaq), 100 ng/μl of 5′ ds-primer (5′ TCC CAG TCA CGA CGT TGT AAA ACG ACG GCT CAT ATG AAT TAG GTG ACC GAC GGT ATC GG 3′, SEQ ID NO: 52), and 100 ng/μl of 3′ ds-primer (5′ CAG CGG ATA ACA ATT TCA CAC AGG GAG CTC CAC CGC GGT GGC GGC C 3′, SEQ ID NO: 53). After addition of the PCR template, PCR was performed using the following program: 94° C., 4 minutes and 25 cycles of 94° C, 20 seconds; 65° C., 20 seconds; 72° C., 20 seconds; and 72° C. 4 minutes. The resulting amplified adapted PCR product was gel purified.

The purified PCR product was sequenced using a standard protocol for ABI 3700 sequencing. Briefly, triplicate reactions in forward and reverse orientation (6 total reactions) were prepared, each reaction containing 5 μl of gel purified PCR product as template. In addition, the sequencing reactions contained 2 μl 2.5× sequencing buffer, 2 μl Big Dye Terminator mix, 1 μl of either the 5′ sequencing primer (5′ CCC AGT CAC GAC GTT GTA AAA CG 3′, SEQ ID NO: 54), or the 3′ sequencing primer (5′ TTT TTT TTT TTT TTT TTT V 3′, where V=A, C, or G, SEQ ID NO: 55) in a total volume of 10 μl.

In an alternate embodiment, the 3′ sequencing primer was the sequence 5′ GGT GGC GGC CGC AGG AAT TTT TTT TTT TTT TTT 3′, (SEQ ID NO: 56). PCR was performed using the following thermal cycling program: 96° C., 2 minutes and 29 cycles of 96° C., 15 seconds; 50° C., 15 seconds; 60° C., 4 minutes.

The sequences for (CLZ_{—}62, SEQ ID NO: 49 and CLZ_{—}65, SEQ ID NO: 50) were determined by this method. Table 2 contains the database matches for the sequences determined by this method. In order to verify that the product determined by direct sequencing corresponds to the TOGA™ peak of interest, the extended TOGA™ assay was performed for each DST (see below).

Verification Using the Extended TOGA™ Method

In order to verify that the TOGA™ peak of interest corresponds to the identified DST, an extended TOGA™ assay was performed for each DST as described below. PCR primers (“Extended TOGA™ primers”) were designed from sequence determined using one of three methods: (1) in suitable cases, the PCR product was isolated, cloned into a TOPO vector (Invitrogen) and sequenced on both strands; (2) in other cases, the TOGA™ PCR product was sequenced using a modification of a direct sequencing methodology (Innis et al., Proc. Nat'l. Acad. Sci., 85: 9436-9440 (1988)) or (3) in many cases, the sequences listed for the TOGA™ PCR products were derived from candidate matches to sequences present in available GenBank, EST, or proprietary databases.

PCR was performed using the Extended TOGA™ primers and the N1 PCR reaction products as a substrate. Oligonucleotides were synthesized with the sequence G-A-T-C-G-A-A-T-C extended at the 3′ end with a partial MspI site (C-G-G), and an additional 18 adjacent nucleotides from the determined sequence of the DST. For example, for the PCR product with the TOGA™ address CTAA 461 (CLZ_{—}43; SEQ ID NO:37), the 5′ PCR primer was G-A-T-C-G-A-A-T-C-C-G-G-C-T-A-A-T-A-T-T-G-A-T-A-A-T-C-T-T-T (SEQ ID NO:72). This 5′ PCR primer was paired with the fluorescence labeled universal 3′ PCR primer (SEQ ID NO:23) in a PCR reaction using the PCR N1 reaction product as substrate.

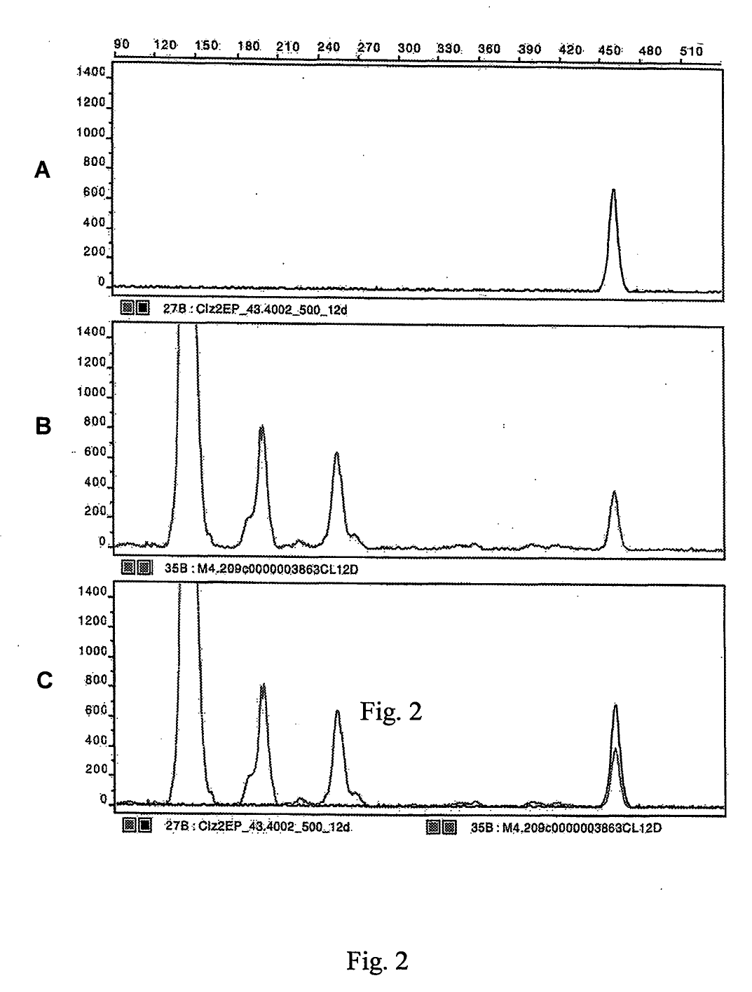

The length of the PCR product generated with the Extended TOGA™ primer was compared to the length of the original PCR product that was produced in the TOGA™ reaction. The results for SEQ ID NO:37, for example, are shown in FIG. 2. The length of the PCR product corresponding to SEQ ID NO:37 (CLZ_{—}43) was cloned and a 5′ PCR primer was built from the cloned-DST (SEQ ID NO:72). The product obtained from PCR with this primer (SEQ ID NO:72) and the universal 3′ PCR primer (SEQ ID NO:23) (as shown in the top panel) was compared to the length of the original PCR product that was produced in the TOGA™ reaction with mRNA extracted from the striatum/nucleus accumbens of mice treated with 7.5 mg/kg of clozapine for 12 days using a 5′ PCR primer with parsing bases CTAA (SEQ ID NO:66) and the universal 3′ PCR primer (SEQ ID NO:23) (as shown in the middle panel). Again, for all panels, the number of base pairs is shown on the horizontal axis, and fluorescence intensity (which corresponds to relative expression) is found on the vertical axis. In the bottom panel, the traces from the top and middle panels are overlaid, demonstrating that the peak found using an extended primer from the cloned DST is the same number of base pairs as the original PCR product obtained through TOGA™ as CLZ_{—}43 (SEQ ID NO:37). The bottom panel thus illustrates that CLZ_{—}43 (SEQ ID NO:37) was the DST amplified in Extended TOGA™. The same method was used to verify that the sequences determined by direct sequencing derive from the PCR product of interest.

In four cases, CLZ_{—}17, (SEQ ID NO: 28); CLZ_{—}26, (SEQ ID NO: 29); CLZ 28, (SEQ ID NO: 30); and CLZ_{—}58 (SEQ ID NO: 31) the sequences listed for the TOGA™ PCR products were derived from candidate matches to sequences present in available Genbank, EST, or proprietary databases. Table 3 lists the candidate matches for each by accession number of the Genbank entry or by the accession numbers of a set of computer-assembled ESTs used to create a consensus sequence. Extended TOGA™ primers were designed based on these sequences (as mentined previously), and Extended TOGA™ was run to determine if the database sequences were the DSTs amplified in TOGA™. The sequences that these DSTs were based on are found in the Sequence listing as SEQ ID NOs:32-35.

Sequence Identification of DSTs

A preferred method for determining the best overall match between a query sequence (a sequence of the present invention) and a subject sequence, also referred to as a sequence database, can be determined using the BLAST computer program based on the algorithm of Altschul and colleagues (Altschul, S. F., Gish, W., Miller, W., Myers, E. W. & Lipman, D. J. (1990), “Basic local alignment search tool.” J. Mol. Biol. 215:403-410; Altschul, S. F., Madden, T. L., Schäffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402.). The term “sequence” includes nucleotide and amino acid sequences. In a sequence alignment, the query sequence can be either protein or nucleic acid or any combination therein. BLAST is a statistically driven search method that finds regions of similarity between a query and database sequences. These are called segment pairs, and consist of gapless alignments of any part of two sequences. Within these aligned regions, the sum of the scoring matrix values of their constituent symbol pairs is higher than a level expected to occur by chance alone. The scores obtained in a BLAST search can be interpreted by the experienced investigator to determine real relationships versus random similarities. The BLAST program supports four different search mechanisms:
Nucleotide Query Searching a Nucleotide Database—Each database sequence is compared to the query in a separate nucleotide-nucleotide pairwise comparison.
Protein Query Searching a Protein Database—Each database sequence is compared to the query in a separate protein-protein pairwise comparison.
Nucleotide Query Searching a Protein Database—The query is translated, and each of the six products is compared to each database sequence in a separate protein-protein pairwise comparison.
Protein Query Searching a Nucleotide Database—Each nucleotide database sequence is translated, and each of the six products is compared to the query in a separate protein-protein pairwise comparison.

By using the BLAST program to search for matches between a sequence of the present invention and sequences in GenBank and EST databases, identities were assigned whenever possible. A portion of these results are listed in Table 2.

DST Validation Using Real-Time Quantitative PCR

Validation of the DST expression for select DSTs was done by Real Time PCR using the ABI PRISM 7700 Sequence Detection System (PE Biosystems) that combines PCR, cycle-by-cycle fluorescence detection and analysis software for high-throughput quantitation of nucleic acid sequences. Using the SYBR Green Reagent as the fluorescent report molecule, direct detection of the PCR product was monitored by measuring the increase in fluorescence caused by the binding of SYBR Green to double-stranded DNA. Reactions are characterized by the point in time when amplification of a PCR product is first detected rather than the amount of PCR product accumulated after a fixed number of cycles. The higher the copy number of the nucleic acid target, the earlier a significant increase in fluorescence is observed. Quantitation of the amount of target in the sample is accomplished by measuring the cycle number at which a significant amount of product is produced. The entire process is performed by the integrated software of the 7700 system. Primers for the Real Time PCR validation are selected by the integrated software package Primer Express) accompanying the ABI PRISM 7700. Standards for normalizing the quantitation of gene levels were chosen from a panel of 5 mouse “housekeeping” genes. The normalization standard chosen was cyclophilin and was based on the similarity of expression across all sample templates.

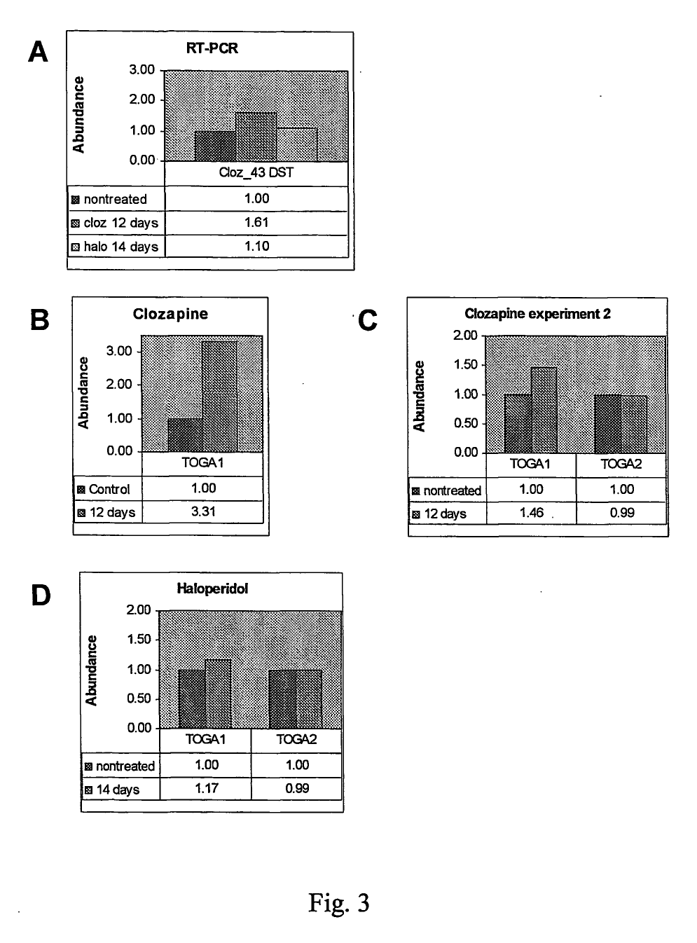

FIG. 3A-D compares the results from Real Time PCR validation (A) (as described below) to the TOGA™ result from three different experiments: the original clozapine experiment (B), a repeated clozapine experiment performed in duplicate (C), and a haloperidol experiment in duplicate (D). As the tables and graphs illustrate, TOGA™ and Real Time PCR show that the DST CLZ_{—}43 (SEQ ID NO:37) increases in expression in clozapine treated mice, while is not responsive to haloperidol treatment. Table 5 lists the 5′ and 3′ primers used in these Real-Time PCR studies. Similar experiments were performed for DST CLZ_{—}40 (SEQ ID NO:12) and CLZ_{—}5 (SEQ ID NO:2).

### EXAMPLE 2

### Characterization of CLZ 43

Male C57B1/6J mice (20-28 g) were housed as previously described in Example 1. The same experimental paradigm used in Example 1 for clozapine treatment was used for the various analyses described below. Briefly, in the clozapine studies, the control group mice received a single injection of sterile saline (0.1 ml volume), or no injection, and were sacrificed after 45 minutes. The mice subjected to acute clozapine treatment were given a single intraperitoneal injection of clozapine (7.5 mg/kg) and sacrificed after 45 minutes or 7 hours, as described in Example 1. The mice subjected to chronic clozapine treatment received daily subcutaneous injections of clozapine (7.5 mg/kg) for 5 days, 12 days or 14 days. All animals were sacrificed in their cages with C0₂ at the indicated times. Brains were rapidly removed and placed on ice. The striatum, including the nucleus accumbens, were dissected out and placed in ice-cold phosphate-buffered saline. The mRNA was prepared according to the method described in the Example 1.

The TOGA™ data shown in FIG. 1 was generated with a 5′-PCR primer (C-G-A-C-G-G-T-A-T-C-G-G-C-T-A-A; SEQ ID NO:66) paired with the “universal” 3′ primer (SEQ ID NO:23) labeled with 6-carboxyfluorescein (6FAM, ABI) at the 5′ terminus. PCR reaction products were resolved by gel electrophoresis, fluorescence data acquired on ABI377 automated sequencers, and data were analyzed using GeneScan software (Perkin-Elmer) as described in Example 1. FIG. 2 presents a graphical example of the results obtained when a DST is verified by the Extended TOGA™ method using a primer generated from a cloned product (as described in Example 1).

As shown in Table 1, the results of TOGA™ analysis indicate that CLZ_{—}43 (SEQ ID NO:37) is up-regulated by 12 days of clozapine treatment. Table 2 shows that CLZ_{—}43 matches an EST isolated from mouse tissue. Several mouse ESTs demonstrated high similarity to the DST and homology was also found with a human 5556 b.p. GenBank entry (AB040884, also known as KIAA1451). BLAST analysis of the human protein sequence revealed several yeast homologs that comprise a family of oxysterol binding protein related sequences. The same BLAST analysis also revealed additional oxysterol binding protein-related sequences in a diversity of species (e.g., yeast, human, mouse, rabbit, C. elegans, Drosophila, Neurospora, Arabidopsis). Thus, it is believed that CLZ_{—}43 is a novel member of a family of oxysterol-binding proteins.

Validation of CLZ_{—}43 expression was done by using Real Time PCR as described in Example 1. FIG. 3A-D compares the results from Real Time PCR validation (A) to the TOGA™ result from three different experiments: the original clozapine experiment (B), a repeated clozapine experiment performed in duplicate (C), and a haloperidol experiment in duplicate (D). As the tables and graphs illustrate, TOGA™ and Real Time PCR show that the DST CLZ_{—}43 (SEQ ID NO:37) increases in expression in mice treated with clozapine for 12 days, while is not significantly responsive to haloperidol treatment for 14 days, relative to untreated mice. Table 5 lists the 5′ and 3′ primers used in these Real-Time PCR studies. Thus, Real Time PCR confirmed that TOGA™ predicted a unique pattern by these two neuroleptics.

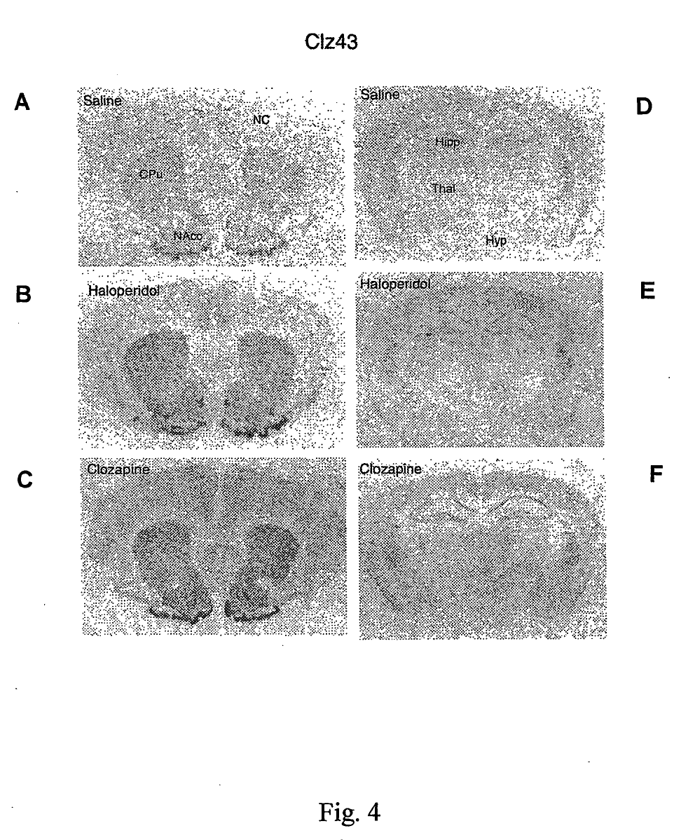

In further characterization of CLZ_{—}43, in situ hybridization analysis using an antisense cRNA probe directed against the 3′ end of CLZ_{—}43 were performed to show the pattern of CLZ_{—}43 mRNA expression (FIG. 4A-F). In situ hybridization was performed on free-floating coronal sections (25 μM thick) with an ³⁵S-labeled, single-stranded antisense cRNA probe of CLZ_{—}43. Coronal sections were hybridized at 55° C for 16 hour with an ³⁵S-labeled, single-stranded antisense cRNA probe of CLZ_{—}43 at 10⁷ cpm/ml. The probe was synthesized from the 3′-ended cDNA TOGA™ clone using the Maxiscript Transcription Kit (Ambion, Austin, Tex.). Excess probe was removed by washing with 2×SSC (I×SSC=0.015 M NaCl/0.0015 M Na citrate) containing 14 mM β-mercaptoethanol (30 minutes), followed by incubation with 4 μg/ml ribonuclease in 0.5 M NaCl/0.05 M EDTA/0.05 M Tris-HCl, pH 7.5, for 1 hour at 37° C. High stringency washes were carried out at 55° C. for 2 hours in 0.5×SSC/50% formamide/0.01 M β-mercaptoethanol, and then at 68° C. for 1 hour in 0.1×SSC/0.01 M β-mercaptoethanol/0.5% sarkosyl. Slices were mounted onto gelatin-coated slides and dehydrated with ethanol and chloroform before autoradiography. Slides were exposed for 1-4 days to Kodak X-AR film and then dipped in Ilford K-5 emulsion. After 4 weeks, slides were developed with Kodak D19 developer, fixed, and counterstained with Richardson's blue stain.

FIG. 4A-F demonstrates the pattern of CLZ_{—}43 mRNA expression in coronal sections where A, B and C were sectioned at the level of the striatum (containing nucleus accumbens, Nacc, caudateputamen, Cpu, and neocortex, NC) and D, E, and F were sectioned at the level of the thalamus (Thal), hippocampus (Hipp), and hypothalamus (Hyp). A low level of expression was observed in the striatum, and treatment with either haloperidol or clozapine resulted in increased expression in the neocortex and in the striatum in mouse brain (B and C). Comparison with brain sections obtained from control mice showed that CLZ_{—}43 expression is increased by chronic treatment (2 weeks) with clozapine (˜10-fold) or haloperidol (˜3-fold). Thus, although haloperidol did not appear to induce expression of CLZ_{—}43 by TOGA™ or by Real Time PCR, a highly localized pattern of expression was observed by in situ hybridization that was similar in pattern to that caused by clozapine. This common expression pattern suggests this DST is a strong candidate for an RNA encoding protein whose activity is involved in the benefit derived by patients from these two classes of neuroleptic drugs.

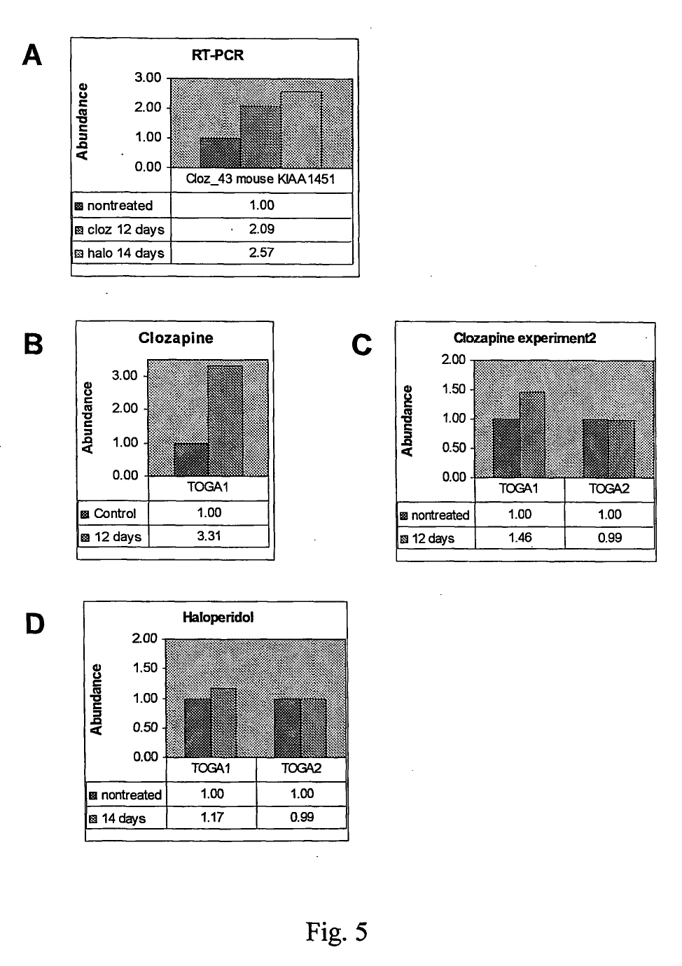

As described above, homology was found with a human 5556 b.p. GenBank entry (AB040884, also known as KIAA1451). To obtain the homologous mouse sequence corresponding to the human KIAA1451, primers were designed to the 3′ region of the human KIAA1451 coding region and used to amplify a 336 bp PCR product from a cDNA preparation made from mouse whole brain (SEQ ID NO: 79). PCR products were gel purified, cloned into the TOPO plasmid vector (pet100D TOPO, Invitrogen) and sequenced on both strands. Nucleotide sequences were determined by standard techniques. In order to verify that the cloned PCR product corresponds to the sequence of interest, sequences were aligned and assembled into contigs using the DNA alignment program SeqMan (DNAStar). FIG. 5A-D compares the results from Real Time PCR validation (A) (as described in Example 1) to the TOGA™ result from three different experiments: the original clozapine experiment (B), a repeated clozapine experiment performed in duplicate (C), and a haloperidol experiment in duplicate (D). As the tables and graphs illustrate, the Real Time PCR shows that the mouse sequence homolog to human KIAA1451 (SEQ ID NO:79) increases in expression in mice chronically treated with clozapine (2.09-fold) or haloperidol (2.57-fold). Due to the different Real Time PCR profile (2.09-fold increase) compared to TOGA™ profile (average 1.08-fold) for the haloperidol response (i.e., not regulated), it is believed that the mouse KIAA1451-related sequence represents a neuroleptic responsive gene that is related, but distinct from the DST CLZ_{—}43. This sequence also contains similarity to the same oxysterol binding protein family member as the DST.

In order, to obtain further information about the human KIAA1451 sequence, an oligonucleotide designed from the human KIAA1451 sequence was used to isolate the remaining 5′end of the human gene from an adult human brain cDNA plasmid library. The target pool was a cDNA plasmid library created from adult human brain RNA. The oligonucleotide sequence used for hybridization was 5′—AAC AAG TCC GTC CTG GCA TGG-3′ (SEQ ID NO:5 1). The clone was isolated using the methods prescribed by the manufacturer of the GeneTrapper kit (Life Technologies, Inc.). The capture oligonucleotides was end-labelled with biotin-14-dCTP using terminal deoxynucloetidyl transferase and the cDNA plasmid pool was converted from double-stranded cDNA to single-stranded cDNA through the specific action of GeneII protein and exonuclease III. The single-stranded cDNA pool was combined with the end-labelled oligonucleotides, hybridized, mixed with strepavidin-coated magnetic beads, and plasmid/oligonucleotide hybrids were purified by magnetic separation. The single-stranded plasmid DNA was released from the oligonucleotide and repaired back into a double-stranded plasmid using a fresh sample of the capture oligonucleotide and DNA polymerase. Plasmid DNA was prepared from bacteria transformed with the repaired plasmids and subjected to sequence analysis. Using this methodology, a 1717 b.p. cDNA clone (SEQ ID NO:68) was isolated that overlaps with the known human KIAA1451 sequence. This clone provides an additional (novel) 512 b.p. at the 5′end of the GenBank entry. Sequence analysis suggests the position of the methionine start codon for the open reading frame is at base 562 of the 1717 b.p. clone (SEQ ID NO: 69). The open reading of the 1717 b.p. clone encodes a 385 amino acid peptide (SEQ ID NO: 69, SEQ ID NO: 70). Homology matches with a human genome database have identified 7 exons spread across more than 22,000 b.p. Further it has been determined that within this cluster is a region that has been mapped to chromosome 12, which is not a chromosome previously linked to schizophrenia. The sequence data reveals that the open reading frame encodes a protein of 472 amino acids (SEQ ID NO: 71). Comparison with protein databases indicates that this novel (putative) protein is clearly a member of a class of proteins that binds lipids, especially oxysterols. Due to the similarity of the nucleic acid sequences for the mouse homolog to the human KIAA1451 sequence (SEQ ID NO:79), the human KIAA1451 sequence (SEQ ID NO:68), and the DST CLZ_{—}43 (SEQ ID NO:37), it is believed that these all represent novel members of a class of oxysterol binding protein.

The observation that, of thousands of proteins expressed by the striatum, a novel oxysterol binding protein and apoD (see CLZ_{—}5, Example 4 below) are among the few modulated by neuroleptic drugs strengthens the hypothesis that schizophrenia is a disease of brain sterol homeostasis, and thus may have etiologies as diverse as atherosclerosis. The brain has by far more cholesterol and 24S-hydroxysterol than any organ other than the adrenal glands, and the special importance of the membrane activities of neurons and their myelinating cells are self-evident. The lipid bilayer of the membrane is made up of glycerolphopholipids and cholesterol, and variations in composition and hydrocarbon chain saturation state determine membrane order and fluidity. These properties affect the binding of extrinsic membrane proteins and, thus, second messenger signaling. As we have shown previously, a large percentage of the mRNAs highly enriched in the striatum encode proteins that regulate second messenger signaling along the inner membrane. Thus, a panneural or panorganismic disruption in lipid metabolism might manifest first as a striatal disease.

### EXAMPLE 3

### Characterization of CLZ 40

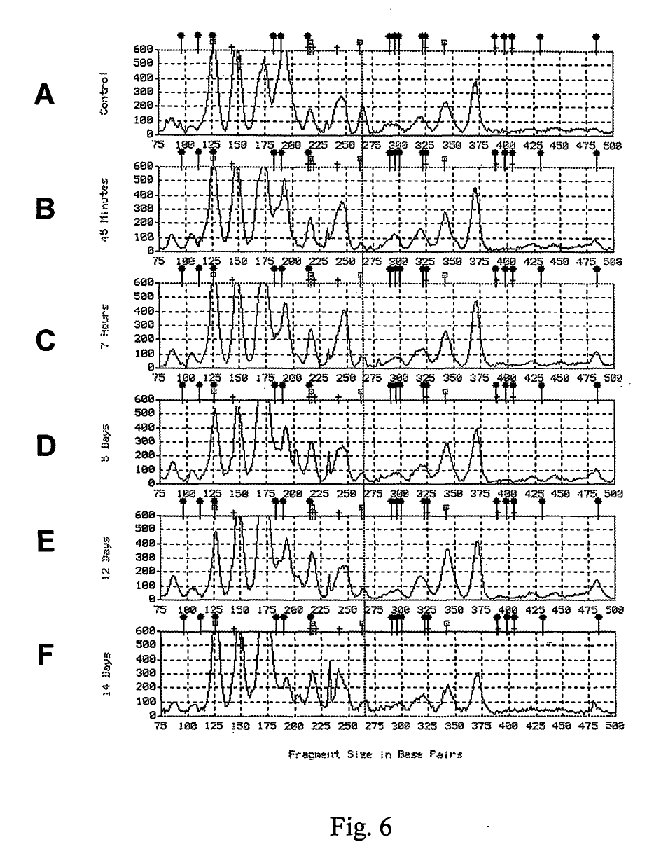

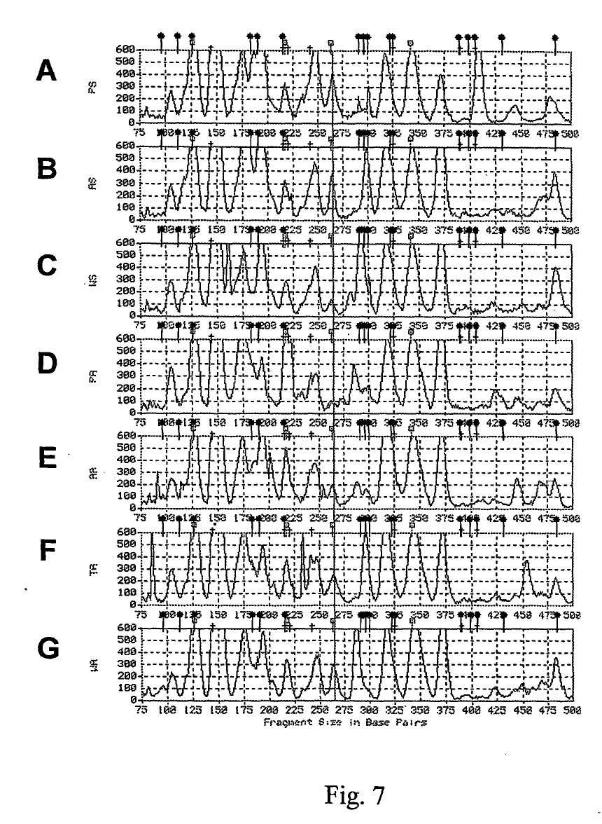

Animals were treated with clozapine and the mRNA was prepared according to the method described in Example 1. The TOGA™ data shown in FIG. 6 was generated with a 5′-PCR primer (C-G-A-C-G-G-T-A-T-C-G-G-T-T-G-T; SEQ ID NO: 26) paired with the “universal” 3′ primer (SEQ ID NO: 23) labeled with 6-carboxyfluorescein (6FAM, ABI) at the 5′ terminus. PCR reaction products were resolved by gel electrophoresis on 4.5% acrylamide gels and fluorescence data acquired on ABI377 automated sequencers. Data were analyzed using GeneScan software (Perkin-Elmer). In addition, this primer was shown to produce a DST that appeared regulated in mice treated with morphine (FIG. 7).

For the morphine studies, male C57B1/6J mice (20-28 g) were housed as previously described in Example 1 and divided into the following groups: 1) a control group, in which the mice were subcutaneusly implanted with one placebo pellet upon halothane anaesthesia; 2) an acute morphine group, in which the mice received a morphine intraperitoneal injection of 10 mg/kg; 3) a chronic or tolerant group, in which mice were rendered drug-tolerant and dependent by means of subcutaneous implantation of a single pellet containing 75 mg of morphine free base for 3 days; and 4) a withdrawal group, in which the mice rendered tolerant to morphine were injected intraperitoneally with naltrexone 1 mg/kg. Animals were sacrificed in their cages with CO₂ at 72 hours after placebo or morphine pellet implantation, or 4 hours after single injection of morphine, or 4 hours after administration of naltrexone to morphine-tolerant mice. Their brains were rapidly removed. The striatum, including the nucleus accumbens, and block of tissues containing the amygdala complex were dissected under microscope and collected in ice-cold RNA extraction buffer.

The results of TOGA™ analysis using a 5 PCR primer with parsing bases C-G-A-C-G-G-T-A-T-C-G-G-T-T-G-T (SEQ ID NO: 26) and the universal 3′ primer (SEQ ID NO:23) are shown in FIGS. 6 and 7, which show PCR products produced from mRNA isolated from the striatum/nucleus accumbens of mice treated with clozapine (FIG. 6) or morphine (FIG. 7). In FIG. 6, the vertical index line indicates a PCR product of about 266 b.p. that is present in control cells, and whose expression decreases in the striatum/nucleus accumbens of mice treated with clozapine for 45 minutes, 7 hours, 5 days, 12 days, and 14 days. The down-regulation of CLZ_{—}40 (SEQ ID NO: 12) occurs as early as 45 minutes following clozapine treatment and remains downregulated for at least 14 days.

In FIG. 7, the vertical index line indicates a PCR product of about 266 b.p. that is present in control cells, and whose expression differentially regulated in control striatum (PS), acutely treated striatum (AS), withdrawal striatum (WS), control amygdala (PA), acutely treated amygdala (AA), chronically treated amygdala (TA), and withdrawal amygdala (WA). The expression of CLZ_{—}40 product is greater in striatum than in amygdala. Further, CLZ_{—}40 displays chronic-specific or withdrawal-specific regulation in both of these brain regions. In striatum, CLZ_{—}40 is downregulated in withdrawal striatum but not acutely treated striatum. In amygdala, CLZ_{—}40 is slightly upregulated in acutely treated amygdala and increasingly upregulated in chronically treated amygdala and withdrawal amygdala.

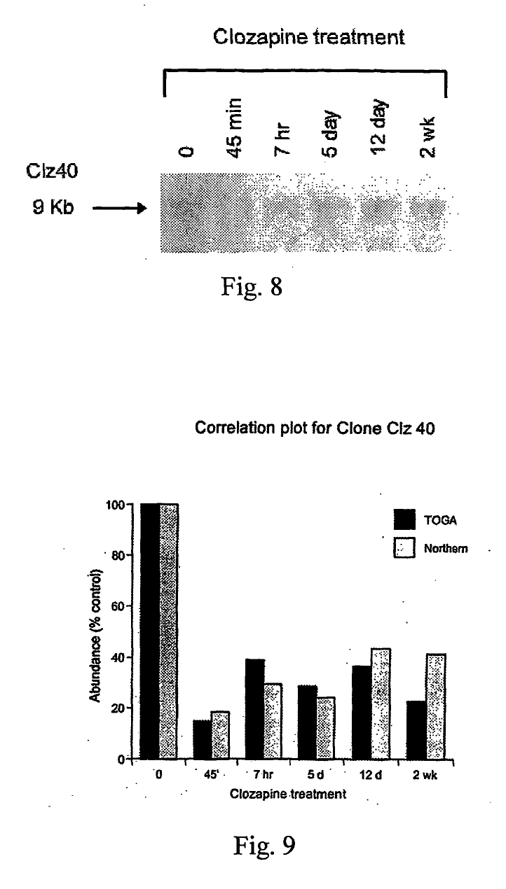

Shown in FIG. 8, Northern Blot analysis was performed using mRNA extracted from the striatum/nucleus accumbens of control mice and clozapine-treated mice. Briefly, an agarose gel containing 2 μg of poly A enriched mRNA as well as size standards was electrophoresed on a 1.5% agarose gel containing formaldehyde, transferred to a biotrans membrane, and prehybridized for 30 minutes in Expresshyb (Clonetech). An 265 bp insert of CLZ_{—}40 (25-100 ng) was labeled with [α-³²P]-d CTP by oligonucleotide labeling to specific activities of approximately 5×10⁸ cpm/μg and added to the prehybridization solution and incubated 1 hour. Filters were washed to high stringency (0.2×SSC) (1×SSC: 0.015 M NaCl and 0.0015 M Na citrate) at 68° C. then exposed to Kodak X-AR film for up to 1 week. As shown in FIG. 8, an approzimately 9 Kb transcript was detected in control and clozapine-treated mice which decreases dramatically after 45 minutes with clozapine treatment and remains down-regulated for at least 14 days. Table 4A contains a summary of these Northern results.

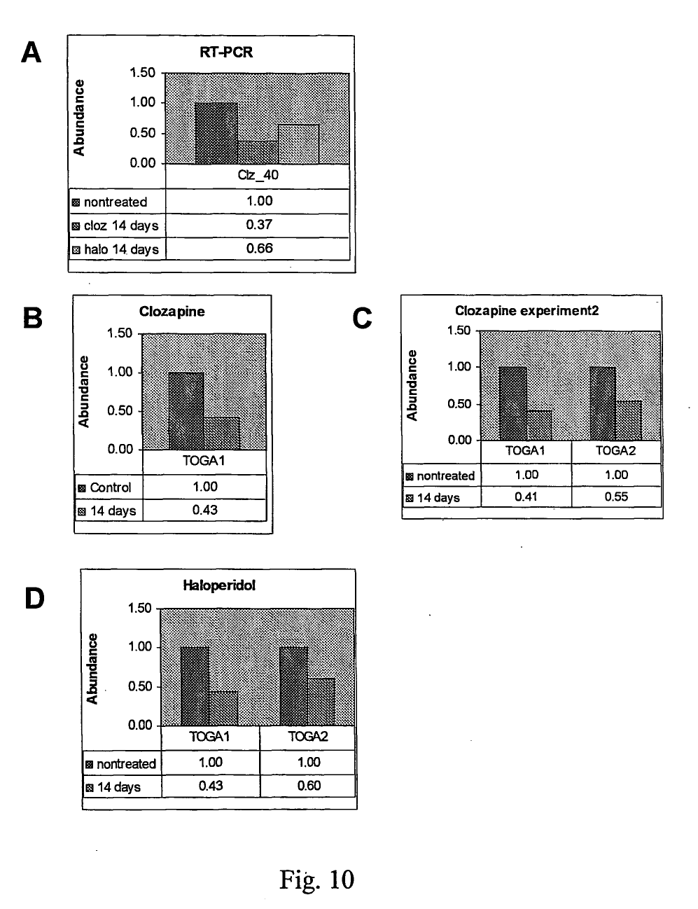

FIG. 9 is a graphical representation comparing the results of the clozapine treatment TOGA™ analysis of clone CLZ_{—}40 shown in FIG. 6 and the clozapine treatment Northern Blot analysis of clone CLZ_{—}40 shown in FIG. 8. The Northern Blot was imaged using a phosphorimager to determine the amount of CLZ_{—}40 mRNA in each clozapine-treated sample relative to the amount of mRNA in the control sample. As can be seen, the clozapine treatment TOGA™ analysis shows correlation with the clozapine treatment Northern Blot analysis. The single transcript of approximately 9 Kb was decreased in abundance after 45 minutes and 7 hr of clozapine treatment, consistent with TOGA.

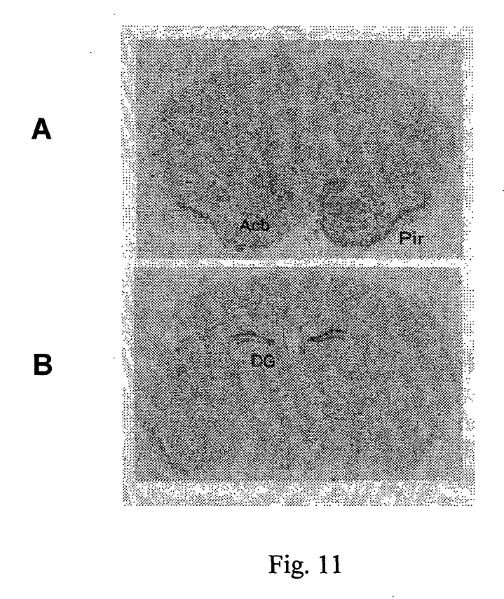

CLZ_{—}40 was further validated by Real Time PCR using cDNA from mice chronically (2 weeks) treated with clozapine or haloperidol (FIG. 10A-D). FIG. 10A-D compares the Real Time PCR data (A) to the TOGA™ result from three different experiments: the original clozapine experiment (B), a repeated clozapine experiment performed in duplicate (C), and a haloperidol experiment in duplicate (D). Real Time PCR analysis demonstrated a decrease of 0.37-fold in expression in clozapine treated mice and 0.66-fold decrease in haloperidol treated mice. This common expression pattern suggests this DST is a strong candidate for an RNA encoding protein whose activity is involved in the benefit derived by patients from these two classes of neuroleptic drugs. Table 5 lists the 5′ and 3′ primers used in these Real-Time PCR studies.

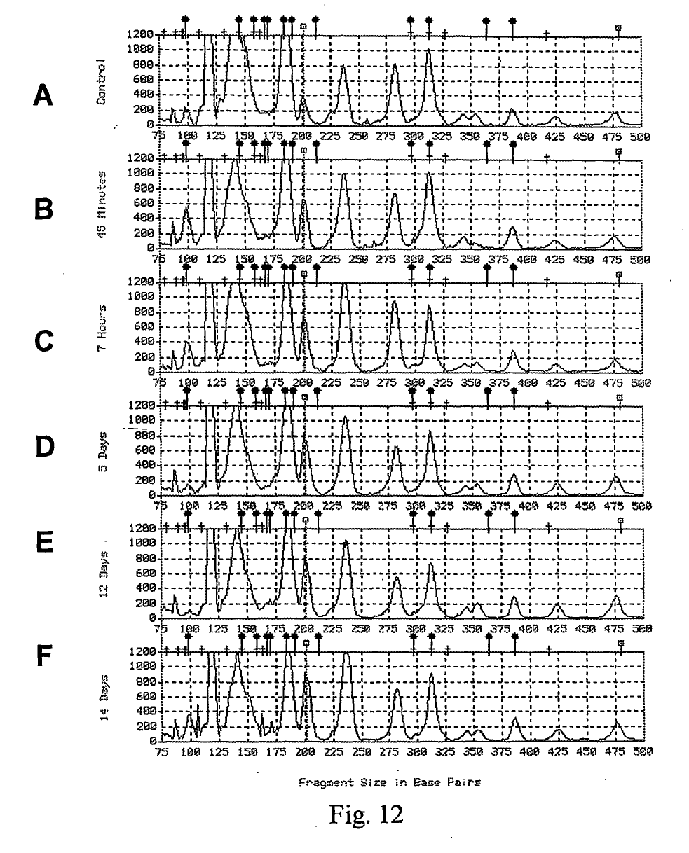

FIG. 11A-B shows in situ hybridization analysis, demonstrating CLZ_{—}40 mRNA expression in the mouse brain. In situ hybridization was performed on free-floating sections as described in Example 2. Interestingly, CLZ_{—}40 mRNA is specifically expressed in the nucleus accumbens and pyriform cortex (FIG. 11A), and dentate gyrus (FIG. 11B), but is not detected in any other brain regions.

At present, CLZ_{—}40 (SEQ ID NO: 12) is of unknown identity. However, the CLZ_{—}40 DST has been PCR amplified and a larger cDNA clone that is approximately 1 Kb in length was obtained (SEQ ID NO:13). This sequence does match an EST in the Genbank database (AI509550) as shown in Table 4B. A modified solid phase format of RACE (rapid amplification of cDNA ends) from mouse striatum cDNA was also utilized to obtain a 652 b.p. sequence (SEQ ID NO:80).

The observation that CLZ_{—}40 is down-regulated with clozapine treatment suggests a potential association with the therapeutic effects of clozapine. Furthermore, its highly unique gene expression pattern is like no other gene identified to date, and its presence in the nucleus accumbens may implicate CLZ_{—}40 in a number of functional roles associated with this structure, namely limbic/mental behavior and addiction.

Addiction to opiates and other drugs of abuse is a chronic disease of the brain, most likely resulting from molecular and cellular adaptations of specific neurons to repeated exposure to opiates (Leshner, A., Science, 278, 45-47 (1997)). An important neural substrate implicated in the opioid reinforcement and addiction is the mesolimbic system, notably the nucleus accumbens (Everitt, et al, Ann. N.Y. Acad. Sci., 877, 412-438 (1999)). All highly addictive drugs, such as opiates, cocaine and amphetamines, produce adaptations in the neural circuitry of the nucleus accumbens, but the precise relationships are unknown. The molecular neuroadaptation which takes place in this structure may offer important insight into the mechanisms of drug addiction. CLZ_{—}40 is a likely candidate for involvement in such mechanisms due to its specific expression in the nucleus accumbens. Elucidation of the biology underlying psychoses and addiction is key to understanding the underlying causes of such disorders and may lead to the development of more effective treatments, including anti-addiction medications.

Furthermore, the behavioral mechanisms associated with addiction reflect mechanisms of learning and memory (White, N., Addiction, 91, 921-949 (1996)). The hippocampal system has long been associated with learning and memory, including forms of conditional associative learning (Sziklas, et al., Hippocampus, 8, 131-137 (1998)), which is the form of learning associated with addiction (Di Chiara, et al., Ann. N.Y Acad. Sci., 877,461-85 (1999)). The expression of CLZ_{—}40 in the hippocampus suggests that this gene may provide a link with such learning processes.

### EXAMPLE 4

### Characterization of CLZ 5 (apoD)

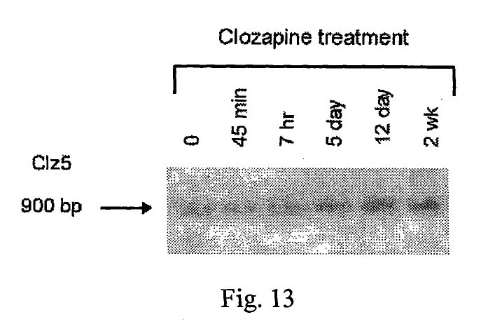

Animals were treated with clozapine and the mRNA was prepared according to the method described in Example 1. The result of TOGA™ analysis using a 5′ PCR primer with parsing bases C-G-A-C-G-G-T-A-T-C-G-G-C-A-C-C (SEQ ID NO:25) and the universal 3′ primer (SEQ ID NO:23) is shown in FIG. 12, which shows PCR products produced from mRNA isolated from the striatum/nucleus accumbens of mice treated with clozapine for various lengths of time as described in Example 1. The vertical index line indicates a PCR product of about 201 b.p. that is present in control cells, and whose expression increases when the striatum/nucleus accumbens of mice are treated with clozapine for 45 minutes, 7 hours, 5 days, 12 days, and 14 days.

As shown in Table 2, the CLZ_{—}5 clone (CACC 201; SEQ ID NO:2) corresponds with GenBank sequence X82648, which is identified as a mouse apolipoprotein D (apoD) sequence. Other corresponding apoD GenBank sequences include L39123 (mouse), X55572 (rat), NM_{—}001647 (human).

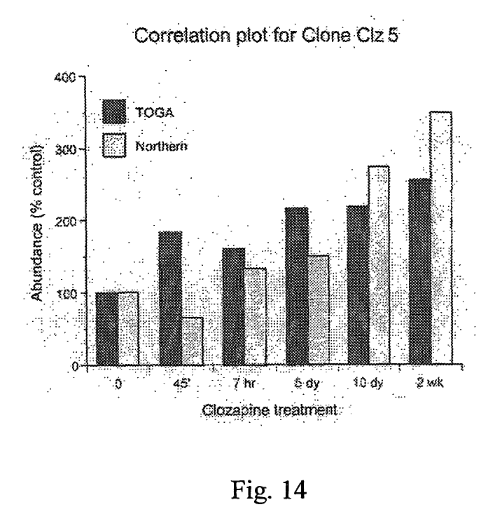

Northern Blot analyses were performed to determine the effect of clozapine on apoD expression in mouse striatum/nucleus accumbens. Shown in FIG. 13, Northern Blot analysis was performed using 2 μg poly A enriched mRNA extracted from the striatum/nucleus accumbens of control mice and clozapine-treated mice, as described in Example 3. A 160 bp insert of CLZ_{—}5 (25-100 ng) was labeled with [α-³²P]-d CTP by oligonucleotide labeling to specific activities of approximately 5×10⁸ cpm/μg, added to the prehybridization solution and incubated for 1 hour. Filters were washed to high stringency (0.2×SSC) (1×SSC: 0.015 M NaCl and 0.0015 M Na citrate) at 68° C. then exposed to Kodak X-AR film (Eastman Kodak, Rochester, N.Y.) for up to 1 week. Densitrometry analysis on Northern blots was performed by ImageQuant software. As can be seen in FIG. 13, a 900 bp mRNA was detected in control and clozapine-treated mice which corresponds with the apoD gene. The apoD mRNA expression is progressively up-regulated with clozapine treatment over the two-week time course. It is possible that clozapine may mediate its antipsychotic effect through the regulation of apoD. Alternatively, apoD may be co-regulated by clozapine, in parallel with the mechanism of the clozapine therapeutic effects, and can serve as an indicator of clozapine bioactive levels.

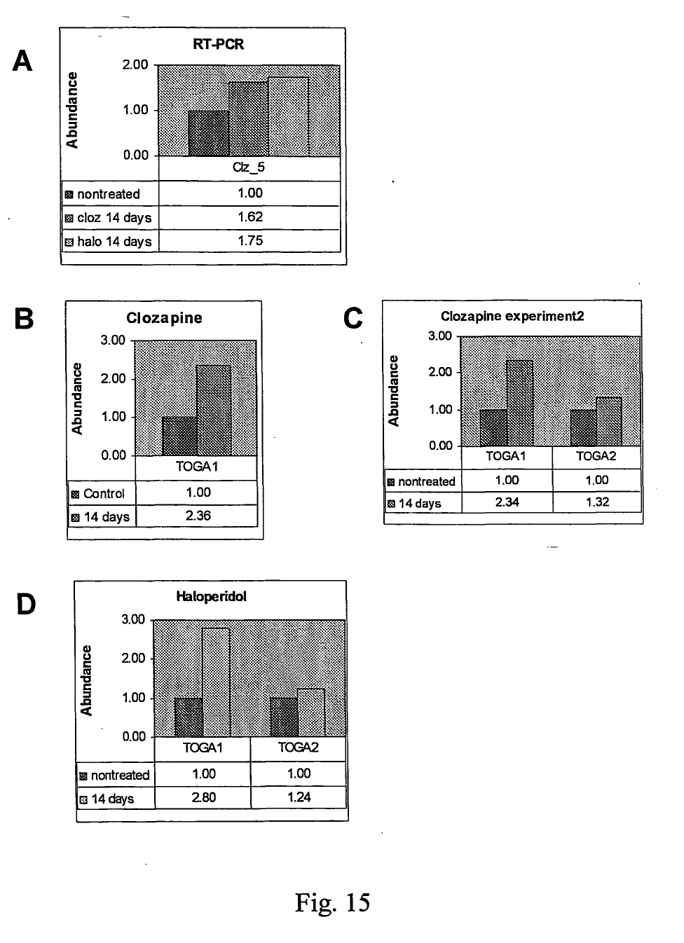

FIG. 14 is a graphical representation comparing the results of the clozapine treatment TOGA™ analysis of clone CLZ_{—}5 (CACC 201) shown in FIG. 12 and the clozapine treatment Northern Blot analysis of clone CLZ_{—}5 shown in FIG. 13. The Northern Blot was imaged using a phosphorimager to determine the amount of apoD mRNA in each clozapine-treated sample relative to the amount of mRNA in the control sample. As can be seen, the clozapine treatment TOGA™ analysis shows correlation with the clozapine treatment Northern Blot analysis.

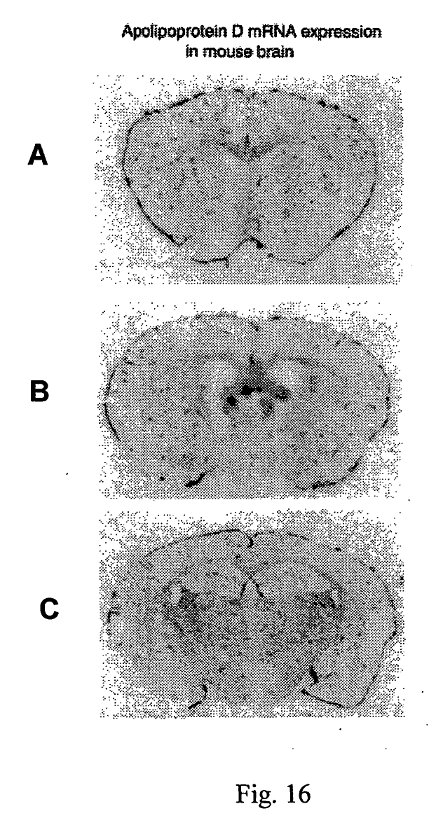

In addition to Northen Blot analysis, Real Time PCR was performed on cDNA from clozapine compared to haloperidol treated mice. FIG. 15A-D compares the results from Real Time PCR validation (A) to the TOGA™ result from three different experiments: the original clozapine experiment (B), a repeated clozapine experiment performed in duplicate (C), and a haloperidol experiment in duplicate (D). As the tables and graphs illustrate, Real Time PCR analysis demonstrates that the DST CLZ_{—}5 (SEQ ID NO:2) increased in expression in response to both clozapine (1.62-fold) and haloperidol (1.75-fold) treatment. Table 5 lists the 5′ and 3′ primers used in these Real-Time PCR studies. Thus, similar to CLZ_{—}40 (Example 2), a common expression pattern was observed after treatment with these two different neuroleptics. This common expression pattern suggests that CLZ_{—}5 is a strong candidate for an RNA encoding protein whose activity is involved in the benefit derived by patients from these two classes of neuroleptic drugs.

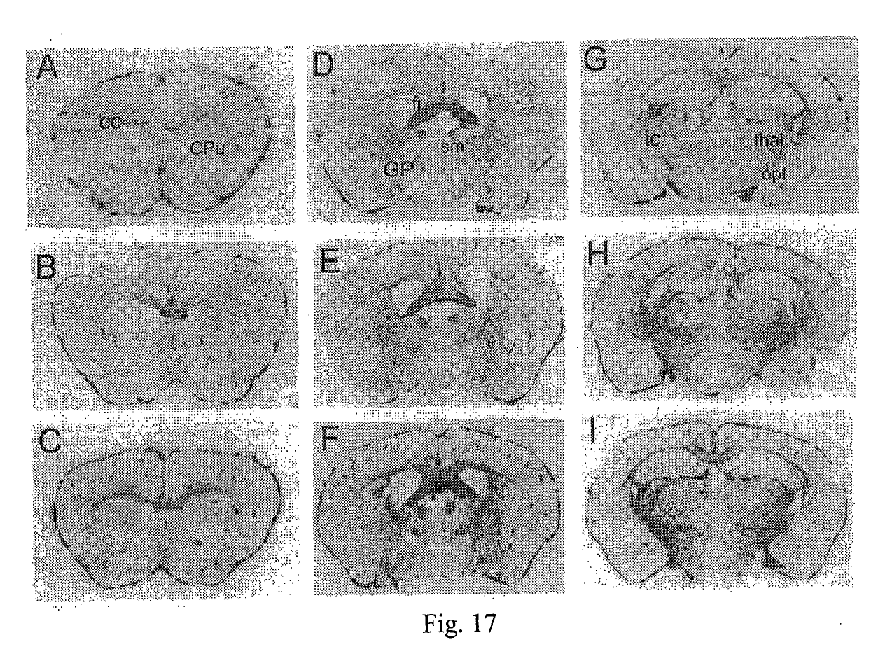

In order to determine the pattern of apoD expression in control and clozapine-treated mouse striatum/nucleus accumbens, in situ hybridization analyses were performed. FIG. 16A-C shows an in situ hybridization analysis, demonstrating the apoD expression in mouse brain. The in situ hybridization was performed on free-floating sections (25 μM thick) as described in Example 2. FIG. 16A shows CLZ_{—}5 (apoD) mRNA expression in mouse anterior brain, 16B shows apoD mRNA expression in midbrain and 16C shows apoD expression in posterior brain. In all brain sections apoD is expressed by astroglial cells, pial cells, perivascular fibroblasts and scattered neurons. This is consistent with previous studies examining the expression of apoD in mice, rabbits and humans (Yoshida et al., DNA and Cell Biology, 15, 873-882 (1996); Provost et al., J. Lipid Res., 32, 1959-1970 (1991); Navarro et al., Neurosci. Lett., 254, 17-20 (1998).

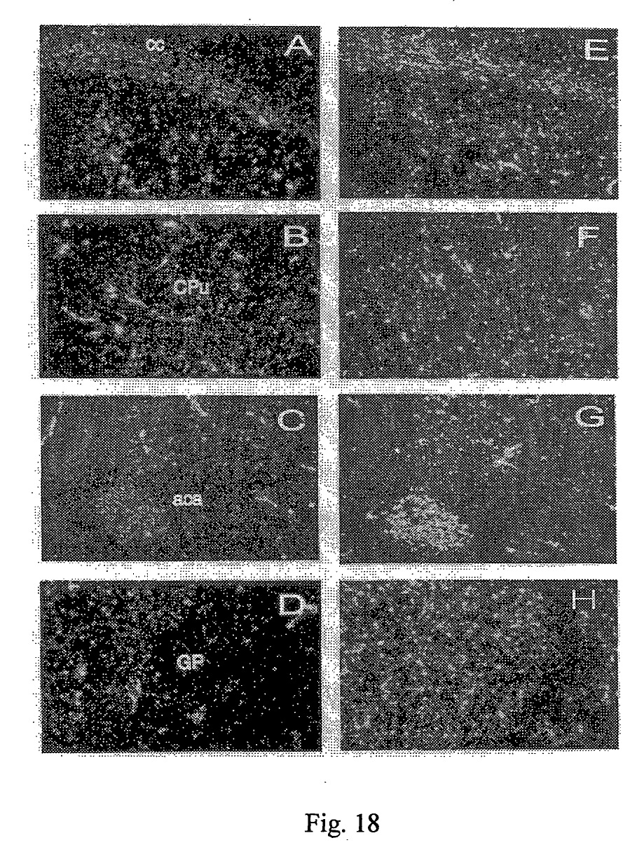

The Northern blot results (FIGS. 13 and 14) and Real Time PCR analysis (FIG. 15) indicated that apoD was induced by clozapine in the striatum of mouse brain. To investigate additional sites of apoD induction, in situ hybridization analysis was performed on brains from saline- and clozapine-treated mice. FIG. 17A-I presents an in situ hybridization analysis, showing clone CLZ_{—}5 (apoD) mRNA expression in mouse anterior (17A-C), mid (17D-F), and posterior (17G-I) brain following saline treatment (top row) or clozapine treatment (7.5 mg/kg) for 5 days (middle row) and 14 days (bottom row), using previously described methods. Animals were sacrificed by intracardial perfusion with 4% paraformaldehyde and the brains removed, post-fixed for 12 hours, cryoprotected with 30% sucrose and rapidly frozen at −70° C. At low magnification, increases in apoD mRNA were observed at both five days and two weeks of clozapine treatment in the striatum, cortex, globus pallidus (GP), and thalamus. Increases in apoD expression were also detected in white matter tracts, predominantly the corpus callosum (cc), anterior commissure, internal capsule (ic) and optic tract (opt). At high magnification, it was evident that the increased apoD hybridization signal in the striatum, globus pallidus, and thalamus of the drug-treated animals was primarily due to an increase in the number of cells expressing detectable apoD, although some cells with higher apoD expression were also observed.

Using a monoclonal antibody directed against full-length apoD, immunohistochemistry analyses were performed to evaluate changes in apoD protein expression in response to clozapine. Increase in protein expression correlated well with increases in mRNA expression (data not shown). Combined in situ hybridization and immunohistochemical studies demonstrated that increases in apoD levels were localized primarily to neurons and astrocytes of the striatum and oligodendrocytes in various white matter tracts throughout the brain.

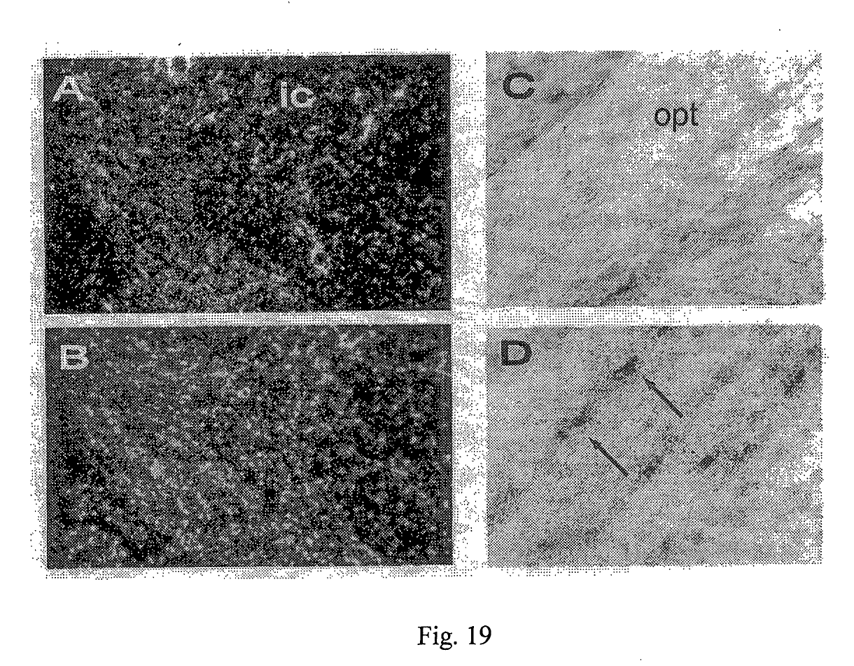

FIG. 18A-H shows a darkfield photomicrograph demonstrating upregulated apoD mRNA expression in various brain regions, including the corpus callosum (cc, FIG. 18A, E); caudate putamen (CPu, FIG. 18B, 18F); anterior commissure (aca, FIG. 18C, 18G); and globus pallidus (GP, FIG. 18D, 18H). In situ hybridizations were perfomed as described above, using an antisense ³⁵S-labeled apoD riboprobe on brains from control (FIG. 18A-D) and clozapine-treated (FIG. 18E-H) animals. The observed upregulation of apoD was due to an increase in the amount of apoD expressed per cell.

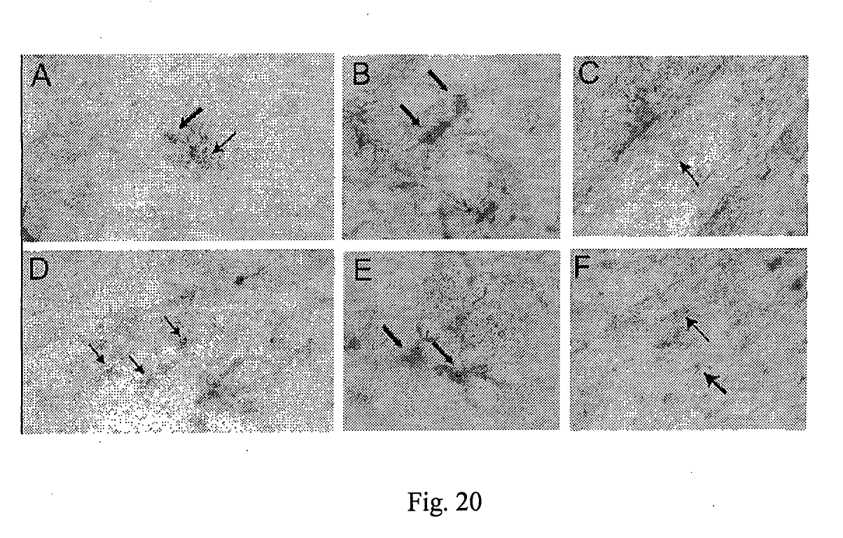

FIG. 19A, B shows a darkfield photomicrograph demonstrating upregulated apoD mRNA expression in the internal capsule (ic). FIG. 19C, D shows a brightfield view of the optic tract (opt) demonstrating upregulation of apoD expression in oligodendrocytes. In situ hybridizations were perfomed as described above, using an antisense ³⁵S-labeled apoD riboprobe on brains from control (19A, C) and clozapine-treated (19B, D) animals. As shown in FIG. 19D, the cells prominantly expressing apoD in the optic tract have a box-like morphology and are lined up in a serial array, presumably along axonal tracts. Such features are characteristic of oligodendrocytes, which synthesize the insulating myelin coating of nerve fibers. In situ hybridization experiments performed on brains from haloperidol-treated mice did not reveal substantial increases in apoD expression in gray or white matter regions (data not shown).

White matter tracts comprise nerve fiber bundles connecting different regions of the brain. The predominant cells in these regions are astrocytes and oligodendrocytes, both of which have been shown to express apoD (Boyles et al., J Lipid Res 31:2243-2256 (1990); Navarro et al., Neurosci Lett 254:17-20 (1995); Provost et al., J Lipid Res 32 (1991)). To determine which cell types are responsible for the increase in apoD signal, co-localization studies were performed using a 35S-labeled apoD riboprobe in combination with either an antibody specific for an astrocyte marker, glial fibrillary acidic protein (GFAP), or an antibody specific for an oligodendrocyte marker, 2′,3′-cyclic nucleotide 3′-phosphodiesterase (CNP) (Boehringer Mannheim, Germany). The immunoreaction was detected with Vectastain ABC™ kit (Vector Laboratory, Inc., Burlingame, Calif.) according to the manufacturer's instructions. Free floating brain sections were incubated with blocking solution (4% bovine serum albumin in 0.1% Triton X-100/PBS) for 2 hours at room temperature, followed by incubation with anti-GFAP or anti-CNP antiserum (dilution 1:500) in blocking solution for 16-20 hours at 4° C. Sections were then washed with 0.1% Triton X-100/PBS and incubated with secondary biotinylated antibody (1:200 dilution in blocking solution) for 2 hours at room temperature. The sections were then washed with 0.1% Triton X-100/PBS, incubated for 1 hour with ABC reagent (1:1 in blocking solution) and finally washed with 0.1% Triton X-100/PBS. Enzymatic development was performed in 0.05% diaminobenzene in PBS containing 0.003% hydrogen peroxide for 3-5 minutes.

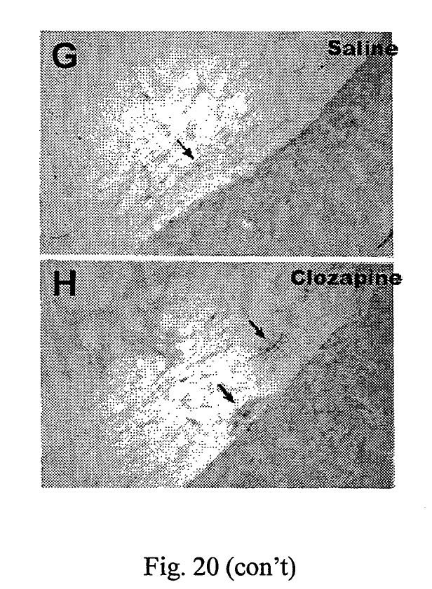

FIG. 20 shows sections of striatum and optic tract in control and clozapine-treated animals. Thick arrows indicate the co-localization of GFAP and apoD, while thin arrows indicate the expression of apoD alone. FIG. 20A, B shows that in untreated striatum, many GFAP-positive cells in both gray and white matter regions are positive for apoD. FIG. 20D, E shows that in brain from clozapine-treated animals, an increase in the amount of apoD was observed in a small subset of GFAP-positive cells in the striatum. Additionally, there was an increase in the number of non-GFAP-positive cells expressing apoD in the striatum, as well as the globus pallidus and thalamus, which are presumptively neurons, based on size and morphology. FIG. 20C, F shows GFAP and apoD co-localization in the optic tract in control (20C) and clozapine-treated (20F) animals. While some astrocytes express apoD mRNA, the cells responsible for the predominant apoD transcript upregulation did not label with GFAP and thus are likely oligodendrocytes. In other white matter regions, such as the corpus callosum, anterior commissure and internal capsule, the non-GFAP expressing cells that express apoD are likely to be oligodendrocytes as well, although expression in microglia cannot be ruled out. FIG. 20G, H shows apoD immunohistochemistry with an anti-human apoD primary antibody (Novocastra, Newcastle, UK) in the optic tract of control saline (20G) and clozapine-treated animals (20H).

Co-localization studies performed using anti-CNP antibody showed CNP immunoreactivity in white matter tracts throughout the CNS which correlated with areas of apoD mRNA hybridization signals, indicating the expression of apoD in oligodendrocytes. However, within the gray matter regions of the striatum, there was no co-localization consistent with the neuronal accumulation of apoD (data not shown).

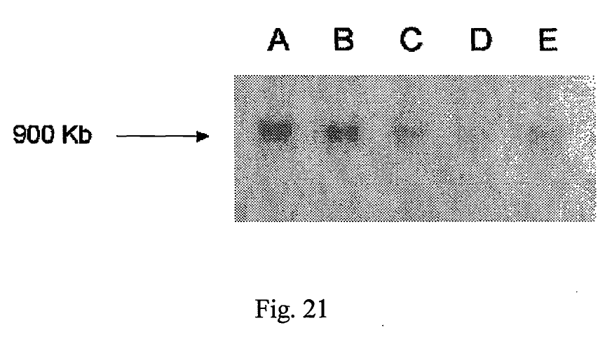

FIG. 21 shows a Northern Blot analysis of clone CLZ_{—}5 expression in cultured glial cells treated with clozapine (100 nM and 1 μM) for 1 day or 7 days. Glial cell cultures were produced from postnatal (day 2) rats. The cells were treated with different concentrations of clozapine for different lengths of time before mRNA extraction as follows: A=control (no clozapine), B=100 nM clozapine, 1 day, C=1 μM clozapine, 1 day, D=100 nM clozapine, 1 week, E=1 [M clozapine, 1 week. 20 μg of total cytoplasmic RNA from glial cell cultures were electrrophoresed on a 1.5% agarose gel containing formaldehyde, blotted, and probed as previously described. Interestingly, apoD mRNA levels were down-regulated in mixed glial cell cultures treated with clozapine (both 100 nM and 1 μM) for 1 week, suggesting that perhaps neurons and glia display different mechanisms for apoD regulation.

TOGA™ methodology, Northern blot analyses, Real Time PCR, and in situ hybridization studies have demonstrated an increase in apoD mRNA expression in both white and gray matter regions of mouse brain in response to chronic clozapine administration. Co-localization studies, combining in situ hybridization and imunohistochemistry methods have revealed that apoD mRNA levels are increased in both neurons and glial cells with clozapine administration. The evidence indicates that the glial cells responsible for the most dramatic increases in apoD expression are primarily oligodendrocytes, but a subset of astrocytes also have increased apoD expression after clozapine treatment. In addition, Real-Time PCR analysis suggested that apoD expression was also affected by haloperidol treatment.

The observation that, of thousands of proteins expressed by the striatum, a novel oxysterol binding protein (CLZ_{—}43, Example 2) and apoD are among the few modulated by neuroleptic drugs strengthens the hypothesis that schizophrenia is a disease of brain sterol homeostasis, and thus may have etiologies as diverse as atherosclerosis. The brain has by far more cholesterol and 24S-hydroxysterol than any organ other than the adrenal glands, and the special importance of the membrane activities of neurons and their myelinating cells are self-evident. The lipid bilayer of the membrane is made up of glycerolphopholipids and cholesterol, and variations in composition and hydrocarbon chain saturation state determine membrane order and fluidity. These properties affect the binding of extrinsic membrane proteins and, thus, second messenger signaling. As we have shown previously, a large percentage of the mRNAs highly enriched in the striatum encode proteins that regulate second messenger signaling along the inner membrane. Thus, a panneural or panorganismic disruption in lipid metabolism might manifest first as a striatal disease.

In addition to the mouse studies described above which show that apoD is regulated by chronic antipsychotic drug administration, studies using schizophrenic and bipolar human subjects showed that apoD expression is increased in the prefrontal cortex of such patients. The combined results suggest that apoD is a marker for neuropathology associated with psychiatric disorders and therefore can be used to target abnormalities in specific anatomical brain regions. The human studies are described in detail-in Example 5, below. In addition,′ Example 6 describes studies investigating a potential role for apoD in the neuropathology of Alzheimer's disease. As described in Example 6, apoD mRNA expression was measured in transgenic mice expressing mutated human amyloid precursor protein under control of platelet-derived growth factor promoter (PDAPP mice), and the findings suggest that, although increases in apoD expression are a normal feature of brain aging, super-increases may represent a glial cell compensatory response to beta-amyloid deposition in Alzheimer's disease.

ApoD was initially identified as a constituent of plasma high-density lipoproteins (HDLs), which also contain phospholipids, cholesterol and fatty acids (McConathy et al., Fed. Eur. Biochem. Soc. Lett., 37: 178 (1973)). In the blood, apoD is thought to play a role in reverse cholesterol transport, the removal of excess cholesterol from tissues to the liver for catabolism (Oram et al., J. Lipid. Res., 37: (1996)). In addition to abundant expression in human serum, apoD is major protein component in cyst fluid from women with human breast cystic disease (Balbin et al., Biochiem. J, 271: 803 (1990)) and also is widely expressed in numerous tissues, including liver, kidney, intestine, spleen and brain (Drayna et al., J. Biol. Chem., 261: (1986)). In the CNS of humans, as in other species (Provost et al., J. Lipid Res., 32: (1991); Seguin et al., Mol. Brain Res., 30: 242 (1995); Smith et al., J. Lipid Res., 31: 995 (1990)), apoD is expressed primarily in glial cells, pial cells, perivascular cells, and some neuronal populations (Navarro et al., Neurosci. Lett., 254: 17 (1995); Kalman et al., Neurol. Res., 22: 330 (2000)). The physiological role for apoD within the CNS is not known, however, it has been shown to bind several hydrophobic ligands, including sterols and steroid hormones (Dilley et al., Breast Canc. Res. Treat., 16: 253 (1990); Lea, O. A., Steroids, 52: 337 (1988)) suggesting a role in extracellular lipid transport in the brain. ApoD has also been shown to bind arachidonic acid (Morais-Cabral et al., FEBS Lett., 366: 53 (1995)) implicating it in functions associated with cell membrane remodeling and prostaglandin synthesis. In the regenerating sciatic nerve, a process that involves massive membrane degradation and lipid release, apoD concentrations are increased 500-fold (Boyles et al., J. Biol. Chem., 265: 17805 (1990)). Recent reports have also demonstrated an increase in apoD expression in rat brain after experimental and chemical lesioning of the entorhinal cortex and hippocampus, respectively (Ong et al., Neurosci., 79:359 (1997); Terisse et al., Mol. Brain Res.,70: 26 (1999)). Additionally, in humans, apoD accumulates in the cerebrospinal fluid and hippocampi of patients with Alzheimer's, and other neurological diseases (Terisse et al., J. Neurochem., 71: 1643 (1998)). Hence, apoD may be functioning during pathological situations or its expression may represent an effort to compensate for neuropathology associated with such insults.

The pattern of apoD expression in the brain suggests that apoD may play an important role in psychotic disease. It is widely believed that imbalances in basal ganglia circuitry contribute to psychotic behaviors and that blockade of specific receptors in these regions is responsible for neuroleptic action. The neuronal increases in apoD mRNA expression observed in neurons of the striatum and globus pallidus are consistent with this hypothesis.

In addition, the apoD induction observed in the internal capsule is of particular interest. The internal capsule consists of massive nerve fibers connecting the thalamus to the cortex and is an area of convergence for the fiber tracts running transversely through the striatum. The thalamus is a relay station for virtually all information passing to the cortex and coordinated cortico-thalamic activity is essential for normal consciousness. Recent theories have associated psychotic behavior with disruptions in cortico-thalamic oscillations. An upregulation of apoD expression in the internal capsule may play a role in restoring the proper balance of neuronal communication.

In addition, abnormal lipid neurochemistry resulting from abnormal lipid transport or metabolism has been associated with psychotic disease, such as schizophrenia (Walker et al., Br. J. Psych., 174, 101-104 (1999)). Relating impaired cholesterol metabolism with psychotic disease, a number of reports have described psychoses as an initial manifestation of Neimann-Pick Disease, type C (Campo, et al., Develop. Med. and Child Neurol., 40, 126-129 (1998); Shulman, et al., Neurology, 45, 1739-1743 (1995); Turpin, et al., Dev. Neurosci., 13, 304-306 (1991)), which is an autosomal recessive disease associated with abnormal cholesterol metabolism (Yoshida et al., DNA and Cell Biology, 15, 873-882 (1996)). Further reports have suggested that myelin dysfunction may cause mental illness. Given that the majority of cholesterol in the brain is incorporated into myelin, abnormal cholesterol metabolism may result in myelin dysfunction. Myelin acts as an insulator along nerve axons allowing for the rapid propagation of action potentials along nerve fibers. Molecular abnormalities of myelin may result in the dysregulated neural connectivity that has been hypothesized to be causative in mental illnesses (Weickert, et al., Schizophrenia Bull., 24, 303-316 (1998)).

Several lines of evidence suggest a role for apoD as a vehicle for extracellular lipid transport and lipid movement, particularly cholesterol, in the nervous system. ApoD is a constituent of plasma high-density lipoproteins (HDLs), which also contain phospholipids, cholesterol and fatty acids. While not much is known about HDL compared to the other plasma lipoproteins, LDL and VLDL, it is widely believed that HDLs protect against cardiovascular disease by removing excess cholesterol from cells of arterial walls. This removal involves the direct interaction of HDL-lipoproteins with plasma membrane domains and subsequent transport to the liver for catabolism (Oram, et al., J. Lipid Res., 37, 2473-2491 (1996)). Additionally, apoD is synthesized and secreted by cultured astrocytes, which secretion has been shown to increase in the presence of cholesterol derivatives (Patel, et al., Neuroreport 6, 653-657 (1995)). Further, it has also been demonstrated that apoD levels are increased in Niemann Pick Disease, type C, which is associated with elevated levels of cholesterol. These studies provide evidence of a functionally significant role for apoD in cholesterol transport in the CNS.

In addition to the studies correlating cholesterol levels and psychotic behavior, other studies have found a correlation between cholesterol levels and treatment with neuroleptics. For example, reports dating back to 1960 have demonstrated an increase in the serum cholesterol of patients treated with conventional neuroleptics, such as chlorpromazine and haloperidol (Spivak et al., Clin. Neuropharm., 22, 98-101 (1999). Fleischhacker et al., Pharmacopsychiatry, 19, 111-114 (1986); Clark et al., Clin. Pharm. and Therapeutics, 11, 883-889 (1970)). However similar increases are not observed with the newer, atypical antipsychotics, such as fluperlapine and clozapine (Spivak et al., Clin. Neuropharm., 22, 98-101 (1999). Fleischhacker et al., Pharmacopsychiatry, 19, 111-114 (1986); Boston, et al., Biol. Psych., 40, 542-543 (1996)). Interestingly, the present results reveal that clozapine and haloperidol have a differential effect on apoD expression, which may account for the observed differences in cholesterol regulation. While the mechanism for these cholesterol changes is not known, the present data suggest that neuroleptic-induced changes in apoD expression combined with the ability of apoD to bind cholesterol may provide an explanation for the neuroleptic-induced changes in cholesterol levels.

In addition to studies relating to cholesterol movement, reports have focused on the link between disrupted phospholipid and fatty acid metabolism and psychiatric disorders (for a review see Horrobin, et al., Prostaglandins, Leukotrienes and Essential Fatty Acids, 60, 141-167 (1999)). For example, numerous studies have reported differences in levels of total membrane phospholipid content, fatty acid levels, cholesterol levels and cholesteryl esters in fibroblasts and/or frontal cortex of schizophrenics (Keshavan et al., J Psychiatry Res., 49, 89-95 (1993); Mahadik et al., Schizophrenia Res. 13, 239-247 (1994); Sengupta et al., Biochem. Med., 25, 267-275 (1981); Stevens, Schizoplr. Bull., 6, 60-61 (1972)). Membrane phospholipids act as precursors in numerous signaling systems (e.g., inositol phosphates, arachidonic acid, platelet activation factors, and eicosaniods) and comprise the membrane environment for neurotransmitter-mediated signal transduction. Thus, altered membrane phospholipid metabolism could have significant consequences for neuronal communication, resulting in behavioral abnormalities.

Alterations in plasma membrane structure and function may result from the altered content and distribution of membrane lipids and fatty acids, such as arachidonic acid. Arachidonic acid is released by the action of numerous phospholipase enzymes, primarily phospholipase A2, and is a substrate for prostglandins and leukotriene synthesis. While the molecular mechanisms underlying abnormalities in the complex system of phospolipid biochemistry are not known, several groups have demonstrated an increase in phospholipase A2 activity in the plasma and brains of schizophrenic patients (Gattaz et al., Biol. Psychiatry., 22, 421-426 (1987); Ross et al., Arch. Gen. Psychiatry., 54, 487-494 (1997); Ross et al., Brain Research, 821, 407413 (1999)). In addition, plasma phospholipase A2 levels have been shown to be decreased after neuroleptic therapy (Gattaz et al., Biol. Psychiatry, 22, 421-426 (1987)). Other molecular candidates implicated in psychotic disease include phospholipase C enzymes, diacyl glycerol kinases, and inositol phosphates (Horrobin et al., Prostaglandins, Leukotrienes and Essential Fatty Acids, 60, 141-167 (1999)).

Interestingly, in addition to binding cholesterol, apoD has been shown to specifically bind arachidonic acid. ApoD is an atypical apolipoprotein in that it does not share sequence homology with other apolipoproteins (Weech et al., Prog. Lipid Res., 30, 259-266 (1991)) but, rather, is a member of the lipocalin superfamily of proteins, which function in the transport of small hydrophobic molecules, including sterols, steroid hormones, and arachidonic acid (Balbin et al., Biochem. J., 271, 803-807 (1990); Dilley et al., Breast Cancer Res. Treat., 16, 253-260 (1990); Lea, Steroids, 52, 337-338 (1988); Boyles et al., J. Lipid Res., 31, 2243-2256 (1990)). As a lipid binding protein, apoD can affect fatty acid composition, cholesterol levels and membrane phospholipids, all of which will affect plasma membrane composition and structure. Also, since apoD specifically binds cholesterol, arachidonic acid and other lipids, alterations in the levels of apoD can affect lipid metabolism and signal transduction by affecting substrate availability for these pathways.

Further implicating the role of apoD in psychosis is the observation that apoD may have a chromosomal linkage with schizophrenia. The chromosomal location of apoD is 3q26. Genetic studies have implicated a potential association between schizophrenia and chromosome 3q, however the linkage is relatively inconsistent (reviewed by Maier, et al., Curr. Opin. Psych., 11, 19-25 (1998)).

It is possible that a serotonin sub-type such as 5HT₂ₐ and ⁵HT_{2c} may provide a pharmacological mechanism for clozapine's effect on apoD expression. Preliminary results demonstrate that treatment with ketanserin and mesulergine, 5HT_{2a/2c} and 5HT_{2c}-selective antagonists respectively, results in an apparent upregulation of apoD mRNA expression in mouse brain. It is known that the striatum expresses a number of 5HT receptor subtypes, including the 5HT_{2c}, which subtype may mediate clozapine's effect on apoD expression. In contrast, cultured glial cells or astrocytes do not appear to express 5HT_{2c} receptors. Thus the downregulation observed in these cells may reflect actions at a different 5HT subtype, such as 5HT₂ₐ, or a different receptor. Additionally, in hypertension studies, ketanserin has been associated with a decrease in total cholesterol levels and an upregulation of another apolipoprotein, apo A1 (Loschiavo, et al., Int. J Clin. Pharmacol. Ther. Toxicol., 28, 455-457 (1990)). The similar effects observed by both ketanserin and clozapine suggest that they may be working through the same receptor subtype(s).

The finding that apoD mRNA levels are increased by clozapine links apolipoproteins and the mechanism of action of neuroleptic drugs. The proposed role of apoD in CNS lipid transport, combined with the recent evidence that schizophrenia and other neuropsychiatric illnesses are accompanied by abnormalities in lipid metabolism, suggest that apoD could play an important role in the action of clozapine.

### EXAMPLE 5

### Characterization of ApoD in Schizophrenic and Bipolar Human Subjects

The mouse studies described above (Example 4) show that apoD is regulated by chronic antipsychotic drug administration and the pattern of apoD expression in the brain suggests that apoD may play an important role in psychotic disease. This example demonstrates that apoD expression is increased in the dorsolateral prefrontal cortex region (BA9) and caudate of the brains of schizophrenic and bipolar human subjects compared with control human subjects. The combined results suggest that apoD is a marker for neuropathology associated with psychiatric disorders and therefore can be used to target abnormalities in specific anatomical brain regions.

Numerous studies have reported dysfunctions of a variety of neurotransmitter receptor systems in schizophrenic patients (Dean, B., Australian and New Zealand J Psych., 34: 560 (2000); Harrison, P. J., Brain, 122: 593 (1999)). Although the previously described rodent studies. (see Example 4) were performed on mice that exhibit normal dopaminergic, serotonergic and glutamatergic functions, the studies implicate apoD in pathways associated antipsychotic drug action and indicate that apoD may be a reporter for clozapine function. Increased apoD expression may be a result or consequence of other neurobiological defects governing the presentation of psychiatric disturbances. Therefore, apoD levels in human brains of control, schizophrenic and bipolar patients were measured.

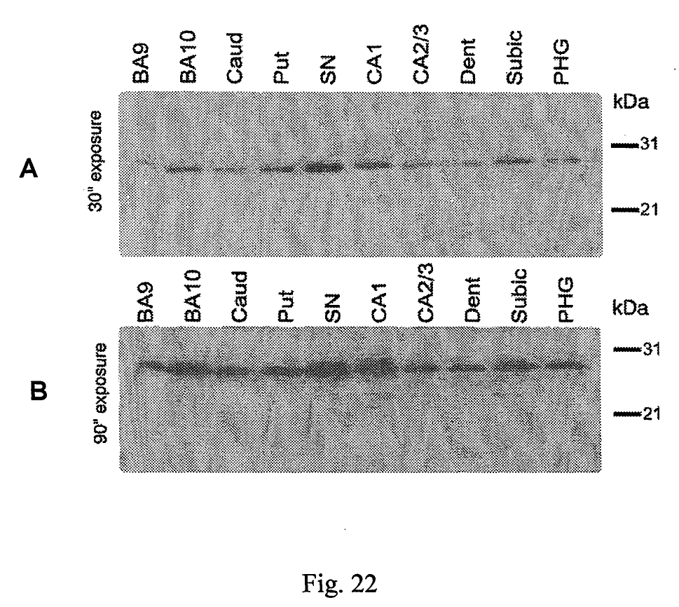

For the data presented in FIGS. 22-24, the following methods described below were used. All methods were in accordance with the North-Western Health Care Human Ethics Committee of the Victorian Institute of Forensic Medicine. Tissue samples were obtained from the left brain hemisphere of 18 subjects with a provisional diagnosis of schizophrenia and 8 subjects with bipolar disorder (see Tables 6 & 7). Tissue was also collected from 19 subjects (controls) with no known history of psychiatric illness such that the sex distribution and mean age of the control and schizophrenic groups were similar (Table 6). The control group used in the bipolar study was a subset of the control subjects used in the schizophrenic study. Control subjects were chosen in attempts to match gender and age distribution, however the mean age of the bipolar group was significantly greater (Table 7). These control subjects were re-analyzed in separate experiments with the bipolar subjects. In all cases, the cadavers were refrigerated within 5 hours of being found and the tissue was rapidly frozen to −70° C. within 30 minutes of autopsy and stored until used. The mean post-mortem interval for the tissue from each group was not significantly different (Tables 6 & 7). To attempt to address any effects of agonal state on the brain samples, the pH of the brain tissue was measured as described previously (Kingsbury et al., Mol. Brain Res., 28:311-318 (1995)), and was similar between groups. The provisional diagnosis of schizophrenia or bipolar was confirmed by a senior psychiatrist after an extensive case history review (Hill et al., Am. J Psychiatiy, 153:533-537 (1996)). In this study, all diagnoses were confirmed using DSM-IV criteria (Association, A.P. Diagnostic and statistical manual of mental disorders, Fourth Ed., American Psychiatric Association, Washington, D.C. (1994))), and was not significantly different between control and diseased groups. Duration of illness (DOI) was calculated at the time from first hospital admission to death. In addition, information on the type and amount of antipsychotic drugs prescribed close to death was obtained from the case history. All schizophrenic patients, and six of the eight bipolar subjects, had a history of treatment with typical neuroleptic drugs, except two who were reported to have been treated with clozapine and another that had been neuroleptic-free for over 1 year (Table 6).

Membrane homogenates were prepared from various brain regions (prefrontal cortex, occipital cortex, substantia nigra, cerebellum, hippocampal formation, and caudate) of control, schizophrenic or bipolar subjects by homogenization in Tris buffer (20 mM Tris-HCl, 0.2 mM EGTA, 0.1 mM EDTA, pH 7.4) including 3× “complete” protein inhibitor tablets (Boehringer Mannheim). Aliquots of the membrane homgenates (50 μg total protein/lane) were subjected to sodium dodecyl sulfate-polyacrylamide gel electrophoresis (SDS-PAGE) using a 12% acrylamide gel. The gels were transferred to nitrocellulose membranes, blocked with 5% milk in T-TBS (Tris-buffered saline/0.11% Tween-20, pH 7.5) and then probed with a monoclonal antibody directed against human apoD) (1:500 dilution) (Novacastra). Enhanced chemiluminescence (ECL, Amersham, Arlington Heights, Ill.) was used to detect immunoreactivity and blots were visualized by exposure to autoradiography film.

FIG. 22 shows Western blot analyses of various brain sections in control human subjects. Western blots containing 50 μg total protein per lane were probed with a monoclonal antibody directed against apoD. Enhanced chemiluminescence (ECL) was used to detect immunoreactivity and blots were visualized by exposure′ to autoradiography film. As shown, Western blot analysis using an antibody specific to apoD revealed widespread distribution of apoD protein in various human brain regions, including prefrontal cortex (BA9 and BA10), components of the hippocampal formation (CA1, CA3, dentate gyrus, subiculum, parahippocampal gyrus) and basal ganglia (caudate and substantia nigra) (see FIG. 22). ApoD immunoreactivity was detected in all brain regions tested, with the highest level of expression observed in the substantia nigra (SN). A major band of 29 kDa was observed in all regions examined, however, an additional band of approximately 22 kDa was observed in other brain regions, primarily Brodman's Area 10, substantia nigra, CA1 and subiculum. Molecular variation of apoD has been reported previously in human plasma and regenerating rat sciatic nerve and likely reflects different glycosylation states of the protein (McConathy et al., Fedn Eur Biochem Socs Lett, 3 7:178 (1973); Boyles et al., J Biol Chem, 265: 17805 (1990); Kamboh, et al., Am J Hum Genet, 45: 147 (1989)).

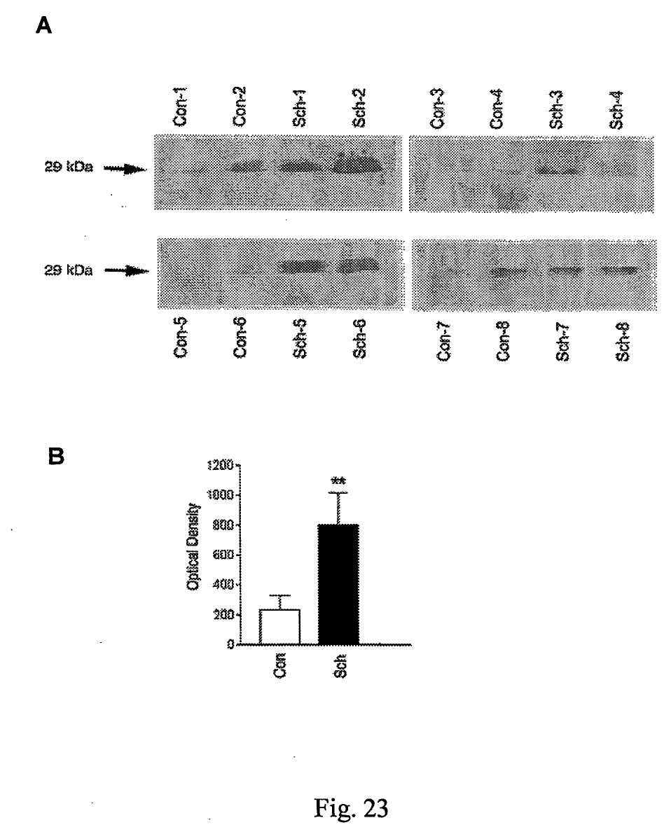

FIG. 23A-B are Western blot analyses showing apoD expression levels in the dorsolateral prefrontal cortex, Brodman's Area 9 (BA9), of eight schizophrenic subjects (Sch-1 to Sch-8) and eight age- and sex-matched control subjects (Con-1 to Con-8). Brodman's Area 9 is a region previously implicated in the pathophysiology of schizophrenia (for review, see Goldman-Rakic et al., Schiz. Bull., 23: 437 (1997)). Western blots containing 50kg total protein per lane were probed with a monoclonal antibody directed against apoD. Enhanced chemiluminescence (ECL) was used to detect immunoreactivity and blots were visualized by exposure to autoradiography film. As shown in FIG. 23B, Western blots quantified by densitometric analysis revealed significantly elevated levels of apoD in the BA9 regions of schizophrenic patients (802.5±217 O.D. units; P=0.0232) relative to age- and sex-matched control subjects (207±85.0 O.D. units) (see FIG. 23).

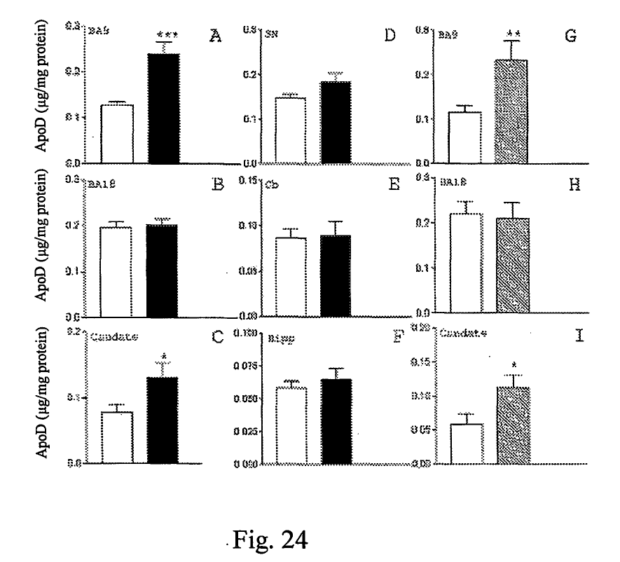

To more accurately quantify the levels of apoD in these subjects, ELISA assay was performed to measure apoD levels in BA9 regions, as well as caudate and occipital cortex (BA18) taken from the same and additional subjects (n=19 for each control and schizophrenic). Two monoclonal antibodies to apoD from Signet Laboratories, Inc. (Dedham, Mass., USA) were used in a sandwich assay. Microtitre high capacity binding plates (Costar) were coated with 50 μl of a 4.7 μg/ml of apoD antibody for 1-2 hrs at room temperature. The wells were washed 4× with T-TBS, blocked with 5% bovine serum albumin in T-TBS for 1 hour at room temperature and then washed again 4× with T-TBS. An aliquot (50 μl) of the various tissue homogenates (50 μg total protein) was added and incubated for 1 hr at room temperature. The wells were washed 4× with T-TBS, and then 50 μl of a second, HRP-conjugated, apoD antibody was added to each well and incubated 1 hour at room temperature. After extensive washing with T-TBS, 50 μl of TMB substrate system (Sigma Chemical Co.) was added to allow color formation. The reaction was quenched with 0.2 N HCl (50 μl) and absorbance was read at 450 nM. Purified apoD (kindly provided by Dr. D.A. Haagensen, Scramento, Calif.) was used as a standard in all assays. The ELISA data were subjected to 2-way ANOVA to discern significant differences among brain regions from the same cohort of subjects, and then student's t test (two-tailed) was used to determine exact P values. All statistical analyses, student's t test, 2-way ANOVA and linear regression analysis, were carried out using Prism computer software. The results are shown in FIG. 24A-I and summarized in Table 8.

As shown in FIG. 24A, a significant increase (p=0.0002) was detected in the dorsolateral prefrontal cortex (DLPFC, BA9) from schizophrenic patients (0.244±0.027 μg/mg protein; n=20) (p=0.0002) versus controls (0.127±0.008 μg/mg protein; n=19). FIG. 24C shows ELISA assay measurements performed in the caudate from the same schizophrenic patients and control subjects used to determine apod levels in the BA9 regions. A significant increase (p=0.045) was detected in the caudate from schizophrenic patients (0.132±0.021 μg/mg protein) relative to controls (0.078±0.011 μg/mg protein) (FIG. 24C). ApoD concentrations were also measured in the occipital cortex (OC, Brodmann's Area 18, BA18), substantia nigra (SN), cerebellum (Cb), and hippocampus (Hipp) of these same subjects. No difference was found in the apoD levels between control and schizophrenic subjects in these brain regions (FIG. 24D-F).

To test for disease specificity, apoD expression levels were also measured in prefrontal and occipital cortices and caudate from patients diagnosed with bipolar disease and from a subset of the control subjects (FIG. 24G-I, Table 8). As shown in FIG. 24G, increased apoD concentrations were detected in the BA9 region of the bipolar patients, similar in magnitude to that observed in schizophrenic patients (bipolar, 0.233±0.043 μg/mg protein; n=8; p=0.0424 versus control, 0.115±0.015 μg/mg protein; n=8). A significant increase in apoD expression was also observed in the caudate of bipolar (0.7112±0.018 μg/mg protein; n=8; p=0.0218) versus control subjects (0.059±0.015 μg/mg protein; n=8) (FIG. 24I), while the apoD levels in the occipital cortex were not different between control and bipolar subjects (FIG. 24H). Hence, the increases in apoD expression are not restricted to patients with schizophrenia. Additional studies have been performed to determine the spectrum of elevated apoD expression in the CNS of schizophrenic subjects. Six additional brain regions have been measured and summarized with the results from FIG. 24A-H in Table 8. In summary, out of the twelve regions studied, six exhibited significantly elevated expression of apoD, including, dorso-laterial prefrontal cortex, lateral prefrontal cortex, orbito-frontal cortex, caudate, thalamus and amygdala.

These results show that apoD expression is significantly increased (1.9- to 3.9-fold) in dorsolateral prefrontal cortex of schizophrenic and bipolar patients, which is a brain region previously implicated in the pathophysiology of schizophrenia. In contrast, apoD expression is not increased in the occipital cortex, a region with no association to schizophrenia. Numerous experimental and clinical studies have provided evidence of pathophysiological changes in the prefrontal cortex of patients with schizophrenia. Studies using neuroimaging techniques have demonstrated decreased blood flow activation and metabolism in prefrontal cortex of schizophrenic patients, especially during behavioral tasks (Franzen et al., J Neurol Neurosurg Psychiatry, 38: 1027-1032 (1975); Weinberger et al., Arch Gen Psychiatry, 43: 114-143 (1986); Liddle et al., Br J Psychiatry, 158: 340-345 (1991); Ragland et al., Neuropsychology, 12: 399413 (1998); Carter et al., Am J Psychiatry, 155: 1285-1287 (1998)). Neuropsychological and neurophysiological observations of schizophrenic patients have also revealed impairments in cognitive tasks and working memory skills, behavioral processes that require intact prefrontal functioning (Weinberger et al., Arch Gen Psychiatry, 43:114-143 (1986); Liddle et al., Br J Psychiatry, 158: 340-345 (1991)).

There were no significant differences in apoD expression in the occipital cortex (BA18), substantia nigra, cerebellum or hippocampus, indicating regional specificity for apoD expression induction. The increases in apoD levels observed in the DLPFC and caudate of bipolar subjects, indicate that increased apoD accumulation is not specific to schizophrenia. However, components of bipolar disorder have also been associated with abnormal functioning of the prefrontal cortex (Blumberg et al., Am J Pyschiatry, 156: 1986-1988 (1999); Knable, M. B., Schizophr Res, 39: 149-152 (1999); Drevets et al., Mol Psychiatry, 3: 220-226 (1998)). The increases observed in the caudate are also consistent with studies implicating basal ganglia structures in the pathophysiology of psychiatric disorders.

Previous studies have demonstrated an increase in apoD immunoreactivity in the CSF and hippocampi of Alzheimer's patients, and in the CSF of patients with other neurological diseases, including cerebrovascular disease, motorneuron diseases and meningoencephalitis (Terrisse et al., J Neurochem. 71: 1643 (1998)). These studies and present studies discussed herein suggest that apoD may be a marker for neuropathology. Additional studies in Alzheimer's subjects did not detect increases in cortical regions of the brain (Kalman et al., Neurol Res., 22: 330 (2000)) in contrast to our studies in the brains of schizophrenia and bipolar patients. This suggests that apoD is regionally altered in diseased brain and that expression is induced in only in regions central to pathology of a given neurological disorder.

As un-medicated schizophrenic patients are difficult to acquire, all of the schizophrenic subjects in this study had been treated with typical neuroleptic drugs (haloperidol, fluphenazine, thioridazine or chlorpromazine) with the exception of two, who were treated with the atypical drug, clozapine, and another who was neuroleptic-free for 1 year prior to death. Six of eight of the bipolar patients were also reported to have been treated with typical neuroleptic drugs prior to death. Thus, it is possible that the observed increases in apoD could be a consequence of long-term neuroleptic drug treatment. However, no correlation was observed between apoD levels and antipsychotic drug dose (chlorpromazine equivalents) in these subjects (r²=0.0111), suggesting that the effect of apoD is not a consequence of neuroleptic drug treatment. In addition, the fact that apoD levels are also increased in brains of patients who are presumably not medicated with neuroleptic drugs (i.e. patients with Alzheimer's and other neurological diseases) also supports the hypothesis that increased apoD levels are not resulting from drug treatment, but rather are an indicator for, or consequence of, neuropathology.

In addition, since apoD expression is elevated under apparently diverse conditions, it is possible that apoD expression represents a non-specific response to stress or pathological insult. However, given the distinct sites of apoD upregulation observed after CNS insult in the rodent studies and the regional specificity of apoD induction observed in human disease and mouse models (Niemann-Pick), rather, it is possible that apoD is a region-specific marker for active pathological processes. Our findings that clozapine induced apoD accumulation in rodent brains had suggested the simple hypothesis that increases apoD may be beneficial to patients with neuropsychiatric disorders. The present findings suggest that apoD accumulation might be a natural response to regional neuropathology, and that one reason clozapine is an effective antipsychotic drug is via its ability to augment increases in apoD already present in the brain.

As discussed earlier in Example 4, ApoD has been shown to specifically bind the fatty acid, arachidonic acid, which together with docosahexaenoic acid, make up >90% of the polyunsaturated fatty acid content in the CNS (O'Brien, et al., J Lipid Res., 6: 537-544 (1965)), and is also thought to play a role in the transport of cholesterol, which makes up 25% of gray and white matter (Snipes et al., Subcellular Biochem, (Ed Bittman, R. (Plenum Press, New York 1997)). Both of these are major components of the lipid bilayer of cellular membranes. Variation in composition and hydrocarbon chain saturation state determine membrane order and fluidity, and these properties affect the binding and function of extrinsic membrane proteins and second messenger signaling. Hence, changes in the levels of apoD can potentially affect membrane phospholipid composition, by increasing or decreasing transport and uptake of these membrane constituents. Phospholipids play a critical role in almost every function of the cell membrane and its metabolic products are crucial for cellular functions and cell-to-cell communication.

In the prefrontal cortex, a site of increased apoD expression, numerous reports have demonstrated increases and/or decreases in neurotransmitter receptors, ion channels and membrane-bound proteins in subjects with schizophrenia and other psychiatric disorders. Alterations in dopamine, serotonin, glutamate and GABA neurotransmitter receptors have been demonstrated in the prefrontal cortex of schizophrenic subjects (for review see Dean, B., Australian and New Zealand J Psychiatry, 34: 560-569 (2000); Harrison, P. J., Brain, 122: 593-624 (1999)). In addition, abnormalities in molecules responsible for the synthesis, release and uptake of neurotransmitters have been reported. For example, changes in the expression and distribution of synthetic enzymes for neurotransmitters, such as nitric oxide and GABA, have been observed in frontal cortex regions of schizophrenic subjects (Akbarian et al., Arch Gen Psychiatry, 50: 169-177 (1993); Akbarian et al., Arch Gen Psychiatry, 52: 258-266 (1995)), and dysfunction in neurotransmitter uptake systems, such as the 5HT transporter and GABA and glutamate uptake sites have been reported in similar regions (Dean, B., Australian and New Zealand J Psychiatry, 34: 560-569 (2000); Harrison, P. J., Brain, 122: 593-624 (1999)).

Alterations in membrane phospholipids and consequential effects on neural cell membranes would also have profound effect on brain development and maturation. Considerable evidence indicates that dysfunction during neurodevelopment contributes to pathogenesis of schizophrenia (Bloom F. E., Arch Gen Psychiatry, 50: 224-227 (1993); Weinberger D. R., Arch Gen Psychiatry, 44: 660-669 (1987)), and specific proteins associated with development processes, reelin and GAP-43, have been found at abnormal levels in schizophrenic and bipolar subjects (Perrone-Bizzozero et al., PNAS, 93: 14182-14187 (1996)). By means of binding to AA, apoD could also affect developmental processes. Arachidonic acid acts as a second messenger in several neurotransmitter systems, including the action of basic fibroblast growth factors that are critical for normal brain development. Synaptic organization would also dependent upon the integrity of the membrane structure. Recent studies have demonstrated increases in various presynaptic proteins (Gabriel et al., Arch Gen Psychiatry, 54: 559-566 (1997)), and synapsin and synaptophysin, two synaptic vesicle-associated proteins (Browning et al., Biol Psychiatry, 34: 529-535 (1993); Honer et al., Neurosci, 91: 1247-1255 (1999), in cerebral cortex of schizophrenic subjects.

In addition to measuring apoD expression in the brains of schizophrenic and bipolar human subjects compared with control human subjects, we have also measured apoD levels in serum samples using Western blot and ELISA analyses. The apoD levels were measured in serum samples of schizophrenic subjects and from brain tissue obtained post-mortem from schizophrenic and bipolar subjects and subjects with no history of psychiatric illness (controls) using both Western blot and ELISA analyses. ApoD concentrations were determined in the serum from consenting neuroleptic-free patients, patients receiving typical neuroleptic drugs and patients enrolled-in the clozapine monitoring system at the Mental Health Research Institute. Patients were deemed neuroleptic free if they had not received neuroleptic drugs orally for 1 month or by depot injection for 3 months before blood collection. The schizophrenic patients consisted of 24 males and 8 females. Normal volunteers consisted of 13 males and 17 females made up of staff members of the Mental Health Research Institute. There were no significant differences in the ages of the schizophrenic subjects and the control subjects (schizophrenic subjects: mean age=35±10; control subjects: mean age=30±7).

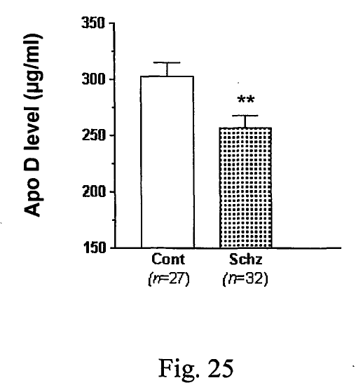

Using two different antibodies to human apoD, ELISA was performed to quantify apoD concentrations in serum samples from normal subjects and patients with schizophrenia. ApoD concentrations were measured with ELISA using purified apoD as a standard. As shown in FIG. 25, a significant decrease in the concentration of apoD was observed in schizophrenic patients relative to control subjects (256 μg/ml±11 versus 303 μg/ml±12; p=0.0083). The number of subjects in each group is indicated in the parentheses.

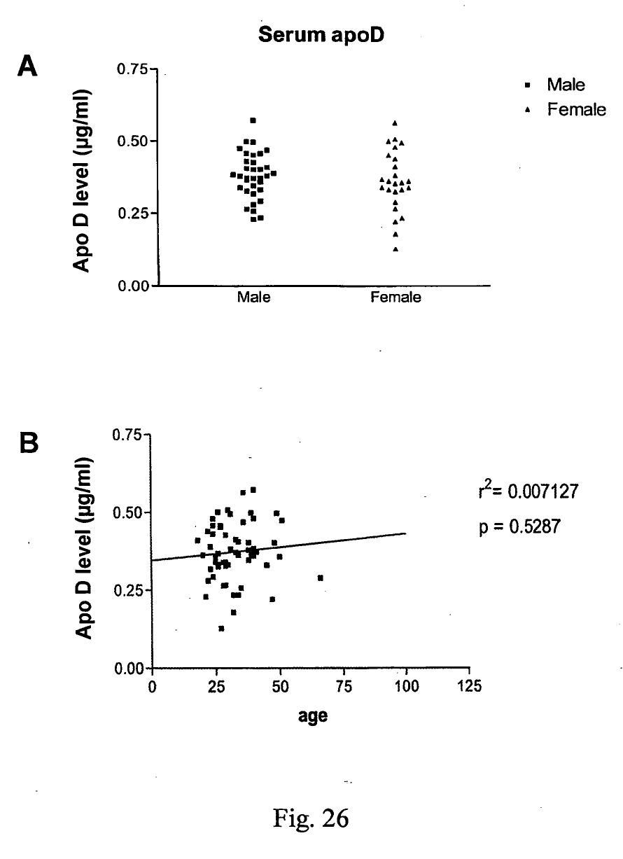

Additional studies have shown that there is no correlation between apoD levels and gender (FIG. 26A), or apoD levels and age (FIG. 26B).

These studies have shown a decrease in the serum levels of apoD in schizophrenic patients, suggesting a systemic deficiency in pathways associated with apoD. Numerous other studies have reported differences of total membrane phospholipid content, fatty acid content and cholesteryl esters in membranes from erythrocytes, red blood cells and fibroblasts and in frontal cortex of schizophrenic patients (Mahadik et al., Schizophrenia Res, 13: 239-247 (1994); Keshaven et al., J Psychiatry Res, 49: 89-95 (1993); Sengupta et al., Biochem Med, 25: 267-275 (1981); Stevens, J. D., Schizophr Bull, 6: 60-61 (1981); Horrobin et al., Biol Psychiatry, 30: 795-805 (1991); Peet et al., Prostaglandins Leukot Essent Fatty Acids, 55: 71-75 (1996)). Decreased concentrations of essential fatty acids, especially arachidonic acid (AA), in schizophrenic patients have been replicated in several studies. For example, low levels of AA-enriched phospholipids have been observed in cultured fibroblasts in chronic and in first episode schizophrenic patients (Mahadik et al., Schizophrenia Res, 13: 239-247 (1994); Mahadik et al., Psychiatry Res, 63: 133-142 (1996)). Studies have also demonstrated a marked depletion of AA in red blood cells and an abnormal incorporation/esterification of AA into platelet membranes of patients with schizophrenia (Peet et al., Prostaglandins Leukot Essent Fatty Acids, 55: 71-75 (1996); Demisch et al., Prostaglandins Leukot Essent Fatty Acids, 46: 47-52 (1992); Yao et al., Psychiatry Res, 60: 11-21 (1996)). These alterations in fatty acid concentrations are consistent with the increases levels of PLA₂ activity detected in the serum and cortex of schizophrenic patients (Ross et al., Arch Gen Psychiatry, 54: 487-494 (1997); Gattaz et al., Biol Psychiatry, 22: 421-426 (1997); Ross et al., Brain Research, 821: 407-413 (1999)). It has also been suggested that a defect in the transport of dietary fatty acids is associated with the pathophysiology of schizophrenia (Glen et al., Schizophr Res, 12: 53-61 (1994)). In view of the reported ability of apoD to bind AA, it is possible that the decreases serum apoD levels observed in this study result from pre-existing AA and/or phospholipid deficiencies.

In summary, we have shown that apoD levels are low in the serum of schizophrenic subjects, but elevated in the dorsolateral prefrontal cortex and caudate of schizophrenic and bipolar subjects. Although the specific functions of apoD in the CNS and in psychiatric illnesses remain unclear, we suggest that apoD may be a compensatory region-specific marker for a neuropathological process that is initiated because of systemic lipid metabolism insufficiencies.

### EXAMPLE 6

### Characterization of ApoD in PDAPP Mice

The mouse studies described above (Example 4) showed that apoD is regulated by chronic antipsychotic drug administration, and Example 5 described an increase in apoD expression in the prefrontal cortex of schizophrenic and bipolar human subjects. The combined results suggest that apoD is a marker for neuropathology associated with psychiatric disorders and therefore can be used to target abnormalities in specific anatomical brain regions. This example describes studies investigating a potential role for apoD in the neuropathology of Alzheimer's disease.

This example describes the measurement of ApoD mRNA expression in transgenic mice expressing mutated human amyloid precursor protein under control of platelet-derived growth factor promoter (PDAPP mice), and the findings suggest that, although increases in apoD expression are a normal feature of brain aging, super-increases may represent a glial cell compensatory response to beta-amyloid deposition in Alzheimer's disease.

Alzheimer's disease (AD) is characterized by progressive neurodegeneration and cognitive impairment accompanied by formation of senile plaques, neurofibrillary tangles and neuronal loss (Terry et al., 1994). The senile plaques in AD contain amyloid β (Aβ) protein, which is derived from the amyloid β precursor protein (APP) (Selkoe et al., 1988, Selkoe, 1993), and are associated with reactive astrocytes and microglia (Terry et al., 1964, Dickson et al., 1988, Wisniewski et al., 1989). The pathophysiological role for these plaques in AD is not clear. Several additional proteins are also aggregated with amyloid plaques, including apolipoprotein E (apoE), a 34 kDa very low-density protein that has been implicated in the pathogenisis of AD. In humans, three major allelic variations in the apoE gene (ε2, ε3 and ε4) exist and these encode three protein isoforms. It is now well established that inheritance of the ε4 allele greatly increases the risk for developing late-onset familial and sporatic AD (Strittmatter et al., 1993). In addition to the genotyping studies, apoE mRNA and protein levels have been shown to be elevated in brains of Alzheimer's subjects (Yamada et al., 1995, Yamagata et al., 2001), and apoE immunoreactivity has been localized not only to the senile plaques, but also to vascular amyloid and the neurofibrillary tangles of AD (Poirier, 2000). It has also been demonstrated that apoE is essential for beta-amyloid deposition in a mouse model of AD (Bales et al., 1999).

In addition to apoE, other apolipoproteins have been implicated in AD suggesting perhaps a common pathway of lipid homeostasis in the pathology and progression of the disease. For example, the amyloid plaques in AD patients have also been shown to be immunoreactive for apoA-1, apoJ and apoB (Harr et al., 1996, Namba et al., 1992, Calero et al., 2000). In addition, increased levels of apoJ have been detected in the cortex and hippocampus of Alzheimer's subjects (Lidstrom et al., 1998) and abnormal levels of apoB and apoA-l have been reported in plasma from Alzheimer's subjects (Caramelli et al., 1999, Merched et al., 2000).

ApoD has also been associated with AD (Kalman et al., 2000, Belloir et al., 2001, Terrisse et al., 1998). ApoD is a 29 kDa glycoprotein that, like apoE and apoJ, is synthesized in cells within the CNS (reviewed by Rassart et al., 2000). However, unlike other apolipoproteins, which have a common amphipathic α-helical protein structure, apoD is composed primarily of antiparallel β-sheets, hence shares a similar structure to the lipocalin superfamily of lipid-binding proteins (Rassart et al., 2000). Accordingly, apoD has been shown to bind hydrophobic molecules, such as steroid hormones, retinoids, heme-related compounds and arachidonic acid (Rassart et al., 2000). The function of apoD in the CNS is not clear, but it is thought to function in maintenance and repair after CNS insult or in response to CNS pathology. Recent studies have demonstrated increased levels of apoD in the CSF of AD and other neurological disorders and increases in the hippocampus and cortex of AD subjects (Terrisse et al., 1998, Kalman et al., 2000, Belloir et al., 2001). Examples 4 and 5 of the current embodiment demonstrated increased apoD expression in prefrontal cortex and caudate of schizophrenia and bipolar disorder subjects (Thomas et al., 2001b). These cumulative findings have led to the hypothesis that apoD is a marker for brain regions that undergo neuropathology as a component of various human neurological disorders.

To date, several transgenic mice overexpressing the human APP gene or APP mutations have been developed (Higgins et al., 1994, Mucke et al., 1994, Games et al., 1995)). One model, the PDAPP mouse, harbors a mutation in APP directing a valine to phenylalanine change at position 717 (APPV717F) resulting in an overproduction of the highly amyloidogenic Aβ (1-42) relative to other Aβ peptides. This mouse exhibits many prominent age-dependent pathological and behavioral features of AD, including progressive neuropathology, amyloid beta deposition, neuritic plaques, astrocytosis and microgliosis and synaptic loss (Games et al., 1995, Chen et al., 1998, Chen et al., 2000, Masliah et al., 1996). To further explore the potential of a role for apoD in the neuropathology of Alzheimer's disease, we have measured apoD mRNA expression in brains of young and aged PDAPP transgenic mice.

Transgenic mice expressing human mutant APP (APPV717F) have been described previously (Games et al., 1995, Chen et al., 1998). These mice were developed using a platelet-derived growth factor promoter driving a hβAPP minigene encoding the 717_{V→F} mutation associated with familial AD (Games et al., 1995). Mice were fifth generation female heterozygous PDAPP line 109 mice produced on a Swiss Webster X B₆D₂F₁ (C57BL/6× DBA/2) outbred background (Games et al., 1995, Masliah et al., 1996). For this study, aged (26 month) and young (6 month) groups of female PDAPP mice and non-Tg littermates (n=4/group) were utilized (provided by Elan Pharmaceuticals). These mice exhibit many prominent age-dependent pathological features of AD, including progressive neuropathology, amyloid beta deposition, neuritic plaques, gliosis and decreased synaptic and dendritic densities (Chen et al., 2000, Chen et al., 1998, Masliah et al., 1996, Games et al., 1995).

Given previous studies demonstrating involvement of apolipoproteins in AD and particularly the hypothesis that apoD is a marker for neuropathology, we performed in situ hybridization analysis on young and aged PDAPP transgenic animals to determine the spectrum of apoD mRNA expression in this Alzheimer's mouse model. Briefly, mice were anesthetized with halothane and perfused with cold phosphate buffered saline (PBS; pH 7.5) followed by 4% paraformaldehyde in PBS. Brains were removed and post-fixed in 4% paraformaldehyde overnight, then cryoprotected in 30% sucrose in paraformaldehyde for 24 hr. In situ hybridization was performed on free-floating sections as described previously (Example 2).

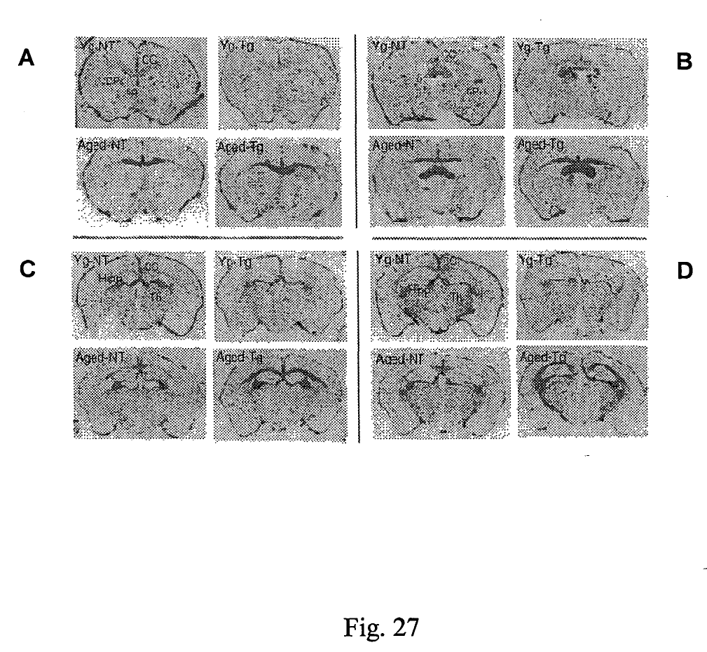

Regional ApoD gene expression in the brain was compared in young nontransgenic (Yg-NT), young transgenic (Yg-Tg), aged nontransgenic (Aged-N) and aged transgenic (Aged-Tg) mice (FIG. 27A-D). In the brains of several species, apoD expression has previously been found primarily in white matter regions, pial cells surrounding the brain and perivascular cells scattered throughout the brain (Boyles et al., 1990, Thomas et al., 2001 a, Drayna et al., 1986). We observed a similar pattern of apoD expression in our young control mice (Yg-NT, FIG. 27A-D). At the gross level, we did not detect substantial increases in apoD expression in young PDAPP transgenic mice (Yg-Tg) compared to young controls (Yg-NT; see top two images going across in each panel FIG. 27A-D). However, both aged controls (Aged-NT) and aged PDAPP transgenic mice (Aged-Tg) displayed robust increases in expression in several areas compared to young mice (see lower two images going across in each panel FIG. 27A-D). These increases in expression were most notable in the white matter tracts: hippocampal fimbria (fi), corpus callosum (cc), septal white matter tracts (sp). Comparison between aged wild type and PDAPP mice revealed that the PDAPP mice had greater apoD expression as compared to the wild type. Representative sections were taken at four different levels. In summary, FIG. 27A represents tissue slices taken at the level of the caudatoputamen (CP), demonstrating gene expression in the corpus callosum (cc) and septal white matter tracts (sp). FIG. 27B represents tissue slices taken at the level of of the globus pallidus (GP) demonstrating gene expression in the hippocampal fimbria (fi) and corpus callosum (cc). FIG. 27C,D represent tissue slices taken at-the level of the hippocampus (Hipp) and thalamus (Th) demonstrating gene expression in the corpus callosum (cc).

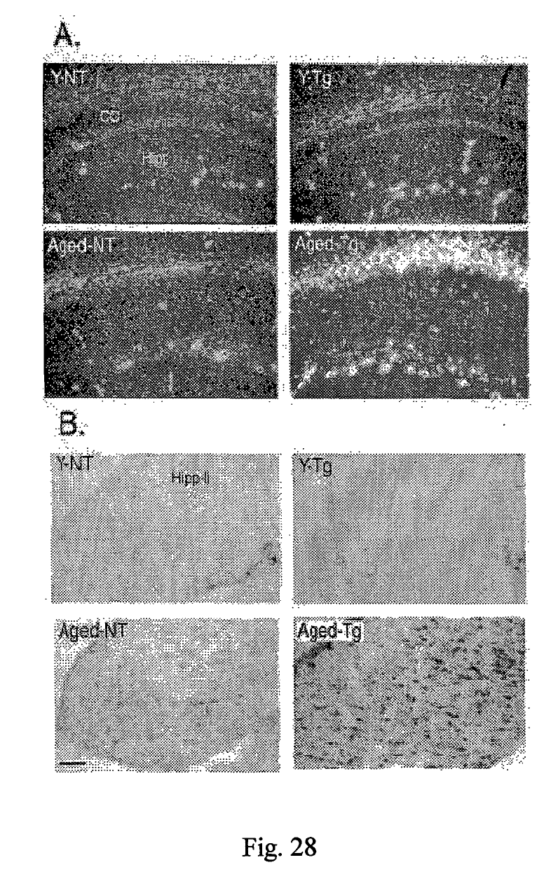

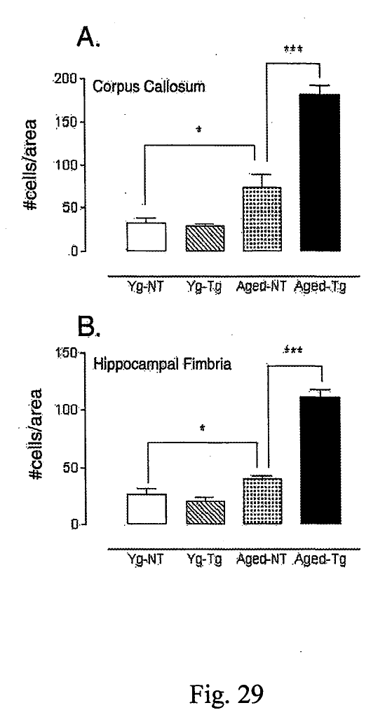

At high magnification, the abundance of apoD hybridization signals within individual glial cells in the hippocampal fimbria and corpus callosum of the age PDAPP animals was striking (FIGS. 28, 29). ApoD quantification in the corpus callosum and fimbria was performed by counting the number of cells determined positive for apoD expression within a defined field of view. Cell counts were performed at 20× magnification in both brightfield and darkfield on 4 different brain slices from both the corpus callosum (FIG. 29A) and fimbria (FIG. 29B) of each animal. One-way analysis of variance with a Bonferroni post-test was used to determine significant differences among young and aged transgenic and non-transgenic animals. An approximate 300% increase in apoD-positive cells was observed in both the corpus callosum (FIG. 28A, 29A) and hippocampal fimbria (FIG. 28B, 29B) of aged-transgenic animals vs. aged controls (p<0.001). In non-transgenic mice, 159 and 217% increases were observed in corpus callosum and fimbria, respectively, in the aged vs. young animals (p<0.05) (FIGS. 28 and 29). 447 and 613% increases were detected in corpus callosum and fimbria, respectively, in the aged transgenic vs. young transgenic animals (p<0.0001) (FIGS. 28 and 29). The cell types expressing elevated levels of apoD in these regions were identified, based on size, morphology and previous co-localization studies (Example 4), as oligodendrocytes and astrocytes. Evidence for microglial expression of apoD was less clear. High magnification views also revealed increased apoD expression in cells of the hippocampus of some of the young and old transgenic mice versus their respective controls (FIG. 28).

This study demonstrated increases in apoD expression in aged control and aged PDAPP transgenic mice, predominantly in the hippocampal fimbria and corpus callosum, indicating an association of apoD with not only age-related processes, but also pathological processes accompanying expression of the mutant amyloid precursor protein. The PDAPP mouse strain exhibits many prominent age-dependent pathological and behavioral features of AD, including progressive neuropathology, amyloid beta deposition, neuritic plaques, astrocytosis and microgliosis, synaptic loss and deficits in learning and memory (Games et al., 1995, Chen et al., 1998, Masliah et al., 1996). Many of the neurodegenerative changes in the PDAPP mice are observed in the hippocampus and cortex, two brain structures implicated in the pathophysiology of AD. Aβ deposits and aggregates begin to form in the PDAPP mice at around 8 months and, by one year of age, Aβ deposits are common in the hippocampus and in the frontal and cingulate cortices (Johnson-Wood et al., 1997). These mice also develop behavioral deficits that are likened to the cognitive decline seen in AD (Chen et al., 2000). Although AD is generally considered to affect grey matter regions, histological studies have demonstrated pathological changes in white matter (Brun and Englund, 1986, Rose et al., 2000). Loss of white matter integrity can be responsible for loss of connectivity in AD and consequential decline in cognitive functions (Brun and Englund, 1986). The corpus callosum is the largest white matter structure in the brain connecting the neocortex of each side and is responsible for normal interhemispheric communication. The hippocampal fimbria is another white matter structure important for connectivity of the hippocampus. Afferents from the septum enter the hippocampal formation via the fimbria and are distributed to both the hippocampus and dentate gyrus. Accordingly, increases in apoD expression in the septal white matter tracts were also observed. Super-expression of apoD in the white matter regions may have several implications regarding the effect of amyloid plaques in the pathology of AD.

A commonly observed feature of AD is the presence of activated astrocytes and microglia, which also is a common feature of other neurodegenerative disorders and CNS pathology. Gliotic changes are observed in PDAPP mice similar to those observed in AD (Games et al., 1995, Chen et al., 1998). Aβ peptides, including Aβ1-42, can induce profound glial cell activation, suggesting that the Aβ deposition in this model may be responsible for the observed gliotic effects. The increased expression of apoD in astrocytes may indicate a response associated with Aβ deposition-induced astrocytosis. In addition, apoD is elevated in response to microglial activation (Monica Carson, personal communication). Previous studies have demonstrated apoD induction in response to CNS pathology and in human neurological disorders. For example, increased apoD immunoreactivity and mRNA levels have been observed in glial cells and neurons of the hippocampus and/or cortex after kainic acid and traumatic brain injury (Franz et al., 1999, Ong et al., 1997). Increased levels of apoD mRNA protein expression have been observed in the hippocampus after entorhinal cortex lesioning (Terrisse et al., 1999), which results in reactive synaptogenesis and compensatory glial functions. Recent studies have demonstrated increased levels of apoD in the CSF of AD and other neurological disorders and increases in the hippocampus and cortex of AD subjects (Terrisse et al., 1998, Kalman et al., 2000, Belloir et al., 2001). The increases in apoD expression observed in the hippocampus of both young and old transgenic mice in the present study are consistent with these studies. Increased apoD levels in astrocytes and microglia are consistent with a compensatory response to Aβ-induced CNS pathology. It is possible that apoD is elevated to counteract detrimental effects of amyloid deposition. This supports the theory that Aβ deposition has neurodegenerative consequences, as opposed to neuroprotective effects, as others have suggested (Campbell, 2001).

In the PDAPP transgenic mice, apoD expression was elevated in oligodendrocytes, which are the cells responsible for myelin synthesis. ApoD expression in these cells may reflect a dysfunction in myelin or axonal integrity resulting from Aβ deposition and plaque formation. This is consistent with white matter deficits associated with AD. Histological studies have shown pathological changes, such as a loss of axons and oligodendrocytes together with a reactive astrocytosis in the white matter regions of AD subjects (Rose et al., 2000, Brun and Englund, 1986). These deficits are related to a loss of connectivity in AD and have been associated with the decline of cognitive functions observed in AD (Rose et al., 2000, Brun and Englund, 1986). Thus, Aβ aggregation and deposition may initiate the white matter deficits observed in AD patients, hence accounting for the cognitive dysfunction.

The putative neuroprotectant, compensatory functions of apoD in the CNS are consistent with proposed functions for other apolipoproteins in AD, including apoE, apoA1 and apoJ (Poirier, 2000, Calero et al., 2000). These functions are thought to involve cholesterol turnover, which is increased in glial cells and neurons during neuron repair and membrane remodelling. It has also been suggested that several members of the apoprotein family may interact with Aβ deposits in senile plaques through a common amphipathic alpha-helical domain (Harr et al., 1996). While apoD may have a similar function in the CNS as the other apoproteins, it does not share a similar protein structure as the other family members and does not bind cholesterol with high affinity, hence may have a unique effect via binding of different ligands. ApoD may be involved in the binding of steroids or fatty acids released upon CNS insult, or the transport of lipid molecules necessary for cellular regeneration, and therefore may function in CNS maintenance and tissue repair.

We also observed an increase in apoD expression in aged vs. young control animals. This was observed primarily in the white matter regions, corpus callosum and hippocampal fimbria. These findings are in agreement with Kalman et al., (Kalman et al., 2000) that have shown increases in astrocytic expression of apoD in cortical regions of aged, post-mortem human subjects (68-83 years old). Loss of white matter is also thought to be a major factor in the cognitive decline associated with aging (Fazekas et al., 1998).

In summary, we have detected increases in apoD mRNA expression in brains from aged PDAPP transgenic mice, one of several Alzheimer's disease mouse models. These results implicate lipid homeostatic mechanisms in white matter in the pathological processes associated with Alzheimer's disease, as well as in normal aging, and suggest that Aβ aggregation/deposition may initiate the white matter deficits observed in AD patients, hence accounting for the cognitive dysfunction.

This example, in combination with Examples 4 and 5, suggest that apoD is a marker for neuropathology associated with psychiatric disorders and therefore can be used to target abnormalities in specific anatomical brain regions.

### EXAMPLE7

### Characterization of Other Clozapine Regulated DSTs

The same experimental paradigm used in Example 1 for clozapine treatment was used for the TOGA analyses. This experiment describes the validation of two additional DSTs by Northern analysis (CLZ_{—}38 and CLZ_{—}44), and characterization of expression patterns by in situ hybridization on 10 DSTs (CLZ 3, CLZ_{—}16, CLZ_{—}17, CLZ_{—}24, CLZ_{—}26, CLZ_{—}28, CLZ_{—}34, CLZ_{—}38, CLZ_{—}44, CLZ_{—}64) in mouse CNS. In situ hybridization was performed on free-floating coronal sections (25 μM thick) with an ³⁵S-labeled, single-stranded antisense cRNA probe specific for each DST using the methods described in the above examples. The regional distributions for these are summarized in Table 9.

A subset of clones exhibit expression in specific brain regions, hence are of particular interest. These include CLZ 3, CLZ_{—}17, CLZ_{—}38, CLZ_{—}40 (Example 3), CLZ_{—}43 (Example 1), and CLZ_{—}44. In addition to having ubiquitous low abundant expression, three additional clones, CLZ 24, CLZ_{—}26 and CLZ 28, displayed relatively enriched expression in the cortex. Since specific expression of genes in a certain brain region reflects an association with the functional specialization of the region, these studies are useful to determine the role of specific genes and their contribution to brain function. For example, the striatum (dorsal striatum) is responsible for motor and movement functions, while the nucleus accumbens (ventral striatum) and other limbic regions are involved in cognitive and emotional behavior, as well as reward and reinforcement. Thus, the identification of genes that are specifically expressed in a particular brain region will elucidate the mechanisms of brain function.

CLZ 3

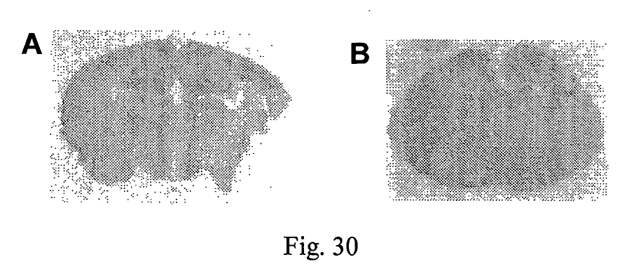

As shown in Table 1, the results of TOGA analysis indicate that CLZ_{—}3 (SEQ ID NO: 1) is up-regulated by clozapine treatment. Table 2 shows that CLZ_{—}3 is a serine protease HTRA mRNA. In further characterization of CLZ_{—}3, in situ hybridization analysis using an antisense cRNA probe directed against the 3′ end of CLZ_{—}3 were performed. In situ hybridization was performed on free-floating coronal sections (25 μM thick) with an ³⁵S-labeled, single-stranded antisense cRNA probe of CLZ_{—}3 using the methods described in the above examples. FIG. 30A-B is an in situ hybridization analysis using an antisense cRNA probe directed against the 3′ end of CLZ_{—}3, showing the pattern of CLZ_{—}3 mRNA expression in a coronal section through the hemispheres at level of hippocampus (FIG. 30A) and cross section through midbrain (FIG. 30B) in mouse brain. As shown in FIG. 30A and B, CLZ_{—}3 mRNA is expressed in the cortex, thalamus, hippocampus, striatum, and amygdala.

CLZ 16

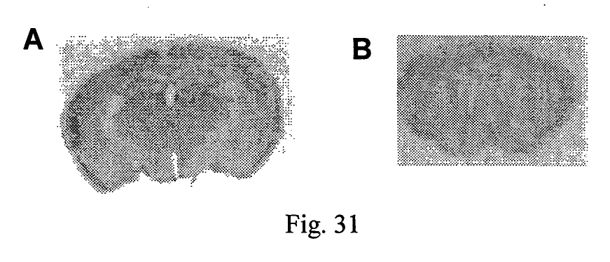

As shown in Table 1, the results of TOGA analysis indicate that CLZ_{—}16 (SEQ ID NO: 15) is slightly down-regulated by clozapine treatment. Table 2 shows that CLZ_{—}16 is an arm-repeat protein. In further characterization of CLZ_{—}16, in situ hybridization analysis using an antisense cRNA probe directed against the 3′ end of CLZ_{—}16 were performed to show the pattern of CLZ_{—}16 mRNA expression in mouse anterior brain (FIG. 31B) and posterior brain (FIG. 31A). As shown in FIG. 31A and B. CLZ_{—}16 mRNA is expressed ubiquitously throughout mouse brain. FIG. 31A shows dense labelling in the cortex and surrounding the hippocampal formation as well as moderate labelling in the dorsal thalamus and posterior brain. FIG. 31B shows uniform labelling throughout.

CLZ 17

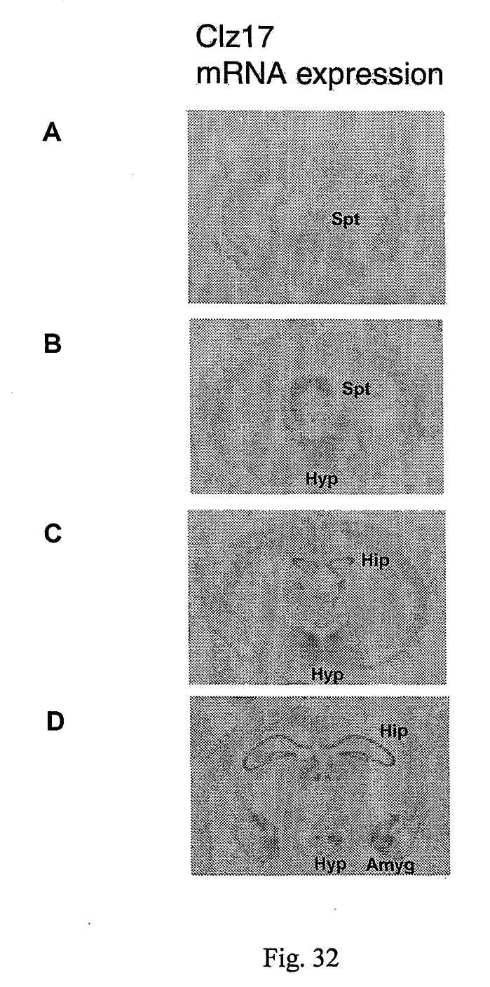

As shown in Table 1, the results of TOGA analysis indicate that CLZ_{—}17 (SEQ ID NO: 28) is slightly down-regulated by clozapine treatment. Table 3 shows that CLZ_{—}17 matches several ESTs isolated from mouse tissue. In further characterization of CLZ_{—}17, in situ hybridization analysis using an antisense cRNA probe directed against the 3′ end of CLZ_{—}17 were performed. CLZ_{—}17 expression was observed in the septal nucleus (SPT), in the hypothalamic nuclei (HYP), in the hippocampus (HIP), and amygdala (AMYG) (FIG. 32A-D).

CLZ 24

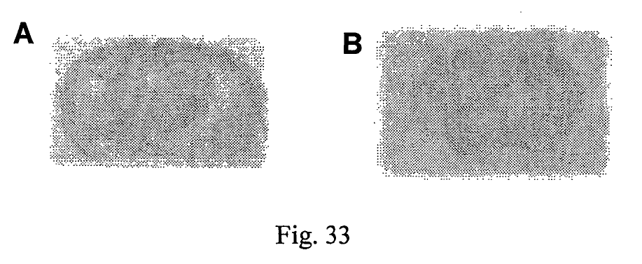

As shown in Table 1, the results of TOGA analysis indicate that CLZ_{—}24 (SEQ ID NO: 7) is up-regulated by clozapine treatment. Table 2 shows that CLZ_{—}24 is an EST isolated from rat tissue. In further characterization of CLZ_{—}24, in situ hybridization analysis using an antisense cRNA probe directed against the 3′ end of CLZ_{—}24 were performed to show the pattern of CLZ_{—}24 mRNA expression in mouse anterior brain (FIG. 33B) and posterior brain (FIG. 33A). FIG. 33A-B shows an in situ hybridization analysis using an antisense cRNA probe directed against the 3′ end of CLZ_{—}24, showing the pattern of CLZ_{—}24 mRNA expression in a coronal section through the hemispheres (FIG. 33A) and cross section through the brainstem (FIG. 33B) in mouse brain. As shown, CLZ_{—}24 mRNA is ubiquitously expressed in the cortex.

CLZ 26

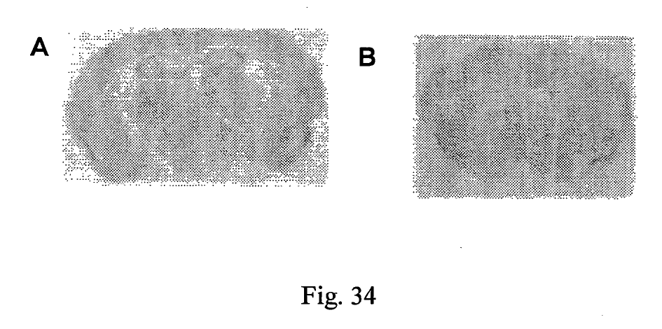

As shown in Table 1, the results of TOGA analysis indicate that CLZ_{—}26 (SEQ I D NO: 29) is slightly down-regulated by clozapine treatment. Table 3 shows that CLZ_{—}26 is a metalloprotease-disintegrin MDC15 mRNA. In further characterization of CLZ_{—}26, in situ hybridization analysis using an antisense cRNA probe directed against the 3′ end of CLZ_{—}26 were performed to show the pattern of CLZ_{—}26 mRNA expression in mouse anterior brain (FIG. 34B) and posterior brain (FIG. 34A). In situ hybridization was performed using the methods described in the above examples. FIG. 34A-B is an in situ hybridization analysis using an antisense cRNA probe directed against the 3′ end of CLZ_{—}26, showing the pattern of CLZ_{—}26 mRNA expression in a coronal section of the hemispheres at the level of hippocampal formation (FIG. 34A) and coronal section of the hemispheres at the level of striatum (FIG. 34B) in mouse brain. As shown, CLZ_{—}26 mRNA is ubiquitously expressed in the cortex.

CLZ 28

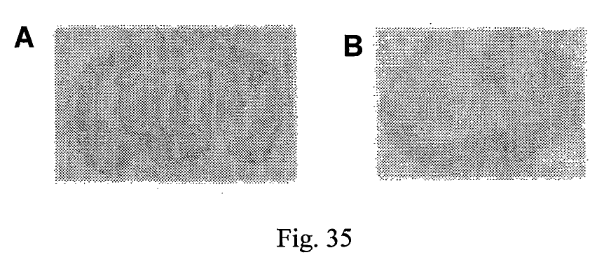

As shown in Table 1, the results of TOGA analysis indicate that CLZ_{—}28 (SEQ ID NO: 30) is down-regulated by clozapine treatment. Table 3 shows that CLZ_{—}28 matches several ESTs isolated from mouse tissue. In further characterization of CLZ_{—}28, in situ hybridization analysis using an antisense cRNA probe directed against the 3′ end of CLZ_{—}28 were performed to show the pattern of CLZ_{—}28 mRNA expression in mouse anterior brain (FIG. 35B) and posterior brain (FIG. 35A). In situ hybridization was performed using the methods described in the above examples. Figure XA-B is an in situ hybridization analysis using an antisense cRNA probe directed against the 3′ end of CLZ_{—}28, showing the pattern of CLZ_{—}28 mRNA expression in a coronal section through the hemispheres at the level of hippocampus (FIG. 35A) and coronal section through the posterior region of hemispheres (FIG. 35B) in mouse brain. As shown in FIG. 35A and B, CLZ_{—}28 mRNA is expressed ubiquitously in the cortex.

CLZ 34

As shown in Table 1, the results of TOGA analysis indicate that CLZ_{—}34 (SEQ ID NO: 9) is upregulated with clozapine treatment at 45 minutes and 7 hours, but decreases to control level by day 5 and remains at about control level for as long as 12 days, showing a slight increase at day 14. CLZ_{—}34 corresponds with GenBank sequence U08262, which is identified as a rat N-methyl-D-aspartate receptor/NMDAR1-2a subunit (NMDAR1) (Table 2 and 3). The NMDAR1 receptor is a glutamate receptor involved in the processes underlying learning and memory. In addition, numerous studies show that blockade of glutamate actions by noncompetitive (e.g. MK801 and dextromethorphan) and competitive (e.g. LY274614). NMDA receptor antagonists blocks or reduces the development of morphine tolerance following long term opiate administration (Trujillo et al., Science, 251, 85-87, (1991); Elliott et al., Pain, 56, 69-75 (1994); Wiesenfeld-Hallin, Z., Neuropsychopharm., 13, 347-56 (1995)). The early change in the level of expression of CLZ_{—}34 which has high homology with an NMDA receptor is interesting in view of the ability of NMDA antagonists to block the development of tolerance to opioids.

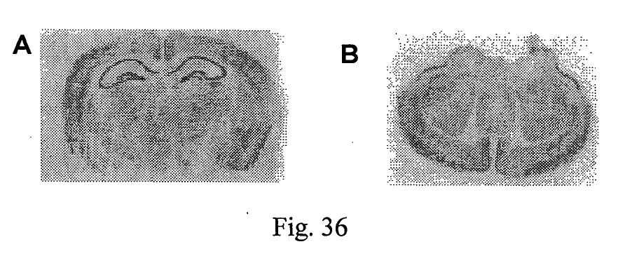

In further characterization of CLZ 34, in situ hybridization analysis using an antisense cRNA probe directed against the 3′ end of CLZ_{—}34 were performed to show the pattern of CLZ_{—}34 mRNA expression in mouse anterior brain (FIG. 36B) and posterior brain (FIG. 36A). In situ hybridization was performed using the methods described in the above examples. FIG. 36A-B is an in situ hybridization analysis using an antisense cRNA probe directed against the 3′ end of CLZ_{—}34, showing the pattern of CLZ_{—}34 mRNA expression in a coronal section through the hemispheres at the level of hippocampus (FIG. 36A) and cross section through the midbrain (FIG. 36B) in mouse brain. As shown in FIG. 36A and B, CLZ_{—}34 mRNA is ubiquitously expressed.

CLZ 38

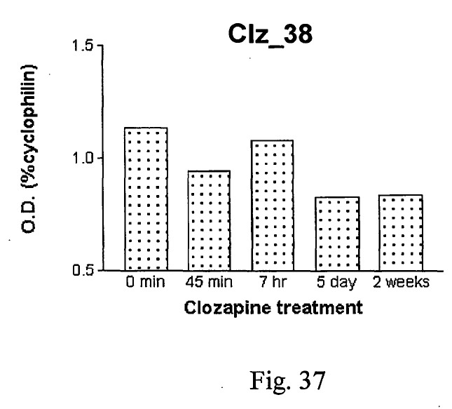

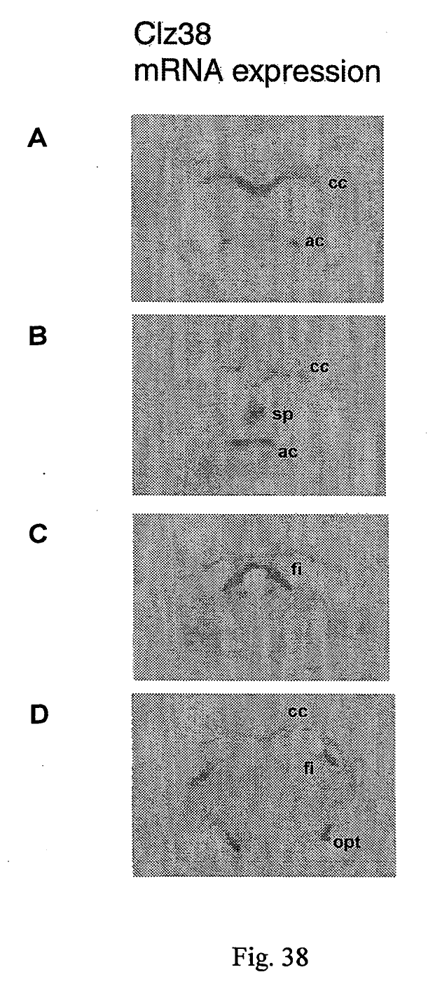

Table 2 shows that CLZ_{—}38 (SEQ ID NO: 11) is an oligodendrocyte-specific protein mRNA. In further characterization of CLZ_{—}38, Northern blot analyses were performed to determine the pattern of expression in the striatum/nucleus accumbens of control mice and mice treated with clozapine for 45 minutes, 7 hours, 5 days, and 2 weeks. FIG. 37 is a graphical representation of the described Northern blot analyses. As shown, the pattern of CLZ_{—}38 expression in clozapine-treated animals was similar to the pattern observed with TOGA analysis. CLZ_{—}38 mRNA expression in the brain was determined by in situ hybridization using riboprobes specific to the DST (FIG. 38A-D). CLZ_{—}38 expression was observed primarily in the white matter tracts of the brain. FIG. 38A,B demonstrates CLZ_{—}38 expression in the corpus callosum (cc) and anterior commisure (ac). FIG. 38B demonstrates expression in the white matter of the septum (sp). FIG. 38C demonstrates CLZ_{—}38 expression by cells in the hippocampal fimbria (fi). FIG. 38D demonstrates CLZ_{—}38 expression in the cc, fi, and optic tract (opt).

CLZ 44

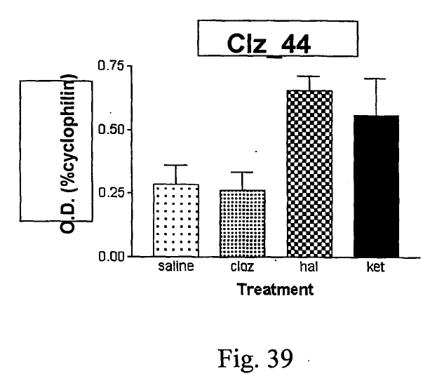

As shown in Table 1, the results of TOGA analysis indicate that CLZ_{—}44 (SEQ ID NO: 38) is up-regulated by clozapine treatment. Table 2 shows that CLZ_{—}44 matches an EST isolated from mouse tissue. In further characterization of CLZ_{—}44, Northern blot analyses were performed to determine the pattern of expression in the striatum/nucleus accumbens after 2 weeks of treatment of control mice, clozapine-treated mice, haloperidol-treated mice, and ketanserin-treated mice (FIG. 39). Ketanserin is a 5HT_{2A/2C}—selective antagonist, and is used to determine serotonorgic involvement in these drug effects. FIG. 39 is a graphical representation of the described northern blot analyses. As shown, after 2 weeks of treatment, CLZ_{—}44 was up-regulated with haloperidol and ketanserin, but not clozapine. This suggests that both dopamines D2 and 5HT_{2A/2C} receptors are involved in CLZ_{—}44 expression regulation. The lack of effect of clozapine may indicate that antagonism at other receptors (i.e. 5HT₃, D4, D1) may override the effects of D2, 5HT₂ receptors.

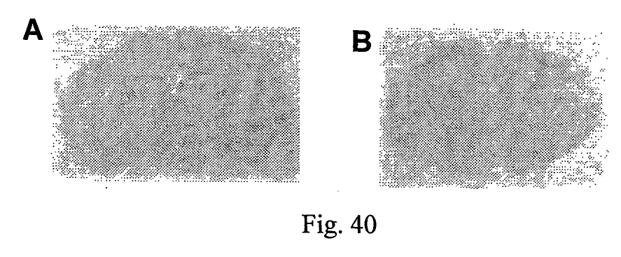

In further characterization of CLZ_{—}44, in situ hybridization analysis using an antisense cRNA probe directed against the 3′ end of CLZ_{—}44 were performed to show the pattern of CLZ_{—}44 mRNA expression in mouse anterior brain (FIG. 40A) and posterior brain (FIG. 40B). FIG. 40A-B is an in situ hybridization analysis using an antisense cRNA probe directed against the 3′ end of CLZ_{—}44, showing the pattern of CLZ_{—}44 mRNA expression in a coronal section showing labelling in the hippocampus, hypothalamus, and temporal cortex (FIG. 40A) and coronal section showing cortical labelling (FIG. 40B) in mouse brain.

CLZ 64

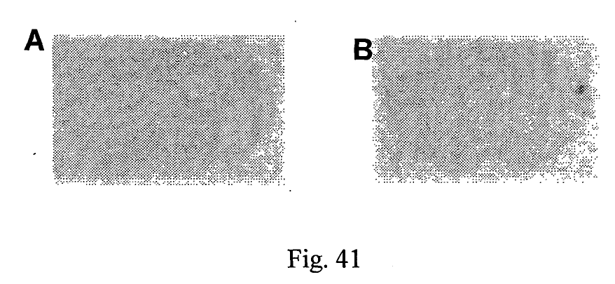

As shown in Table 1, the results of TOGA analysis indicate that CLZ_{—}64 (SEQ ID NO: 48) is up-regulated by chronic clozapine treatment. Table 2 shows that CLZ_{—}64 matches an EST isolated from mouse tissue and shares homolgy with mitochondrial enoyl-CoA hydratase mRNA. In further characterization of CLZ_{—}64, in situ hybridization analysis using an antisense cRNA probe directed against the 3′ end of CLZ_{—}64 were performed to show the pattern of CLZ_{—}64 mRNA expression in mouse anterior brain (FIG. 41B) and mid-brain (FIG. 41A). In situ hybridization was performed on free-floating coronal sections (25 AM thick) with an ³⁵S-labeled, single-stranded antisense cRNA probe of CLZ_{—}64 using the methods described in the above examples. FIG. 41A-B is an in situ hybridization analysis using an antisense cRNA probe directed against the 3′ end of CLZ_{—}64, showing the pattern of CLZ 64 mRNA expression in different coronal sections of the hemispheres in mouse brain. As shown in FIG. 41A and B. CLZ_{—}64 mRNA is ubiquitously expressed.

Summary

In summary, in situ hybridization analysis was utilized to determine the role of specific genes and their contribution to brain function. These studies demonstrate the ability of TOGA to identify genes associated with specific brain regions that could be used as tools to understand the specialized functions associated with these regions. DSTs that exhibit region specific expression could not only serve as important markers for understanding function but also drug response in the treatment of neurological disorders.

### Other Preferred Embodiments

The present invention also relates to the genes corresponding to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. The corresponding gene can be isolated in accordance with known methods using the sequence information disclosed herein. Such methods include preparing probes or primers from the disclosed sequence and identifying or amplifying the corresponding gene from appropriate sources of genomic material.

Homologues

Also provided in the present invention are homologues including paralogous genes and orthologous genes. Homologues may be isolated and identified by making suitable probes or primers from the sequences provided herein and screening a suitable nucleic acid source for the desired homologue.

Polypeptides

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art. See, e.g., Curr. Prot. Mol. Bio., Chapter 16.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification (such as multiple histidine residues), or an additional sequence for stability during recombinant production.

The polypeptides of the present invention are preferably provided in an isolated form, and preferably are substantially purified. A recombinantly produced version of a polypeptide, including the secreted polypeptide, can be substantially purified by the one-step method described in Smith & Johnson (Gene, 67:31-40, 1988). Polypeptides of the invention also can be purified from natural or recombinant sources using antibodies of the invention raised against the secreted protein in methods which are well known in the art.

Signal Sequences

Methods for predicting whether a protein has a signal sequence, as well as the cleavage point for that sequence, are available. For instance, the method of McGeoch uses the information from a short N-terminal charged region and a subsequent uncharged region of the complete (uncleaved) protein (Virus Res., 3:271-286 (1985)). The method of von Heinje uses the information from the residues surrounding the cleavage site, typically residues −13 to +2, where +1 indicates the amino terminus of the secreted protein (Nucleic Acids Res., 14:4683-4690 (1986)). Therefore, from a deduced amino acid sequence, a signal sequence and mature sequence can be identified.

In the present case, the deduced amino acid sequence of the secreted polypeptide was analyzed by a computer program called Signal P (Nielsen et al., Protein Engineering, 10:1-6 (1997), which predicts the cellular location of a protein based on the amino acid sequence. As part of this computational prediction of localization, the methods of McGeoch and von Heinje are incorporated.

As one of ordinary skill in the art would appreciate, however, cleavage sites sometimes vary from organism to organism and cannot be predicted with absolute certainty. Accordingly, the present invention provides secreted polypeptides having a sequence corresponding to the translations of SEQ ID NO:1, SEQ ID NO:2;-SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO: 18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 which have an N-terminus beginning within 5 residues (i.e., +or −5 residues) of the predicted cleavage point. Similarly, it is also recognized that in some cases, cleavage of the signal sequence from a secreted protein is not entirely uniform, resulting in more than one secreted species. These polypeptides, and the polynucleotides encoding such polypeptides, are contemplated by the present invention.

Moreover, the signal sequence identified by the above analysis may not necessarily predict the naturally occurring signal sequence. For example, the naturally occurring signal sequence may be further upstream from the predicted signal sequence. However, it is likely that the predicted signal sequence will be capable of directing the secreted protein to the ER. These polypeptides, and the polynucleotides encoding such polypeptides, are contemplated by the present invention.

Polynucleotide and Polypeptide Variants

Polynucleotide or polypeptide variants differ from the polynucleotides or polypeptides of the present invention, but retain essential properties thereof. In general, variants have close similarity overall and are identical in many regions to the polynucleotide or polypeptide of the present invention.

Further embodiments of the present invention include polynucleotides having at least 80% identity, more preferably at least 90% identity, and most preferably at least 95%, 96%, 97%, 98% or 99% identity to a sequence contained in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Of course, due to the degeneracy of the genetic code, one of ordinary skill in the art will immediately recognize that a large number of the polynucleotides having at least 85%, 90%, 95%, 96%, 97%, 98%, or 99% identity will encode a polypeptide identical to an amino acid sequence contained in the translations of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80.

Further embodiments of the present invention also include polypeptides having at least 80% identity, more preferably at least 85% identity, more preferably at least 90% identity, and most preferably at least 95%, 96%, 97%, 98% or 99% identity to an amino acid sequence contained in translations of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID.NO:36, SEQ ID NO:37, SEQ ID NO: 38,-SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Preferably, the above polypeptides should exhibit at least one biological activity of the protein. In a preferred embodiment, polypeptides of the present invention include polypeptides having at least 90% similarity, more preferably at least 95% similarity, and still more preferably at least 96%, 97%, 98%, or 99% similarity to an amino acid sequence contained in translations of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80.

Methods for aligning polynucleotides or polypeptides are codified in computer programs, including the GCG program package (Devereux et al., Nuc. Acids Res. 12:387 (1984)), BLASTP, BLASTN, FASTA (Atschul et al., J. Molec. Biol. 215:403 (1990)), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. in App. Math., 2:482489 (1981)).

When using any of the sequence alignment programs to determine whether a particular sequence is, for instance, 95% identical to a reference sequence, the parameters are set such that the percentage of identity is calculated over the full length of the reference polynucleotide and that gaps in identity of up to 5% of the total number of nucleotides in the reference polynucleotide are allowed.

A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci., 6:237-245 (1990)). The term “sequence” includes nucleotide and amino acid sequences. In a sequence alignment the query and subject sequences are either both nucleotide sequences or both amino acid sequences. The result of said global sequence alignment is presented in terms of percent identity. Preferred parameters used in a FASTDB search of a DNA sequence to calculate percent identity are: Matrix=Unitary, k-tuple=4, Mismatch Penalty=1, Joining Penalty=30, Randomization Group Length=0, and Cutoff Score=1, Gap Penalty=5, Gap Size Penalty 0.05, and Window Size=500 or query sequence length in nucleotide bases, whichever is shorter. Preferred parameters employed to calculate percent identity and similarity of an amino acid alignment are: Matrix=PAM 150, k-tuple=2, Mismatch Penalty=1, Joining Penalty=20, Randomization Group Length=0, Cutoff Score=l, Gap Penalty=5, Gap Size Penalty=0.05, and Window Size=500 or query sequence length in amino acid residues, whichever is shorter.

As an illustration, a polynucleotide having a nucleotide sequence of at least 95% “identity” to a sequence contained in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 means that the polynucleotide is identical to a sequence contained in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 or the cDNA except that the polynucleotide sequence may include up to five point mutations per each 100 nucleotides of the total length (not just within a given 100 nucleotide stretch). In other words, to obtain a polynucleotide having a nucleotide sequence at least 95% identical to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80, up to 5% of the nucleotides in the sequence contained in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 or the cDNA can be deleted, inserted, or substituted with other nucleotides. These changes may occur anywhere throughout the polynucleotide.

Similarly, a polypeptide having an amino acid sequence having at least, for example, 95% “identity” to a reference polypeptide, means that the amino acid sequence of the polypeptide is identical to the reference polypeptide except that the polypeptide sequence may include up to five amino acid alterations per each 100 amino acids of the total length of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons. For instance, a polynucleotide variant may be produced to optimize codon expression for a particular host (i.e., codons in the human mRNA may be changed to those preferred by a bacterial host, such as E. coli).

The variants may be allelic variants. Naturally occurring variants are called “allelic variants,” and refer to one of several alternate forms of a gene occupying a given locus on a chromosome of an organism (Lewin, Ed., Genes II, John Wiley & Sons, New York (1985)). These allelic variants can vary at either the polynucleotide and/or polypeptide level. Alternatively, non-naturally occurring variants may be produced by mutagenesis techniques or by direct synthesis. See, e.g., Curr. Prot. Mol. Bio., Chapter 8.

Using known methods of protein engineering and recombinant DNA technology, variants may be generated to improve or alter the characteristics of the polypeptides of the present invention. For example, polypeptide variants containing amino acid substitutions of charged amino acids with other charged or neutral amino acids may produce proteins with improved characteristics, such as decreased aggregation. As known, aggregation of pharmaceutical formulations both reduces activity and increases clearance due to the aggregate's immunogenic activity (see, e.g., Pinckard et al., Clin. Exp. Immunol. 2:331-340 (1967); Robbins et al., Diabetes, 36: 838-845 (1987); Cleland et al., Crit. Rev. Therap. Drug Carrier Sys.,10:307-317 (1993)). Similarly, interferon gamma exhibited up to ten times higher activity after deleting 8-10 amino acid residues from the carboxy terminus of this protein (Dobeli et al., J. Biotechnology, 7:199-216 (1988)).

Moreover, ample evidence demonstrates that variants often retain a biological activity similar to that of the naturally occurring protein. For example, Gayle et al. conducted extensive mutational analysis of human cytokine IL-1a (J. Biol. Chem., 268:22105-22111 (1993)). These investigators used random mutagenesis to generate over 3,500 individual IL-1 a mutants that averaged 2.5 amino acid changes per variant over the entire length of the molecule. Multiple mutations were examined at every possible amino acid position. The investigators concluded that “[m]ost of the molecule could be altered with little effect on either [binding or biological activity].” (See, Gayle et al. (1993), Abstract). In fact, only 23 unique amino acid sequences, out of more than 3,500 amino acid sequences examined, produced a protein that differed significantly in activity from the wild-type sequence. Another experiment demonstrated that one or more amino acids can be deleted from the N-terminus or C-terminus of the secreted protein without substantial loss of biological function. Ron et al. reported variant KGF proteins having heparin binding activity even after deleting 3, 8, or 27 amino-terminal amino acid residues (J. Biol. Chem. 268: 2984-2988 (1993)).

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N— or C-terminal residues of a protein retains such immunogenic activities can readily be determined by routine methods described herein and otherwise known in the art.

Thus, the invention further includes polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie et al., Science, 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, the amino acid positions which have been conserved between species can be identified. These conserved amino acids are likely important for protein function. In contrast, the amino acid positions in which substitutions have been tolerated by natural selection indicate positions which are not critical for protein function. Thus, positions tolerating amino acid substitution may be modified while still maintaining biological activity of the protein.

The second strategy uses genetic engineering to introduce amino acid changes at specific positions of a cloned gene to identify regions critical for protein function. For example, site-directed mutagenesis or alanine-scanning mutagenesis (the introduction of single alanine mutations at every residue in the molecule) can be used (Cunningham et al., Science, 244:1081-1085 (1989)). The resulting mutant molecules can then be tested for biological activity.

According to Bowie et al., these two strategies have revealed that proteins are surprisingly tolerant of amino acid substitutions. The authors further indicate which amino acid changes are likely to be permissive at certain amino acid positions in the protein. For example, the most buried or interior (within the tertiary structure of the protein) amino acid residues require nonpolar side chains, whereas few features of surface or exterior side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln; replacement of the basic residues Lys, Arg, and His; replacement of the aromatic residues Phe, Tyr, and Trp; and replacement of the small-sized amino acids Ala, Ser, Thr, Met, and Gly.

Besides conservative amino acid substitution, variants of the present invention include: (i) substitutions with one or more of the non-conserved amino acid residues, where the substituted amino acid residues may or may not be one encoded by the genetic code; (ii) substitution with one or more of amino acid residues having a substituent group; (iii) fusion of the mature polypeptide with another compound, such as a compound to increase the stability and/or solubility of the polypeptide (e.g., polyethylene glycol); (iv) fusion of the polypeptide with additional amino acids, such as an IgG Fc fusion region peptide, a leader or secretory sequence, or a sequence facilitating purification. Such variant polypeptides are deemed to be within the scope of those skilled in the art from the teachings herein.

Polynucleotide and Polypeptide Fragments

In the present invention, a “polynucleotide fragment” refers to a short polynucleotide having a nucleic acid sequence contained in that shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. The short nucleotide fragments are preferably at least about 15 nucleotides (nt), and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in that shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. These nucleotide fragments are useful as diagnostic probes and primers as discussed herein. Of course, larger fragments (e.g., 50, 150, and greater than 150 nucleotides) are preferred.

Moreover, representative examples of polynucleotide fragments of the invention, include, for example, fragments having a sequence from about nucleotide number 1-50, 51-100, 101-150, 151-200, 201-250, 251-300, 301-350, 351-400, 401-450, to the end of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. In this context “about” includes the particularly recited ranges, larger or smaller by several nucleotides (i.e., 5, 4, 3, 2, or 1 nt) at either terminus or at both termini. Preferably, these fragments encode a polypeptide which has biological activity.

In the present invention, a “polypeptide fragment” refers to a short amino acid sequence contained in the translations of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ-ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Protein fragments may be “free-standing,” or comprised within a larger polypeptide of which the fragment forms a part or region, most preferably as a single continuous region. Representative examples of polypeptide fragments of the invention include, for example, fragments from about amino acid number 1-20, 21-40, 41-60, or 61 to the end of the coding region. Moreover, polypeptide fragments can be about 20, 30, 40, 50, or 60 amino acids in length. In this context “about” includes the particularly recited ranges, larger or smaller by several amino acids (5, 4, 3, 2, or 1) at either extreme or at both extremes.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids ranging from 1-60, can be deleted from the amino terminus of either the secreted polypeptide or the mature form. Similarly, any number of amino acids ranging from 1-30, can be deleted from the carboxy terminus of the secreted protein or mature form. Furthermore, any combination of the above amino and carboxy terminus deletions are preferred. Similarly, polynucleotide fragments encoding these polypeptide fragments are also preferred.

Also preferred are polypeptide and polynucleotide fragments characterized by structural or functional domains, such as fragments that comprise alpha-helix and alpha-helix-forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of the translations of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 falling within conserved domains are specifically contemplated by the present invention. Moreover, polynucleotide fragments encoding these domains are also contemplated.

Other preferred fragments are biologically active fragments Biologically active fragments are those exhibiting activity similar, but not necessarily identical, to an activity of the polypeptide of the present invention. The biological activity of the fragments may include an improved desired activity, or a decreased undesirable activity.

Epitopes & Antibodies

Fragments which function as epitopes may be produced by any conventional means. (See, e.g., Houghten, R. A., Proc. Natl. Acad. Sci. USA, 82:5131-5135 (1985), further described in U.S. Pat. No.4,631,211.

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, e.g., Wilson et al., Cell, 37:767-778 (1984); Sutcliffe et al., Science, 219:660-666 (1983)).

Similarly, immunogenic epitopes can be used to induce antibodies according to methods well known in the art. (See, e.g., Sutcliffe et al., (1983) Supra; Wilson et al., (1984) Supra; Chow et al., Proc. Natl. Acad. Sci., USA, 82:910-914; and Bittle et al., J. Gen. Virol., 66:2347-2354 (1985)). A preferred immunogenic epitope includes the secreted protein. The immunogenic epitope may be presented together with a carrier protein, such as an albumin, to an animal system (such as rabbit or mouse). Alternatively, the immunogenic epitope may be prescribed without a carrier, if the sequence is of sufficient length (at least about 25 amino acids). However, immunogenic epitopes comprising as few as 8 to 10 amino acids have been shown to be sufficient to raise antibodies capable of binding to, at the very least, linear epitopes in a denatured polypeptide (e.g., in Western blotting.) As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody (Wahl et al;, J. Nucl. Med., 24:316-325 (1983)). Thus, these fragments are preferred, as well as the products of a Fab or other immunoglobulin expression library. Moreover, antibodies of the present invention include chimeric, single chain, and human and humanized antibodies.

The antibodies may be chimeric antibodies, e.g., humanized versions of murine monoclonal antibodies. Such humanized antibodies may be prepared by known techniques, and offer the advantage of reduced immunogenicity when the antibodies are administered to humans. Co et al. (Nature, 351:501-2, 1991). In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature, 332:323, 1988), Liu et al. (PNAS, 84:3439, 1987), Larrick et al. (Bio/Technology, 7:934, 1989), and Winter and Harris (TIPS, 14:139, May, 1993), Zou et al. (Science 262:1271-4, 1993), Zou et al. (Curr. Biol., 4:1099-103, 1994) and Walls et al. (Nucleic Acids Res., 21:2921-9, 1993).

One method for producing a human antibody comprises immunizing a non-human animal, such as a transgenic mouse, with a polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80, whereby antibodies directed against the polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 are generated in said animal. Procedures have been developed for generating human antibodies in non-human animals. The antibodies may be partially human, or preferably completely human. For example, mice have been prepared in which one or more endogenous immunoglobulin genes are inactivated by various means and human immunoglobulin genes are introduced into the mice to replace the inactivated mouse genes. Such transgenic mice may be′ genetically altered in a variety of ways. The genetic manipulation may result in human immunoglobulin polypeptide chains replacing endogenous immunoglobulin chains in at least some (preferably virtually all) antibodies produced by the animal upon immunization. Examples of techniques for production and use of such transgenic animals are described in U.S. Pat. Nos. 5,814,318, 5,569,825, and 5,545,806, which are incorporated by reference herein. Antibodies produced by immunizing transgenic animals with a polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 are provided herein.

Monoclonal antibodies may be produced by conventional procedures, e.g., by immortalizing spleen cells harvested from the transgenic animal after completion of the immunization schedule. The spleen cells may be fused with myeloma cells to produce hybridomas by conventional procedures. Examples of such techniques are described in U.S. Pat. No. 4,196,265, which is incorporated by reference herein.

A method for producing a hybridoma cell line comprises immunizing such a transgenic animal with an immunogen comprising at least seven contiguous amino acid residues of a polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80; harvesting spleen cells from the immunized animal; fusing the harvested spleen cells to a myeloma cell line, thereby generating hybridoma cells; and identifying a hybridoma cell line that produces a monoclonal antibody that binds a polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Such hybridoma cell lines, and monoclonal antibodies produced therefrom, are encompassed by the present invention. Monoclonal antibodies secreted by the hybridoma cell line are purified by conventional techniques. Examples of such techniques are described in U.S. Pat. No. 4,469,630 and U.S. Pat. No. 4,361,549.

Antibodies may be employed in an in vitro procedure, or administered in vivo to inhibit biological activity induced by a polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO: 16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Disorders caused or exacerbated (directly or indirectly) by the interaction of such polypeptides of the present invention with cell surface receptors thus may be treated. For example, chronic administration-of neuroleptics can cause unwanted side effects. Administration of an antibody derived from the identified polynucleotides might block the signaling that causes these side effects. Alternatively, an antibody derived from the identified polynucleotides might selectively block proteins causing motor side effects. A therapeutic method involves ii? vivo administration of a blocking antibody to a mammal in an amount effective for reducing a biological activity induced by a polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO: 19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. For example, chronic administration of neuroleptics can cause unwanted side effects. Administration of an antibody derived from the translation sequence of identified polynucleotides might block the signaling that causes these side effects. Alternatively, an antibody derived from the translation sequence of identified polynucleotides might selectively block proteins causing motor side effects.

Also provided herein are conjugates comprising a detectable (e.g., diagnostic) or therapeutic agent, attached to an antibody directed against a polypeptide translated from a nucleotide sequence chosen from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Examples of such agents are well known, and include but are not limited to diagnostic radionuclides, therapeutic radionuclides, and cytotoxic drugs. See, e.g., Thrush et. al (Annu. Rev. Immmunol., 14:49-71, 1996, p. 41). The conjugates find use in in vitro or in vivo procedures.

Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides are familiar and routine techniques in the art.

In addition, polypeptides of the present invention, including fragments and, specifically, epitopes, can be combined with parts of the constant domain of immunoglobulins (IgG), resulting in chimeric polypeptides. These fusion proteins facilitate purification and show an increased half-life iii vivo. One reported example describes chimeric proteins consisting of the first two domains of the human CD4-polypeptide and various domains of the constant regions of the heavy or light chains of mammalian immunoglobulins (EP A 394,827; Traunecker et al., Nature, 331:84-86 (1988).) Fusion proteins having disulfide-linked dimeric structures (due to the IgG) can also be more efficient in binding and neutralizing other molecules, than the monomeric secreted protein or protein fragment alone (Fountoulakis et al., J. Biochem., 270:3958-3964 (1995)).

Similarly, EP A 0 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties (see, e.g., EP A 0 232 262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5 (See, Bennett et al., J. Mol. Recognition 8:52-58 (1995); Johanson et al., J. Biol. Chem., 270:9459-9471 (1995)).

Moreover, the polypeptides of the present invention can be fused to marker sequences, such as a peptide Which facilitates purification of the fused polypeptide. In preferred embodiments, the marker amino acid sequence is a hexa-histidine peptide, such as the tag provided in a pQE vector (QIAGEN, Inc., Chatsworth, Calif.), among others, many of which are commercially available. As described in Gentz et al., for instance, hexa-histidine provides for convenient purification of the fusion protein (Proc. Natl. Acad. Sci. USA 86:821-824 (1989)). Another peptide tag useful for purification, the “HA” tag, corresponds to an epitope derived from the influenza hemagglutinin protein (Wilson et al., Cell, 37:767 (1984)). Other fusion proteins may use the ability of the polypeptides of the present invention to target the delivery of a biologically active peptide. This might include focused delivery of a toxin to tumor cells, or a growth factor to stem cells.

Thus, any of these above fusions can be engineered using the polynucleotides or the polypeptides of the present invention. See, e.g., Curr. Prot. Mol. Bio., Chapter 9.6.

Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells. See, e.g., Curr. Prot. Mol. Bio., Chapters 9.9, 16.15.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells, and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, PNH16A, PNH-18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may, in fact, be expressed by a host cell lacking a recombinant vector.

A polypeptide of this invention can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography (“HPLC”) is employed for purification.

Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Polypeptides of the present invention, and preferably the secreted form, can also be recovered from products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells.

Diagnosis and Treatment

Where a polynucleotdie of the invention is up-regulated, such as after chronic treatment with clozapine, the expression of the polynucleotide can be increased or the level of the intact polypeptide product can be increased in order to treat, prevent, ameliorate, or modulate the pathological condition. For example, increased expression of the SEQ ID NO:# (CLZ_{—}5) was observed after chronic treatment with clozapine. By increasing in vivo levels of such polynucleotides or polypeptide products, it may be possible to inhibit symptoms or reduce the severity of symptoms of schizophrenia or other neuropsychiatric disorders. This can be accomplished by, for example, administering a polynucleotide or polypeptide of the invention (or a set of polynucleotides and polypeptides including those of the invention) to the mammalian subject.

A polynucleotide of the invention can be administered alone or withother polynucleotides to a mammalian subject by a recombinant expression vector comprising the polynucleotide. A mammalian subject can be a human, baboon, chimpanzee, macaque, cow, horse, sheep, pig, horse, dog, cat, rabbit, guinea pig, rat or mouse. Preferably, the recombinant vector comprises a polynucleotide shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 or a polynucleotide which is at least 98% identical to a nucleic acid sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Also, preferably, the recombinant vector comprises a variant polynucleotide that is at least 80%, 90%, or 95% identical to a polynucleotide comprising SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80.

The administration of a polynucleotide or recombinant expression vector of the invention to a mammalian subject can be used to express a polynucleotide in said subject for the treatment of neurological and psychiatric disorders, for example, schizophrenia. Expression of a polynucleotide in target cells, including but not limited to brain cells, would effect greater production of the encoded polypeptide. In some cases, where the encoded polypeptide is a nuclear protein, the regulation of other genes may be secondarily up- or down-regulated. The administration of a polynucleotide or recombinant expression vector of the invention to a mammalian subject can be used to express a polynucleotide in the said subject for the treatment of, for example, psychosis or other neuropsychiatric disorders. Expression of a polynucleotide in target cells, including but not limited to the cells of the striatum and nucleus accumbens, would effect greater production of the encoded polypeptide. High expression of the polynucleotide would be advantageous since increased expression was observed after chronic treatment with clozapine.

There are available to one skilled in the art multiple viral and non-viral methods suitable for introduction of a nucleic acid molecule into a target cell, as described above. In addition, a naked polynucleotide can be administered to target cells. Polynucleotides and recombinant expression vectors of the invention can be administered as a pharmaceutical composition. Such a composition comprises an effective amount of a polynucleotide or recombinant expression vector, and a pharmaceutically acceptable formulation agent selected for suitability with the mode of administration. Suitable formulation materials preferably are non-toxic to recipients at the concentrations employed and can modify, maintain, or preserve, for example, the pH, osmolarity, viscosity, clarity, color, isotonicity, odor, sterility, stability, rate of dissolution or release, adsorption, or penetration of the composition. See Remington's Pharmaceutical Sciences (18th Ed., A. R. Gennaro, ed., Mack Publishing Company 1990).

The pharmaceutically active compounds (i.e., a polynucleotide or a vector) can be processed in accordance with conventional methods of pharmacy to produce medicinal agents for administration to patients, including humans and other mammals. Thus, the pharmaceutical composition comprising a polynucleotide or a recombinant expression vector may be made up in a solid form (including granules, powders or suppositories) or in a liquid form (e.g., solutions, suspensions, or emulsions).

The dosage regimen for treating a disease with a composition comprising a polynucleotide or expression vector is based on a variety of factors, including the type or severity of the neurological or psychiatric disorder the age, weight, sex, medical condition of the patient, the route of administration, and the particular compound employed. Thus, the dosage regimen may vary widely, but can be determined routinely using standard methods. A typical dosage may range from about 0.1 mg/kg to about 100 mg/kg or more, depending on the factors mentioned above.

The frequency of dosing will depend upon the pharmacokinetic parameters of the polynucleotide or vector in the formulation being used. Typically, a clinician will administer the composition until a dosage is reached that achieves the desired effect. The composition may therefore be administered as a single dose, as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data.

The cells of a mammalian subject may be transfected in vivo, ex vivo, or in vitro. Administration of a polynucleotide or a recombinant vector containing a polynucleotide to a target cell in vivo may be accomplished using any of a variety of techniques well known to those skilled in the art. For example, U.S. Pat. No. 5,672,344 describes an in vivo viral-mediated gene transfer system involving a recombinant neurotrophic HSV-1 vector. The above-described compositions of polynucleotides and recombinant vectors can be transfected in vivo by oral, buccal, parenteral, rectal, or topical administration as well as by inhalation spray. The term “parenteral” as used herein includes, subcutaneous, intravenous, intramuscular, intrasternal, infusion techniques or intraperitoneally.

While the nucleic acids and/or vectors of the invention can be administered as the sole active pharmaceutical agent, they can also be used in combination with one or more vectors of the invention or other agents. When administered as a combination, the therapeutic agents can be formulated as separate compositions that are given at the same time or different times, or the therapeutic agents can be given as a single composition.

Another delivery system for polynucleotides of the invention is a “non-viral” delivery system. Techniques that have been used or proposed for gene therapy include DNA-ligand complexes, adenovirus-ligand-DNA complexes, direct injection of DNA, CaPO4 precipitation, gene gun techniques, electroporation, lipofection, and colloidal dispersion (Mulligan, R., (1993) Science, 260 (5110):926-32). Any of these methods are widely available to one skilled in the art and would be suitable for use in the present invention. Other suitable methods are available to one skilled in the art, and it is to be understood that the present invention may be accomplished using any of the available methods of transfection. Several such methodologies have been utilized by those skilled in the art with varying success (Mulligan, R., (1993) Science, 260 (5110):926-32).

Where a polynucleotide of the invention is down-regulated, such as after chronic treatment with clozapine, the expression of the polynucleotide can be blocked or reduced or the level of the intact polypeptide product can be reduced in order to treat, prevent, ameliorate, or modulate the pathological condition, such as psychosis or other neuropsychiatric disorders. For example, decreased expression of the polynucleotides with the SEQ ID NOs: 15, 28, 29, and 12 (CLZ_{—}16, CLZ_{—}17, CLZ_{—}26, and CLZ_{—}40), were down-regulated after chronic-administration of clozapine. This activity may represent pathways common to the beneficial effects of clozapine treatment of psychosis or other neuropsychiatric disorders. By decreasing the in vivo levels of such polynucleotides or polypeptide products, it may be possible to inhibit symptoms or reduce the severity of symptoms of schizophrenia or other neuropsychiatric disorders. This can be accomplished by, for example, the use of antisense oligonucleotides, triple helix base pairing methodology or ribozymes. Alternatively, drugs or antibodies that bind to and inactivate the polypeptide product can be used.

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of gene products of the invention in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, (1994) Meth. Mol. Biol., 20:1-8; Sonveaux, (1994) Meth. Mol. Biol., 26:1-72; Uhlmann et al., (1990) Chem. Rev., 90:543-583.

Modifications of gene expression can be obtained by designing antisense oligonucleotides which will form duplexes to the control, 5′, or regulatory regions of a gene of the invention. Oligonucleotides derived from the transcription initiation site, e.g., between positions −10 and +10 from the start site, are preferred.

Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a polynucleotide. Antisense oligonucleotides which comprise, for example, 2, 3, 4, or 5 or more stretches of contiguous nucleotides which are precisely complementary to a polynucleotide, each separated by a stretch of contiguous nucleotides which are not complementary to adjacent nucleotides, can provide sufficient targeting specificity for mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular polynucleotide sequence.

Antisense oligonucleotides can be modified without affecting their ability to hybridize to a polynucleotide of the invention. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., (1992) Trends Biotechnol., 10:152-158; Uhlmann et al., (1990) Chem. Rev., 90:543-584; Uhlmann et al., (1987) Tetrahedron. Lett., 215:3539-3542.

Ribozymes are RNA molecules with catalytic activity. See, e.g., Cech, (1987) Science, 236:1532-1539; Cech, (1990) Ann. Rev. Biochem., 59:543-568; Cech, (1992) Curr. Opin. Struct. Biol., 2:605-609; Couture & Stinchcomb, (1996) Trends Genet., 12:510-515. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a polynucleotide of the invention can be used to generate ribozymes which will specifically bind to mRNA transcribed from the polynucleotide. Methods of designing and constructing ribozymes which can cleave RNA molecules-in-trans in a highly sequence specific manner have been developed and described in the art (see Haseloff et al. (1988) Nature, 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, e.g., Gerlach et al., EP 321,201).

Specific ribozyme cleavage sites within a RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. The nucleotide sequences shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 and their complements provide sources of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease polynucleotide expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Production of Diagnostic Tests

Pathological conditions or susceptibility to pathological conditions, such as psychoses or other neuropsychiatric disorders, can be diagnosed using methods of the invention. Testing for expression of a polynucleotide of the invention or for the presence of the polynucleotide product can correlate with the severity of the condition and can also indicate appropriate treatment. For example, the presence or absence of a mutation in a polynucleotide of the invention can be determined and a pathological condition or a susceptibility to a pathological condition is diagnosed based on the presence or absence of the mutation. Further, an alteration in expression of a polypeptide encoded by a polynucleotide of the invention can be detected, where the presence of an alteration in expression of the polypeptide is indicative of the pathological condition or susceptibility to the pathological condition. The alteration in expression can be an increase in the amount of expression or a decrease in the amount of expression.

As an additional method of diagnosis, a first biological sample from a patient suspected of having a pathological condition, such as psychoses or other neuropsychiatric disorders, is obtained along with a second sample from a suitable comparable control source. A biological sample can comprise saliva, blood, cerebrospinal fluid, amniotic fluid, urine, feces, or tissue, such as gastrointestinal tissue. A suitable control source can be obtained from one or more mammalian subjects that do not have the pathological condition. For example, the average concentrations and distribution of a polynucleotide or polypeptide of the invention can be determined from biological samples taken from a representative population of mammalian subjects, wherein the mammalian subjects are the same species as the subject from which the test sample was obtained. The amount of at least one polypeptide encoded by a polynucleotide of the invention is determined in the first and second sample. The amounts of the polypeptide in the first and second samples are compared. A patient is diagnosed as having a pathological condition if the amount of the polypeptide in the first sample is greater than or less than the amount of the polypeptide in the second sample. Preferably, the amount of polypeptide in the first sample falls within the range of samples taken from a representative group of patients with the pathological condition.

The method for diagnosing a pathological condition can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

The present invention also includes a diagnostic system, preferably in kit form, for assaying for the presence of the polypeptide of the present invention in a body sample, such as brain tissue, cell suspensions or tissue sections; or a body fluid sample, such as CSF, blood, plasma or serum, where it is desirable to detect the presence, and preferably the amount, of the polypeptide of this invention in the sample according to the diagnostic methods described herein.

In a related embodiment, a nucleic acid molecule can be used as a probe (i.e., an oligonucleotide) to detect the presence of a polynucleotide of the present invention, a gene corresponding to a polynucleotide of the present invention, or a mRNA in a cell that is diagnostic for the presence or expression of a polypeptide of the present invention in the cell. The nucleic acid molecule probes can be of a variety of lengths from at least about 10, suitably about 10 to about 5000 nucleotides long, although they will typically be about 20 to 500 nucleotides in length. The probe can be used to detect the polynucleotide through] hybridization methods which are extremely well known in the art and will not be described further here.

In a related embodiment, detection of genes corresponding to the polynucleotides of the present invention can be conducted by primer extension reactions such as the polymerase chain reaction (PCR). To that end, PCR primers are utilized in pairs, as is well known, based on the nucleotide sequence of the gene to be detected. Preferably, the nucleotide sequence is a portion of the nucleotide sequence of a polynucleotide of the present invention. Particularly preferred PCR primers can be derived from any portion of a DNA sequence encoding a polypeptide of the present invention, but are preferentially from regions which are not conserved in other cellular proteins.

Preferred PCR primer pairs useful for detecting the genes corresponding to the polynucleotides of the present invention and expression of these genes are described in the TOGA™ Process Section above and in the Tables. Nucleotide primers from the corresponding region of the polypeptides of the present invention described herein are readily prepared and used as PCR primers for detection of the presence or expression of the corresponding gene in any of a variety of tissues.

In another embodiment, a diagnostic system, preferably in kit form, is contemplated for assaying for the presence of the polypeptide of the present invention or an antibody immunoreactive with the polypeptide of the present invention in a body fluid sample. Such diagnostic kit would be useful for monitoring the fate of a therapeutically administered polypeptide of the present invention or an antibody immunoreactive with the polypeptide of the present invention. The system includes, in an amount sufficient for at least one assay, a polypeptide of the present invention and/or a subject antibody as a separately packaged immunochemical reagent.

Instructions for use of the packaged reagent(s) are also typically included.

A diagnostic system of the present invention preferably also includes a label or indicating means capable of signaling the formation of an immunocomplex containing a polypeptide or antibody molecule of the present invention.

Any label or indicating means can be linked to or incorporated in an expressed protein, polypeptide, or antibody molecule that is part of an antibody or monoclonal antibody composition of the present invention or used separately, and those atoms or molecules can be used alone or in conjunction with additional reagents. Such labels are themselves well-known in clinical diagnostic chemistry and constitute a part of this invention only insofar as they are utilized with otherwise novel proteins methods and/or systems.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyanate (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like. A description of immunofluorescence analysis techniques is found in DeLuca, “Immunofluorescence Analysis”, in Antibody As a Tool, Marchalonis et al., Eds., John Wiley & Sons, Ltd., pp. 189-231 (1982), which is incorporated herein by reference. Other suitable labeling agents are known to those skilled in the art.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-amino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is 125I. Another group of useful labeling means are those elements such as ¹¹C, ¹⁸F, 15O and ¹³N which themselves emit positrons. The positrons so emitted produce gamma rays upon encounters with electrons present in the animal's body. Also useful is a beta emitter, such ¹¹¹indium or ³H.

The linking of labels or labeling of polypeptides and proteins is well known in the art. For instance, antibody molecules produced by a hybridoma can be labeled by metabolic incorporation of radioisotope-containing amino acids provided as a component in the culture medium (see, e.g., Galfre et al., Methl. Enzymol., 73:3-46 (1981)). The techniques of protein conjugation or coupling through activated functional groups are particularly applicable (see, e.g., Aurameas, et al., Scand. J. Immunol., Vol. 8 Suppl. 7:7-23 (1978); Rodwell et al., Biotech., 3:889-894 (1984); and U.S. Pat. No. 4,493,795).

The diagnostic systems can also include, preferably as a separate package, a specific binding agent. Exemplary specific binding agents are second antibody molecules, complement proteins or fragments thereof, S. aureus protein A, and the like. Preferably the specific binding agent binds the reagent species when that species is present as part of a complex.

In preferred embodiments, the specific binding agent is labeled. However, when the diagnostic system includes a specific binding agent that is not labeled, the agent is typically used as an amplifying means or reagent. In these embodiments, the labeled specific binding agent is capable of specifically binding the amplifying means when the amplifying means is bound to a reagent species-containing complex.

The diagnostic kits of the present invention can be used in an “ELISA” format to detect the quantity of the polypeptide of the present invention in a sample. A description of the ELISA technique is found in Sites et al., Basic and Clinical Immunology, 4th Ed., Chap. 22, Lange Medical Publications, Los Altos, Calif. (1982) and in U.S. Pat. No. 3,654,090; U.S. Pat. No. 3,850,752; and U.S. Pat. No. 4,016,043, which are all incorporated herein by reference.

Thus, in some embodiments, a polypeptide of the present invention, an antibody or a monoclonal antibody of the present invention can be affixed to a solid matrix to form a solid support that comprises a package in the subject diagnostic systems.

A reagent is typically affixed to a solid matrix by adsorption from an aqueous medium, although other modes of affixation applicable to proteins and polypeptides can be used that are well known to those skilled in the art. Exemplary adsorption methods are described herein.

Useful solid matrices are also well known in the art. Such materials are water insoluble and include the cross-linked dextran available under the trademark SEPHADEX from Pharmacia Fine Chemicals (Piscataway, N.J.), agarose, polystyrene beads of about 1 micron (μm) to about 5 millimeters (mm) in diameter available from several suppliers (e.g., Abbott Laboratories, Chicago, Ill.), polyvinyl chloride, polystyrene, cross-linked polyacrylamide, nitrocellulose- or nylon-based webs (sheets, strips or paddles) or tubes, plates or the wells of a microtiter plate, such as those made from polystyrene or polyvinylchloride.

The reagent species, labeled specific binding agent, or amplifying reagent of any diagnostic system described herein can be provided in solution, as a liquid dispersion or as a substantially dry power, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

The packaging materials discussed herein in relation to diagnostic systems are those customarily utilized in diagnostic systems.

Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents based on actual sequence data (repeat polymorphisms) are presently available. Each polynucleotide of the present invention can be used as a chromosome marker. Currently, no specific diagnostic markers exist that can be used to prevent or delay psychotic episodes of schizophrenia. The polynucleotides of the present invention may be used as chromosome markers for diagnosis for schizophrenia.

Briefly, sequences can be mapped to chromosomes by preparing PCR primers (preferably 15-25 bp) from the sequences shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14,.SEQ ID NO:15, SEQ ID NO:16, SEQ ID NO:17, SEQ ID NO:18, SEQ ID NO:19,.SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene-mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides of 2,0004,000 bp are preferred. For a review of this technique, see Verma et al., Human Chromosomes: a Manual of Basic Techniques, Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross-hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. Disease mapping data are found, for example, in V. McKusick, Mendelian Inheritance in Man (available on line through Johns Hopkins University Welch Medical Library), Kruglyak et al. (Am. J. Hum. Genet., 56:1212-23, 1995); Curr. Prot. Hum. Genet. Assuming one megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected individuals can be examined. The polynucleotides of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 can be used for this analysis of individuals. As noted above, many psychiatric disorders have genetic etiology and using the polynucleotides of the present invenion in a diagnostic panel can facilitate in the diagnosis of patients or identify patients at risk.

First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using polynucleotides of the present invention. Any of these alterations (altered expression, chromosomal rearrangement, or mutation) can be used as a diagnostic or prognostic marker.

In addition to the foregoing, a polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (see, Lee et al., Nuc. Acids Res., 6:3073 (1979); Cooney et al., Science, 241:456 (1988); and Dervan et al., Science, 251:1360 (1991) for discussion of triple helix formation) or to the mRNA itself (see, Okano, J. Neurochem, 56:566 (1991); and Oligodeoxy-nucleotides as Antisense Inhibitors of Gene Expression, CRC Press, Boca-Raton, Fla. (1988) for a discussion of antisense technique). Triple helix formation optimally results in a shut-off of RNA transcription from DNA, while antisense RNA hybridization blocks translation of an. mRNA molecule into polypeptide. Both techniques are effective in model systems, and the information disclosed herein can be used to design antisense or triple helix polynucleotides in an effort to treat disease.

Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods (Jalkanen, et al., J. Cell. Biol., 101:976-985 (1985); Jalkanen, et al., J. Cell. Biol., 105:3087-3096 (1987)). Other antibody-based methods useful for detecting protein gene expression include immunoassays, such as the enzyme linked immunosorbent assay (ELISA) and the radioimmunoassay (RIA). See, e.g., Curr. Prot. Mol. Bio., Chapter 11. Suitable antibody assay labels are known in the art and include enzyme labels, such as glucose oxidase; and radioisotopes, such as iodine (¹²⁵I, ¹²¹I), carbon (¹⁴C), sulfur (³⁵S), tritium (³H), indium (¹¹¹In), and technetium (^{99m}Tc); fluorescent labels, such as fluorescein and rhodamine; and biotin.

In addition to assaying secreted protein levels in a biological sample, proteins can also be detected in vivo by imaging. Antibody labels or markers for in vivo imaging of protein include those detectable by X-radiography, nuclear magnetic resonance (NMR), or electron spin resonance (ESR). For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety such as a radioisotope (e.g., ¹³¹I, ¹¹¹In, ^{99m}Tc), a radio-opaque substance, or a material detectable by NA, is introduced (e.g., parenterally, subcutaneously, or intraperitoneally) into the mammal. It will be understood in the art that the size of the subject and the imaging system used will determine the quantity of imaging moiety needed to produce diagnostic images. In the case of a radioisotope moiety, the quantity of radioactivity necessary for a human subject will normally range from about 5 to 20 millicuries of ^{99m}Tc. The labeled antibody or antibody fragment will then preferentially accumulate at the location of cells which contain the specific protein. In vivo tumor imaging is described in Burchiel et al., “Immunopharmacokinetics of Radiolabeled Antibodies and Their Fragments” (Chapter 13 in Tumor Imaging: The Radiochemical Detection of Cancer, Burchiel and Rhodes, Eds., Masson Publishing Inc. (1982)).

Thus, the invention provides a diagnostic method of a disorder, which involves (a) assaying the expression of a polypeptide of the present invention in cells or body fluid of an individual; and (b) comparing the level of gene expression with a standard gene expression level, whereby an increase or decrease in the assayed polypeptide gene expression level compared to the standard expression level is indicative of a disorder. Psychiatric disorders and treatment of psychiatric disorders with neuroleptics, including schizophrenia, are associated with a dysregulation of neurotransmitter and/or neuropeptide levels that can result in the up- or down regulation of polynucleotides and polypeptides. These changes can be diagnosed or monitored by assaying changes in polypeptide levels in tissue or fluids such as CSF, blook, or in fecal samples.

Moreover, polypeptides of the present invention can be used to treat disease. For example, schizophrenic patients can be administered a polypeptide of the present invention in an effort to replace absent or decreased levels of the polypeptide; to supplement absent or decreased levels of a different polypeptide (e.g., hemoglobin S for hemoglobin B); to inhibit the activity of a polypeptide (e.g., an oncogene); to activate the activity of a polypeptide (e.g., by binding to a receptor); to reduce the activity of a membrane bound receptor by competing with it for free ligand (e.g., soluble tumor necrosis factor (TNF) receptors used in reducing inflammation); or to bring about a desired response (e.g., blood vessel growth).

Similarly, antibodies directed to a polypeptide of the present invention can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor). Polypeptides can be used as antigens to trigger immune responses. Local production of neurotransmitters and neuropeptides modulates many aspects of neuronal function. For example, in schizophrenia overactive neurotransmitter activity is thought to be basis for the psychotic behavior. Administration of an antibody to an overproduced polypeptide can be used to modulate neuronal responses in psychiatric disorders such as schizophrenia.

Polypeptides can also be used to raise antibodies, which in turn are used to measure protein expression from a recombinant cell, as a way of assessing transformation of the host cell. See, e.g., Curr. Prot. Mol. Bio., Chapter 11.15. Moreover, the polypeptides of the present invention can be used to test the following biological activities.

Biological Activities

The polynucleotides and polypeptides of the present invention can be used in assays to test for one or more biological activities. If these polynucleotides and polypeptides do exhibit activity in a particular assay, it is likely that these molecules may be involved in the diseases associated with the biological activity. Thus, the polynucleotides and polypeptides could be used to treat the associated disease.

Nervous System Activity

A polypeptide or polynucleotide of the present invention may be useful in treating deficiencies or disorders of the central nervous system or peripheral nervous system by activating or inhibiting the proliferation, differentiation, or mobilization (chemotaxis) of neuroblasts, stem cells, or glial cells. Also, a polypeptide or polynucleotide of the present invention may be useful in treating deficiencies or disorders of the central nervous system or peripheral nervous system by activating or inhibiting the mechanisms of synaptic transmission, synthesis, metabolism and inactivation of neural transmitters, neuromodulators and trophic factors, and by activating or inhibiting the expression and incorporation of enzymes, structural proteins, membrane channels, receptors in neurons and glial cells, or altering neural membrane compositions.

The etiology of these deficiencies or disorders may be genetic, somatic (such as cancer or some autoimmune disorder), acquired (e.g., by chemotherapy or toxins), or infectious. Moreover, a polynucleotide or polypeptide of the present invention can be used as a marker or detector of a particular nervous system disease or disorder. The disorder or disease can be any of Alzheimer's disease, Pick's disease, Binswanger's disease, other senile dementia, Parkinson's disease, parkinsonism, obsessive compulsive disorders, epilepsy, encephaolopathy, ischemia, alcohol addiction, drug addiction, schizophrenia, amyotrophic lateral sclerosis, multiple sclerosis, depression, and bipolar manic-depressive disorder. Alternatively, the polypeptide or polynucleotide of the present invention can be used to study circadian variation, aging, or long-term potentiation, the latter affecting the hippocampus. Additionally, particularly with reference to mRNA species occurring in particular structures within the central nervous system, the polypeptide or polynucleotide of the present invention can be used to study brain regions that are known to be involved in complex behaviors, such as learning and memory, emotion, drug addiction, glutamate neurotoxicity, feeding behavior, olfaction, viral infection, vision, and movement disorders.

Binding Activity

A polypeptide of the present invention may be used to screen for molecules that bind to the polypeptide or for molecules to which the polypeptide binds. The binding of the polypeptide and the molecule may activate (i.e., an agonist), increase, inhibit (i.e., an antagonist), or decrease activity of the polypeptide or the molecule bound. Examples of such molecules include antibodies, oligonucleotides, proteins (e.g., receptors), or small molecules.

Preferably, the molecule is closely related to the natural ligand of the polypeptide, e.g., a fragment of the ligand, or a natural substrate, a ligand, a structural or functional mimetic (see, Coligan et al., Current Protocols in Immunology 1(2), Chapter 5 (1991)). Similarly, the molecule can be closely related to the natural receptor to which the polypeptide binds or, at least, related to a fragment of the receptor capable of being bound by the polypeptide (e.g., an active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

All of these above assays can be used as diagnostic or prognostic markers. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues. At present, diagnosis of schizophrenia is based on clinical assessment and not on any lab test. Thus, the availability of an objective laboratory diagnostic will be of great value in the diagnosis and assessment of patients through treatment regimens.

Therefore, the invention includes a method of identifying compounds which bind to a polypeptide of the invention comprising the steps of. (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity, and (c) determining if a-biological activity of the polypeptide has been altered.

Other Activities

A polypeptide or polynucleotide of the present invention may also increase or decrease the differentiation or proliferation of embryonic stem cells from a lineage other than the above-described hemopoietic lineage. A polypeptide or polynucleotide of the present invention may also increase or decrease the differentiation or proliferation of embryonic stem cells from a lineage other than the above-described hemopoietic lineage. Expression of a polynucleotide or polypeptide of the present invention may be associated with various types of CNS pathology, including psychosis or other neuropsychiatric disorders. Specifically, SEQ ID NO: 2 (CLZ_{—}5) expression has been associated not only been associate with clozapine treatment, and schizophrenic and bipolar patients, it has also been associated with specific brain regions of a mouse model for Alzheimer's disease. Alzheimer's disease is an example of a disease that is accompanied by degenerating neuronal cells. Repopulation of lost neurons would be a feasible treatment option if molecules existed to promote the differentiation.

A polypeptide or polynucleotide of the present invention may also be used to modulate mammalian characteristics, such as body height, weight, hair color, eye color, skin, percentage of adipose tissue, pigmentation, size, and shape (e.g., cosmetic surgery). Similarly, a polypeptide or polynucleotide of the present invention may be used to modulate mammalian metabolism affecting catabolism, anabolism, processing, utilization, and storage of energy.

A polypeptide or polynucleotide of the present invention may be used to change a mammal's mental state or physical state by influencing biorhythms, circadian rhythms, depression (including depressive disorders), tendency for violence, tolerance for pain, the response to opiates and opioids, tolerance to opiates and opioids, withdrawal from opiates and opioids, reproductive capabilities (preferably by activin or inhibin-like activity), hormonal or endocrine levels, appetite, libido, memory, stress, or other cognitive qualities.

A polypeptide or polynucleotide of the present invention may also be used as a food additive or preservative, such as to increase or decrease storage capabilities, fat content, lipid, protein, carbohydrate, vitamins, minerals, cofactors, or other nutritional components.

The plynucleotides, polypeptides, kits and methods of the present invention may be embodied in other specific forms without departing from the teachings or essential characteristics of the invention. The described embodiments are therefore to be considered in all respects as illustrative and not restrictive, the scope of the invention being indicated by the appended claims rather than by the foregoing description, and all changes which come within the meaning and range of equivalency of the claims are therefore to be embraced therein.

TABLE 1

| Seq ID | | Digital Address | | | 45 | | | | |
|---|---|---|---|---|---|---|---|---|---|
| No | DST ID | (Mspl) | | Control | minutes | 7 Hour | 5 Day | 12 Day | 14 Day |
| | | AAAA | 276 | 314 | 189 | 183 | 299 | 292 | 227 |
| | | AAAG | 91 | 18 | 27 | 34 | 52 | 60 | 35 |
| | | AAAG | 446 | 135 | 127 | 219 | 245 | 529 | 210 |
| | | AAAG | 449 | 135 | 173 | 219 | 245 | 775 | 210 |
| | | AACA | 109 | 31 | 17 | 85 | 54 | 51 | 72 |
| | | AACA | 117 | 38 | 30 | 45 | 39 | 72 | 118 |
| | | AACA | 137 | 355 | 205 | 163 | 129 | 111 | 186 |
| | | AACA | 307 | 56 | 58 | 65 | 55 | 134 | 64 |
| | | AACG | 375 | 633 | 450 | 420 | 528 | 968 | 1015 |
| | | AACG | 498 | 717 | 221 | 349 | 438 | 1647 | 1392 |
| | | AACT | 85 | 481 | 139 | 145 | 108 | 281 | 580 |
| | | AACT | 112 | 297 | 162 | 391 | 538 | 330 | 555 |
| | | AACT | 392 | 176 | 267 | 427 | 303 | 296 | 315 |
| | | AAGA | 309 | 22 | 19 | 42 | 91 | 61 | 36 |
| | | AAGA | 324 | 37 | 15 | 12 | 83 | 31 | 46 |
| | | AAGC | 446 | 284 | 212 | 155 | 249 | 318 | 338 |
| | | AAGC | 498 | 456 | 369 | 309 | 495 | 735 | 862 |
| | | AAGG | 270 | 169 | 191 | 176 | 243 | 283 | 265 |
| | | AAGG | 457 | 191 | 129 | 152 | 228 | 269 | 320 |
| | | AAGG | 497 | 265 | 164 | 208 | 432 | 390 | 512 |
| | | AAGT | 282 | 75 | 73 | 82 | 84 | 204 | 105 |
| | | AATA | 90 | 47 | 46 | 39 | 74 | 115 | 65 |
| 39 | CLZ_47 | AATA | 136 | 817 | 555 | 589 | 297 | 245 | 397 |
| | | AATA | 194 | 70 | 81 | 70 | 133 | 181 | 112 |
| | | AATC | 352 | 108 | 108 | 128 | 144 | 631 | 140 |
| | | AATC | 499 | 49 | 32 | 43 | 67 | 75 | 67 |
| | | AATT | 425 | 38 | 30 | 38 | 37 | 64 | 45 |
| | | ACAA | 80 | 92 | 67 | 109 | 319 | 353 | 110 |
| | | ACAA | 122 | 58 | 95 | 107 | 46 | 818 | 98 |
| | | ACAA | 239 | 117 | 45 | 133 | 49 | 217 | 137 |
| | | ACAC | 145 | 313 | 365 | 296 | 277 | 750 | 631 |
| | | ACAC | 273 | 163 | 169 | 262 | 274 | 800 | 338 |
| | | ACAG | 81 | 167 | 81 | 57 | 137 | 314 | 253 |
| | | ACAG | 270 | 117 | 94 | 117 | 93 | 236 | 213 |
| | | ACAG | 296 | 32 | 34 | 71 | 47 | 89 | 62 |
| | | ACAG | 413 | 39 | 43 | 52 | 43 | 88 | 81 |
| | | ACAG | 437 | 25 | 20 | 41 | 22 | 55 | 41 |
| | | ACAT | 94 | 91 | 151 | 149 | 91 | 340 | 195 |
| | | ACCA | 109 | 318 | 505 | 352 | 289 | 189 | 200 |
| | | ACCA | 418 | 33 | 28 | 46 | 40 | 65 | 39 |
| | | ACCA | 422 | 28 | 23 | 44 | 39 | 51 | 39 |
| | | ACCC | 394 | 32 | 55 | 40 | 38 | 162 | 37 |
| | | ACCC | 493 | 54 | 42 | 57 | 48 | 93 | 69 |
| | | ACCG | 90 | 181 | 155 | 184 | 217 | 382 | 208 |
| | | ACCG | 220 | 169 | 113 | 262 | 189 | 335 | 247 |
| | | ACCG | 489 | 33 | 30 | 28 | 44 | 63 | 41 |
| | | ACCT | 119 | 117 | 121 | 47 | 86 | 300 | 164 |
| | | ACCT | 490 | 78 | 76 | 57 | 120 | 165 | 133 |
| | | ACGA | 77 | 567 | 133 | 109 | 72 | 1143 | 1079 |
| | | ACGA | 92 | 61 | 56 | 56 | 76 | 195 | 63 |
| | | ACGA | 292 | 349 | 247 | 165 | 190 | 306 | 148 |
| | | ACGC | 78 | 243 | 31 | 51 | 236 | 2323 | 1676 |
| | | ACGC | 118 | 1026 | 737 | 849 | 292 | 442 | 513 |
| | | ACGC | 210 | 243 | 284 | 293 | 343 | 682 | 735 |
| | | ACGC | 284 | 27 | 50 | 60 | 195 | 159 | 94 |
| | | ACGC | 474 | 50 | 91 | 87 | 107 | 190 | 131 |
| | | ACGG | 264 | 140 | 108 | 117 | 115 | 294 | 172 |
| | | ACGG | 335 | 245 | 104 | 102 | 110 | 131 | 159 |
| 38 | CLZ_44 | ACGG | 352 | 171 | 407 | 428 | 538 | 683 | 553 |
| | | ACGG | 382 | 37 | 53 | 113 | 154 | 141 | 103 |
| | | ACGG | 406 | 114 | 233 | 267 | 217 | 219 | 211 |
| | | ACTA | 88 | 28 | 37 | 33 | 29 | 219 | 41 |
| | | ACTA | 199 | 38 | 84 | 48 | 120 | 365 | 66 |
| | | ACTC | 88 | 64 | 30 | 71 | 124 | 108 | 81 |
| | | ACTC | 105 | 54 | 121 | 172 | 155 | 352 | 294 |
| | | ACTG | 266 | 23 | 35 | 116 | 35 | 87 | 44 |
| | | ACTG | 468 | 148 | 80 | 53 | 74 | 58 | 68 |
| | | ACTT | 436 | 490 | 549 | 450 | 494 | 435 | 504 |
| | | AGAA | 104 | 86 | 210 | 143 | 63 | 39 | 106 |
| | | AGAA | 196 | 62 | 75 | 43 | 85 | 172 | 97 |
| | | AGAA | 462 | 42 | 29 | 25 | 27 | 64 | 42 |
| | | AGAC | 410 | 362 | 307 | 538 | 530 | 918 | 442 |
| | | AGAT | 79 | 41 | 73 | 50 | 64 | 193 | 70 |
| | | AGAT | 251 | 622 | 622 | 746 | 691 | 562 | 696 |
| | | AGAT | 295 | 294 | 252 | 263 | 281 | 303 | 263 |
| | | AGAT | 456 | 603 | 525 | 571 | 639 | 588 | 559 |
| | | AGCA | 177 | 21 | 38 | 46 | 64 | 163 | 100 |
| | | AGCC | 295 | 661 | 444 | 517 | 421 | 360 | 475 |
| | | AGCC | 468 | 112 | 99 | 110 | 165 | 145 | 146 |
| | | AGCG | 202 | 385 | 349 | 433 | 339 | 334 | 334 |
| | | AGCT | 95 | 162 | 963 | 1168 | 2493 | 3990 | 1420 |
| | | AGCT | 260 | 89 | 78 | 58 | 296 | 86 | 294 |
| | | AGGA | 426 | 365 | 532 | 720 | 670 | 896 | 802 |
| | | AGGC | 104 | 46 | 86 | 169 | 163 | 642 | 339 |
| | | AGGG | 177 | 739 | 251 | 249 | 210 | 174 | 408 |
| | | AGGG | 242 | 165 | 110 | 192 | 222 | 376 | 293 |
| | | AGGG | 492 | 35 | 48 | 33 | 46 | 98 | 75 |
| | | AGGG | 498 | 50 | 47 | 69 | 79 | 155 | 111 |
| | | AGGT | 99 | 55 | 36 | 55 | 80 | 83 | 61 |
| | | AGGT | 103 | 29 | 27 | 31 | 50 | 84 | 38 |
| | | AGGT | 119 | 835 | 719 | 808 | 518 | 466 | 643 |
| 1 | CLZ_3 | AGTA | 106 | 657 | 1677 | 1883 | 894 | 832 | 1282 |
| | | AGTC | 97 | 297 | 229 | 215 | 158 | 111 | 180 |
| | | AGTC | 178 | 519 | 351 | 238 | 263 | 353 | 269 |
| | | AGTC | 410 | 65 | 93 | 107 | 85 | 175 | 156 |
| | | AGTG | 498 | 532 | 851 | 1476 | 1209 | 2196 | 1092 |
| | | AGTT | 378 | 48 | 33 | 61 | 40 | 68 | 56 |
| | | ATAA | 183 | 428 | 319 | 426 | 353 | 915 | 583 |
| | | ATAA | 225 | 17 | 40 | 39 | 49 | 128 | 82 |
| | | ATAG | 94 | 52 | 98 | 63 | 343 | 469 | 76 |
| | | ATAG | 108 | 1111 | 995 | 933 | 833 | 713 | 869 |
| | | ATAG | 402 | 495 | 416 | 472 | 546 | 535 | 482 |
| | | ATAT | 140 | 37 | 20 | 44 | 53 | 45 | 57 |
| | | ATCA | 90 | 423 | 666 | 451 | 172 | 379 | 180 |
| | | ATCA | 199 | 774 | 588 | 493 | 335 | 336 | 352 |
| | | ATCT | 99 | 59 | 43 | 56 | 35 | 125 | 67 |
| | | ATCT | 392 | 139 | 176 | 287 | 262 | 569 | 226 |
| | | ATGA | 162 | 91 | 95 | 127 | 239 | 191 | 262 |
| | | ATGC | 78 | 138 | 91 | 111 | 190 | 466 | 148 |
| | | ATGC | 124 | 317 | 884 | 743 | 403 | 164 | 317 |
| | | ATGC | 236 | 15 | 23 | 76 | 7 | 54 | 119 |
| | | ATGC | 344 | 153 | 108 | 131 | 187 | 217 | 185 |
| | | ATGG | 96 | 118 | 231 | 173 | 115 | 113 | 305 |
| | | ATGG | 365 | 15 | 26 | 22 | 25 | 63 | 29 |
| | | ATGT | 378 | 28 | 47 | 90 | 54 | 108 | 80 |
| | | ATGT | 383 | 26 | 61 | 78 | 40 | 136 | 63 |
| | | ATTA | 256 | 36 | 29 | 27 | 46 | 61 | 81 |
| | | ATTA | 259 | 48 | 54 | 55 | 65 | 75 | 106 |
| | | ATTG | 88 | 100 | 147 | 147 | 262 | 318 | 114 |
| | | ATTG | 485 | 22 | 27 | 27 | 26 | 100 | 29 |
| | | ATTT | 186 | 87 | 60 | 58 | 64 | 190 | 122 |
| | | ATTT | 189 | 99 | 79 | 74 | 85 | 209 | 127 |
| | | ATTT | 313 | 79 | 49 | 94 | 86 | 511 | 197 |
| | | ATTT | 499 | 62 | 80 | 78 | 61 | 265 | 114 |
| | | CAAA | 423 | 398 | 255 | 395 | 302 | 506 | 434 |
| | | CAAC | 471 | 87 | 67 | 99 | 85 | 134 | 104 |
| | | CAAC | 474 | 93 | 77 | 109 | 85 | 151 | 128 |
| | | CAAT | 319 | 23 | 18 | 22 | 16 | 66 | 30 |
| | | CACA | 253 | 771 | 716 | 598 | 626 | 684 | 579 |
| | | CACA | 348 | 847 | 303 | 241 | 181 | 316 | 342 |
| | | CACA | 374 | 205 | 116 | 308 | 211 | 262 | 175 |
| | | CACC | 98 | 241 | 553 | 402 | 143 | 68 | 363 |
| 2 | CLZ_5 | CACC | 201 | 382 | 653 | 727 | 782 | 775 | 903 |
| 14 | CLZ_6 | CACT | 169 | 1576 | 1400 | 727 | 987 | 933 | 909 |
| | | CAGA | 119 | 388 | 129 | 217 | 102 | 115 | 119 |
| 44 | CLZ_52 | CAGA | 146 | 737 | 728 | 643 | 511 | 354 | 332 |
| | | CAGA | 157 | 927 | 820 | 422 | 943 | 533 | 893 |
| | | CAGA | 214 | 118 | 94 | 79 | 129 | 229 | 163 |
| | | CAGC | 247 | 508 | 1511 | 557 | 483 | 531 | 527 |
| | | CAGG | 129 | 647 | 536 | 588 | 592 | 571 | 493 |
| | | CATA | 172 | 534 | 482 | 447 | 494 | 863 | 625 |
| 3 | CLZ_8 | CATC | 98 | 94 | 333 | 253 | 141 | 76 | 212 |
| | | CATC | 135 | 350 | 483 | 606 | 403 | 299 | 464 |
| | | CATG | 78 | 78 | 58 | 56 | 98 | 126 | 217 |
| | | CATG | 197 | 406 | 401 | 421 | 474 | 427 | 318 |
| 43 | CLZ_51 | CATG | 247 | 1740 | 1436 | 2195 | 3089 | 2713 | 4020 |
| | | CATT | 420 | 194 | 114 | 155 | 122 | 259 | 214 |
| | | CATT | 429 | 119 | 89 | 96 | 105 | 198 | 141 |
| | | CATT | 432 | 127 | 101 | 106 | 104 | 229 | 157 |
| | | CCAC | 404 | 28 | 12 | 23 | 37 | 51 | 93 |
| | | CCAG | 87 | 58 | 29 | 28 | 115 | 100 | 229 |
| 4 | CLZ_10 | CCAG | 104 | 211 | 309 | 353 | 154 | 153 | 262 |
| | | CCAT | 119 | 122 | 38 | 91 | 35 | 113 | 179 |
| | | CCAT | 133 | 57 | 45 | 66 | 59 | 95 | 100 |
| | | CCAT | 296 | 16 | 34 | 7 | 8 | 80 | 56 |
| | | CCAT | 440 | 56 | 76 | 86 | 104 | 83 | 97 |
| | | CCCC | 123 | 474 | 860 | 910 | 628 | 277 | 698 |
| | | CCCG | 243 | 163 | 654 | 354 | 120 | 146 | 129 |
| | | CCCG | 277 | 218 | 282 | 257 | 310 | 660 | 337 |
| | | CCCG | 283 | 298 | 261 | 421 | 250 | 779 | 323 |
| | | CCCG | 454 | 84 | 69 | 115 | 90 | 140 | 102 |
| | | CCCT | 119 | 107 | 76 | 104 | 146 | 176 | 132 |
| | | CCGC | 88 | 32 | 231 | 134 | 82 | 843 | 226 |
| | | CCGC | 93 | 197 | 52 | 18 | 743 | 462 | 367 |
| | | CCGC | 118 | 2960 | 2515 | 1919 | 1789 | 1038 | 540 |
| | | CCGC | 309 | 153 | 126 | 94 | 78 | 164 | 156 |
| | | CCGG | 89 | 201 | 406 | 535 | 612 | 446 | 377 |
| 40 | CLZ_48 | CCGG | 94 | 176 | 705 | 527 | 578 | 482 | 702 |
| | | CCGG | 249 | 563 | 188 | 384 | 393 | 295 | 487 |
| | | CCGG | 263 | 535 | 275 | 183 | 219 | 309 | 161 |
| | | CCGT | 169 | 363 | 246 | 408 | 247 | 559 | 398 |
| 5 | CLZ_12 | CCGT | 172 | 765 | 511 | 343 | 347 | 407 | 174 |
| 42 | CLZ_50 | CCGT | 293 | 88 | 57 | 65 | 52 | 426 | 251 |
| | | CCGT | 350 | 82 | 24 | 91 | 37 | 52 | 100 |
| | | CCTA | 110 | 174 | 342 | 363 | 204 | 214 | 195 |
| | | CCTA | 379 | 80 | 89 | 170 | 105 | 192 | 217 |
| | | CCTC | 382 | 72 | 83 | 88 | 66 | 105 | 110 |
| | | CCTG | 99 | 283 | 93 | 245 | 1081 | 319 | 379 |
| | | CCTG | 130 | 1413 | 1995 | 1550 | 934 | 1004 | 1180 |
| | | CCTT | 104 | 304 | 533 | 768 | 344 | 288 | 0 |
| | | CGAA | 101 | 66 | 225 | 382 | 71 | 130 | 305 |
| | | CGAC | 76 | 71 | 45 | 704 | 87 | 174 | 1047 |
| | | CGAC | 148 | 1008 | 1239 | 1016 | 884 | 1043 | 999 |
| | | CGAC | 480 | 556 | 498 | 421 | 605 | 1183 | 913 |
| | | CGAC | 490 | 317 | 250 | 225 | 282 | 531 | 473 |
| | | CGAG | 273 | 212 | 98 | 136 | 89 | 96 | 136 |
| | | CGAG | 450 | 122 | 122 | 101 | 173 | 230 | 181 |
| | | CGAT | 78 | 322 | 85 | 178 | 293 | 484 | 420 |
| | | CGAT | 95 | 42 | 40 | 62 | 80 | 94 | 50 |
| | | CGAT | 98 | 48 | 62 | 67 | 68 | 124 | 52 |
| | | CGAT | 105 | 97 | 59 | 45 | 199 | 206 | 151 |
| | | CGAT | 268 | 770 | 202 | 374 | 593 | 519 | 478 |
| | | CGAT | 496 | 170 | 164 | 127 | 196 | 147 | 146 |
| | | CGCA | 88 | 592 | 249 | 355 | 696 | 542 | 854 |
| | | CGCA | 334 | 1071 | 1923 | 1725 | 1333 | 1445 | 1438 |
| | | CGCA | 472 | 218 | 306 | 294 | 365 | 312 | 406 |
| | | CGCG | 82 | 61 | 115 | 148 | 377 | 254 | 133 |
| | | CGCG | 85 | 32 | 115 | 60 | 275 | 248 | 133 |
| | | CGCG | 111 | 49 | 236 | 266 | 826 | 778 | 323 |
| | | CGCG | 371 | 27 | 37 | 72 | 44 | 101 | 56 |
| | | CGCT | 118 | 905 | 634 | 948 | 855 | 668 | 542 |
| | | CGCT | 341 | 22 | 29 | 39 | 11 | 62 | 23 |
| | | CGGC | 87 | 66 | 89 | 149 | 216 | 198 | 150 |
| | | CGGC | 110 | 311 | 620 | 1099 | 292 | 124 | 687 |
| | | CGGG | 85 | 259 | 928 | 777 | 314 | 252 | 437 |
| | | CGGG | 102 | 35 | 35 | 175 | 93 | 365 | 99 |
| | | CGGG | 109 | 34 | 28 | 63 | 65 | 112 | 96 |
| | | CGGG | 135 | 100 | 203 | 120 | 91 | 434 | 537 |
| | | CGGG | 402 | 116 | 116 | 170 | 205 | 226 | 178 |
| | | CGGG | 490 | 59 | 69 | 116 | 116 | 142 | 100 |
| | | CGGT | 142 | 207 | 147 | 171 | 201 | 301 | 322 |
| 6 | CLZ_15 | CGGT | 217 | 174 | 116 | 130 | 91 | 87 | 83 |
| | | CGGT | 476 | 46 | 30 | 29 | 41 | 60 | 53 |
| | | CGTC | 342 | 71 | 87 | 121 | 79 | 393 | 92 |
| | | CGTG | 124 | 346 | 240 | 174 | 115 | 144 | 168 |
| | | CGTG | 234 | 346 | 131 | 129 | 105 | 71 | 119 |
| | | CGTG | 306 | 796 | 1334 | 1296 | 1163 | 1164 | 1114 |
| | | CGTT | 81 | 42 | 91 | 35 | 129 | 186 | 74 |
| | | CGTT | 245 | 169 | 161 | 216 | 168 | 402 | 185 |
| | | CTAA | 268 | 125 | 133 | 121 | 157 | 151 | 201 |
| 37 | CLZ_43 | CTAA | 461 | 120 | 131 | 146 | 185 | 397 | 220 |
| | | CTAC | 93 | 90 | 73 | 124 | 101 | 146 | 106 |
| | | CTAC | 359 | 184 | 161 | 249 | 238 | 357 | 258 |
| | | CTAG | 91 | 48 | 29 | 64 | 113 | 142 | 175 |
| | | CTAG | 97 | 360 | 331 | 395 | 116 | 102 | 537 |
| 15 | CLZ_16 | CTAG | 171 | 412 | 247 | 167 | 119 | 181 | 142 |
| | | CTAT | 190 | 61 | 41 | 67 | 59 | 89 | 74 |
| 28 | CLZ_17 | CTCA | 206 | 567 | 522 | 466 | 306 | 370 | 239 |
| | | CTCA | 313 | 39 | 19 | 47 | 36 | 51 | 55 |
| | | CTCG | 140 | 90 | 94 | 293 | 259 | 663 | 605 |
| | | CTCG | 218 | 1262 | 450 | 734 | 340 | 124 | 208 |
| | | CTCG | 331 | 59 | 28 | 84 | 49 | 88 | 104 |
| | | CTCG | 490 | 352 | 257 | 320 | 376 | 616 | 504 |
| | | CTCG | 498 | 258 | 152 | 234 | 315 | 597 | 488 |
| | | CTCT | 137 | 503 | 422 | 462 | 762 | 965 | 828 |
| | | CTCT | 142 | 1146 | 797 | 1258 | 1620 | 1881 | 1685 |
| | | CTGA | 115 | 29 | 30 | 42 | 30 | 130 | 55 |
| 41 | CLZ_49 | CTGA | 450 | 127 | 173 | 228 | 279 | 258 | 265 |
| | | CTGC | 116 | 0 | 449 | 479 | 212 | 188 | 0 |
| 36 | CLZ_18 | CTGC | 320 | 0 | 60 | 83 | 99 | 104 | 0 |
| | | CTGG | 84 | 102 | 54 | 62 | 90 | 117 | 126 |
| | | CTGG | 183 | 269 | 195 | 328 | 321 | 308 | 1166 |
| | | CTTA | 86 | 49 | 24 | 69 | 48 | 73 | 52 |
| | | CTTA | 132 | 58 | 45 | 58 | 60 | 97 | 58 |
| | | CTTA | 378 | 297 | 350 | 416 | 443 | 747 | 450 |
| | | CTTA | 494 | 31 | 24 | 39 | 24 | 56 | 44 |
| | | CTTA | 499 | 10 | 29 | 45 | 42 | 69 | 52 |
| | | CTTC | 77 | 26 | 30 | 49 | 58 | 64 | 45 |
| | | CTTG | 83 | 792 | 397 | 700 | 601 | 967 | 1173 |
| | | CTTG | 176 | 119 | 75 | 200 | 187 | 192 | 229 |
| | | GAAC | 78 | 35 | 17 | 117 | 36 | 36 | 51 |
| | | GAAG | 93 | 122 | 348 | 230 | 116 | 116 | 183 |
| | | GAAG | 148 | 552 | 569 | 635 | 454 | 343 | 560 |
| | | GAAG | 196 | 363 | 237 | 448 | 447 | 223 | 350 |
| | | GAAG | 223 | 44 | 31 | 51 | 63 | 71 | 101 |
| | | GAAG | 226 | 44 | 31 | 51 | 62 | 71 | 81 |
| | | GAAG | 231 | 18 | 15 | 30 | 31 | 71 | 85 |
| | | GACG | 79 | 26 | 20 | 38 | 47 | 57 | 62 |
| | | GACG | 97 | 597 | 409 | 195 | 127 | 214 | 160 |
| | | GACG | 423 | 187 | 294 | 260 | 280 | 377 | 377 |
| | | GACT | 155 | 117 | 111 | 137 | 201 | 241 | 147 |
| | | GAGG | 103 | 136 | 175 | 399 | 79 | 90 | 139 |
| | | GAGG | 248 | 227 | 82 | 85 | 120 | 112 | 117 |
| | | GAGT | 367 | 302 | 382 | 345 | 369 | 355 | 326 |
| | | GATA | 345 | 15 | 33 | 31 | 50 | 94 | 30 |
| | | GATC | 95 | 81 | 170 | 177 | 112 | 67 | 130 |
| 31 | CLZ_58 | GATC | 258 | 60 | 177 | 141 | 125 | 169 | 142 |
| | | GATC | 356 | 34 | 35 | 67 | 48 | 108 | 42 |
| | | GATG | 300 | 375 | 310 | 202 | 280 | 270 | 293 |
| | | GATT | 91 | 50 | 18 | 32 | 41 | 40 | 55 |
| | | GCAA | 90 | 211 | 210 | 261 | 303 | 206 | 194 |
| | | GCAA | 269 | 222 | 90 | 150 | 140 | 218 | 237 |
| | | GCAC | 92 | 63 | 82 | 119 | 59 | 416 | 266 |
| | | GCAC | 186 | 282 | 238 | 186 | 308 | 203 | 156 |
| | | GCAT | 121 | 229 | 260 | 229 | 149 | 166 | 222 |
| | | GCAT | 439 | 19 | 25 | 28 | 34 | 57 | 35 |
| | | GCCA | 112 | 189 | 312 | 216 | 134 | 102 | 213 |
| | | GCCA | 240 | 49 | 47 | 22 | 27 | 119 | 68 |
| | | GCCC | 79 | 60 | 42 | 40 | 62 | 89 | 101 |
| | | GCCC | 121 | 62 | 42 | 39 | 57 | 96 | 212 |
| | | GCCC | 294 | 695 | 144 | 403 | 428 | 422 | 469 |
| 45 | CLZ_56 | GCCC | 324 | 202 | 648 | 578 | 521 | 512 | 802 |
| | | GCCG | 139 | 57 | 36 | 128 | 115 | 146 | 87 |
| | | GCCG | 144 | 78 | 39 | 71 | 52 | 101 | 139 |
| | | GCCT | 84 | 122 | 68 | 102 | 166 | 150 | 165 |
| | | GCCT | 118 | 403 | 671 | 853 | 366 | 337 | 489 |
| | | GCCT | 126 | 561 | 294 | 305 | 328 | 188 | 246 |
| | | GCGA | 180 | 235 | 1349 | 636 | 733 | 1018 | 1159 |
| | | GCGA | 293 | 1031 | 312 | 375 | 643 | 332 | 335 |
| 46 | CLZ_57 | GCGC | 325 | 35 | 61 | 60 | 75 | 104 | 95 |
| | | GCGG | 77 | 65 | 79 | 91 | 73 | 193 | 78 |
| | | GCGG | 127 | 51 | 50 | 52 | 107 | 161 | 130 |
| | | GCGG | 254 | 413 | 167 | 190 | 231 | 214 | 251 |
| | | GCGG | 269 | 842 | 133 | 372 | 326 | 480 | 586 |
| | | GCGG | 471 | 93 | 130 | 112 | 129 | 149 | 147 |
| | | GCGT | 140 | 117 | 55 | 78 | 115 | 189 | 159 |
| | | GCGT | 168 | 701 | 465 | 504 | 599 | 429 | 405 |
| | | GCGT | 309 | 498 | 282 | 77 | 186 | 71 | 139 |
| | | GCTA | 109 | 388 | 639 | 619 | 320 | 267 | 550 |
| | | GCTA | 132 | 990 | 829 | 1198 | 735 | 669 | 968 |
| | | GCTA | 223 | 898 | 532 | 586 | 525 | 812 | 522 |
| 16 | CLZ_22 | GCTA | 292 | 444 | 169 | 168 | 171 | 182 | 154 |
| | | GCTC | 174 | 100 | 26 | 34 | 57 | 109 | 114 |
| | | GCTC | 202 | 785 | 866 | 512 | 626 | 949 | 593 |
| | | GCTC | 326 | 752 | 666 | 793 | 862 | 890 | 1479 |
| | | GCTG | 78 | 103 | 116 | 427 | 446 | 587 | 312 |
| | | GCTG | 120 | 1694 | 2136 | 2033 | 1141 | 1119 | 1652 |
| | | GCTG | 172 | 31 | 31 | 116 | 154 | 114 | 74 |
| | | GCTT | 233 | 43 | 20 | 62 | 23 | 51 | 63 |
| | | GGAA | 434 | 49 | 114 | 93 | 142 | 230 | 125 |
| | | GGAC | 231 | 683 | 585 | 478 | 510 | 254 | 236 |
| | | GGAC | 472 | 62 | 50 | 62 | 68 | 112 | 120 |
| | | GGAG | 221 | 423 | 239 | 203 | 217 | 250 | 248 |
| | | GGAG | 372 | 836 | 772 | 775 | 1052 | 913 | 641 |
| | | GGAT | 223 | 1048 | 1430 | 1425 | 1632 | 944 | 1461 |
| | | GGCA | 305 | 155 | 124 | 206 | 194 | 280 | 164 |
| 7 | CLZ_24 | GGCA | 393 | 303 | 544 | 393 | 608 | 725 | 842 |
| | | GGCC | 113 | 334 | 371 | 479 | 204 | 175 | 240 |
| | | GGCC | 134 | 838 | 720 | 633 | 537 | 668 | 608 |
| | | GGCC | 324 | 114 | 115 | 211 | 157 | 238 | 301 |
| | | GGCC | 418 | 40 | 12 | 32 | 28 | 26 | 52 |
| | | GGCG | 113 | 235 | 158 | 129 | 129 | 130 | 101 |
| | | GGCG | 136 | 97 | 61 | 76 | 59 | 125 | 145 |
| | | GGCG | 315 | 292 | 238 | 445 | 464 | 495 | 366 |
| 29 | CLZ_26 | GGCT | 129 | 491 | 544 | 423 | 199 | 169 | 321 |
| 47 | CLZ_60 | GGCT | 169 | 467 | 563 | 335 | 704 | 1233 | 1055 |
| | | GGCT | 176 | 127 | 173 | 164 | 410 | 407 | 230 |
| | | GGGA | 172 | 91 | 97 | 67 | 144 | 112 | 183 |
| | | GGGA | 377 | 307 | 157 | 252 | 269 | 263 | 255 |
| | | GGGC | 214 | 59 | 62 | 85 | 66 | 252 | 255 |
| | | GGGC | 286 | 27 | 23 | 34 | 29 | 60 | 71 |
| | | GGGG | 81 | 670 | 1443 | 1269 | 1095 | 2164 | 1645 |
| | | GGGT | 91 | 63 | 68 | 104 | 267 | 143 | 91 |
| | | GGTA | 128 | 265 | 198 | 142 | 124 | 153 | 146 |
| | | GGTA | 184 | 1209 | 969 | 875 | 1109 | 836 | 941 |
| 30 | CLZ_28 | GGTA | 257 | 1016 | 872 | 549 | 492 | 539 | 422 |
| | | GGTG | 139 | 992 | 884 | 936 | 801 | 733 | 811 |
| | | GGTT | 100 | 12 | 17 | 35 | 32 | 41 | 94 |
| | | GTAA | 257 | 86 | 36 | 105 | 119 | 75 | 98 |
| | | GTAC | 107 | 815 | 975 | 1034 | 821 | 751 | 1057 |
| | | GTAG | 244 | 260 | 237 | 294 | 349 | 736 | 282 |
| | | GTAG | 459 | 113 | 137 | 168 | 239 | 351 | 199 |
| | | GTAG | 459 | 113 | 137 | 168 | 239 | 351 | 199 |
| | | GTAG | 471 | 75 | 68 | 76 | 103 | 172 | 99 |
| | | GTCC | 87 | 448 | 256 | 218 | 325 | 193 | 176 |
| | | GTCC | 124 | 111 | 443 | 155 | 139 | 104 | 160 |
| | | GTCC | 187 | 1253 | 1031 | 1066 | 1018 | 891 | 778 |
| | | GTCC | 413 | 28 | 29 | 42 | 35 | 61 | 42 |
| | | GTCG | 176 | 55 | 58 | 79 | 190 | 130 | 126 |
| | | GTCG | 228 | 3085 | 2559 | 3211 | 3000 | 3470 | 3051 |
| | | GTCT | 84 | 19 | 28 | 30 | 43 | 128 | 35 |
| | | GTGC | 87 | 58 | 106 | 159 | 316 | 867 | 410 |
| | | GTGG | 125 | 1407 | 1734 | 1004 | 1276 | 1047 | 1475 |
| | | GTGG | 147 | 821 | 314 | 343 | 174 | 188 | 188 |
| | | GTGG | 458 | 45 | 22 | 41 | 35 | 26 | 33 |
| | | GTTC | 491 | 90 | 236 | 206 | 175 | 240 | 176 |
| | | GTTG | 93 | 156 | 129 | 90 | 93 | 150 | 88 |
| | | GTTG | 114 | 20 | 37 | 44 | 58 | 75 | 78 |
| | | GTTG | 378 | 66 | 35 | 74 | 59 | 80 | 73 |
| | | GTTT | 260 | 49 | 24 | 33 | 42 | 56 | 49 |
| | | GTTT | 336 | 37 | 42 | 40 | 36 | 139 | 126 |
| | | GTTT | 339 | 31 | 37 | 40 | 34 | 156 | 108 |
| | | GTTT | 495 | 36 | 23 | 34 | 54 | 58 | 50 |
| | | TAAA | 84 | 27 | 25 | 46 | 37 | 60 | 37 |
| | | TAAC | 114 | 38 | 32 | 50 | 48 | 65 | 41 |
| | | TAAC | 222 | 411 | 367 | 454 | 384 | 216 | 229 |
| | | TAAC | 450 | 678 | 538 | 407 | 452 | 753 | 669 |
| | | TAAG | 386 | 210 | 334 | 126 | 421 | 702 | 301 |
| | | TACA | 119 | 42 | 49 | 73 | 98 | 111 | 103 |
| | | TACA | 129 | 282 | 242 | 227 | 197 | 206 | 180 |
| | | TACA | 200 | 801 | 493 | 438 | 442 | 477 | 324 |
| | | TACC | 99 | 132 | 141 | 88 | 51 | 18 | 102 |
| | | TACC | 129 | 185 | 160 | 327 | 486 | 457 | 247 |
| | | TACC | 169 | 122 | 72 | 83 | 103 | 179 | 255 |
| | | TACC | 344 | 88 | 71 | 89 | 79 | 104 | 183 |
| 17 | CLZ_32 | TACG | 274 | 181 | 206 | 160 | 187 | 255 | 578 |
| | | TACT | 151 | 94 | 34 | 53 | 44 | 97 | 132 |
| 8 | CLZ_33 | TACT | 188 | 184 | 278 | 1200 | 581 | 339 | 347 |
| | | TACT | 386 | 36 | 50 | 70 | 56 | 104 | 88 |
| | | TAGA | 125 | 41 | 88 | 152 | 95 | 195 | 106 |
| | | TAGA | 134 | 286 | 263 | 214 | 194 | 146 | 152 |
| | | TAGA | 242 | 32 | 9 | 26 | 37 | 142 | 51 |
| | | TAGC | 186 | 1357 | 1306 | 1263 | 1125 | 959 | 889 |
| | | TAGC | 411 | 56 | 68 | 76 | 76 | 142 | 123 |
| | | TAGC | 415 | 50 | 60 | 40 | 66 | 127 | 87 |
| | | TAGC | 464 | 183 | 184 | 166 | 133 | 129 | 106 |
| | | TAGG | 250 | 461 | 166 | 238 | 189 | 306 | 257 |
| | | TAGT | 81 | 213 | 160 | 178 | 286 | 473 | 369 |
| | | TAGT | 97 | 271 | 144 | 246 | 309 | 537 | 299 |
| | | TATA | 98 | 115 | 183 | 488 | 127 | 99 | 230 |
| | | TATA | 382 | 37 | 36 | 49 | 44 | 113 | 39 |
| 50 | CLZ_65 | TATC | 159 | 434 | 327 | 334 | 404 | 701 | 2760 |
| | | TATC | 262 | 119 | 154 | 204 | 168 | 826 | 154 |
| 49 | CLZ_62 | TATG | 290 | 135 | 103 | 59 | 121 | 37 | 52 |
| | | TATG | 446 | 201 | 229 | 389 | 325 | 462 | 328 |
| 9 | CLZ_34 | TATT | 89 | 156 | 623 | 509 | 129 | 186 | 314 |
| | | TATT | 112 | 50 | 38 | 182 | 101 | 122 | 50 |
| | | TATT | 119 | 43 | 16 | 25 | 52 | 40 | 43 |
| | | TATT | 230 | 403 | 42 | 24 | 31 | 35 | 103 |
| | | TATT | 272 | 59 | 43 | 59 | 57 | 131 | 88 |
| | | TATT | 354 | 44 | 36 | 63 | 42 | 147 | 99 |
| | | TCAA | 447 | 44 | 38 | 39 | 26 | 85 | 49 |
| | | TCAC | 134 | 836 | 1637 | 842 | 57 | 1228 | 1047 |
| | | TCAC | 212 | 777 | 567 | 742 | 688 | 573 | 552 |
| | | TCAC | 289 | 1707 | 1138 | 1116 | 842 | 943 | 1123 |
| | | TCAG | 84 | 56 | 68 | 205 | 125 | 148 | 108 |
| | | TCAT | 88 | 88 | 145 | 178 | 409 | 401 | 430 |
| 18 | CLZ_36 | TCAT | 349 | 2478 | 380 | 1155 | 1425 | 903 | 1832 |
| 48 | CLZ_64 | TCAT | 391 | 314 | 216 | 421 | 391 | 554 | 699 |
| | | TCAT | 473 | 45 | 22 | 38 | 39 | 53 | 47 |
| | | TCCA | 106 | 150 | 71 | 193 | 91 | 179 | 385 |
| | | TCCA | 222 | 400 | 303 | 362 | 613 | 787 | 616 |
| | | TCCA | 435 | 68 | 78 | 56 | 57 | 241 | 71 |
| | | TCCA | 439 | 54 | 78 | 56 | 61 | 174 | 71 |
| 10 | CLZ_37 | TCCC | 97 | 381 | 1687 | 1532 | 720 | 673 | 1083 |
| | | TCCC | 148 | 1050 | 865 | 963 | 700 | 639 | 685 |
| | | TCCG | 120 | 0 | 832 | 774 | 566 | 649 | 653 |
| | | TCCG | 185 | 0 | 311 | 292 | 223 | 206 | 259 |
| | | TCCT | 98 | 577 | 621 | 882 | 925 | 1258 | 1741 |
| | | TCCT | 144 | 492 | 551 | 427 | 580 | 313 | 410 |
| | | TCCT | 166 | 740 | 488 | 588 | 605 | 421 | 473 |
| | | TCCT | 275 | 72 | 20 | 77 | 52 | 108 | 133 |
| | | TCGA | 255 | 533 | 263 | 431 | 473 | 614 | 575 |
| | | TCGA | 370 | 167 | 148 | 178 | 194 | 215 | 229 |
| | | TCGC | 196 | 229 | 155 | 214 | 97 | 412 | 311 |
| | | TCGC | 328 | 465 | 545 | 856 | 482 | 674 | 773 |
| | | TCGT | 326 | 32 | 32 | 95 | 33 | 85 | 34 |
| | | TCTA | 80 | 49 | 65 | 184 | 75 | 563 | 231 |
| | | TCTA | 217 | 39 | 50 | 160 | 325 | 212 | 84 |
| | | TCTC | 143 | 341 | 256 | 203 | 262 | 229 | 141 |
| | | TCTT | 155 | 93 | 80 | 96 | 91 | 252 | 110 |
| | | TGAA | 240 | 542 | 390 | 530 | 667 | 552 | 540 |
| | | TGAC | 193 | 1029 | 566 | 798 | 752 | 902 | 1048 |
| 19 | CLZ_42 | TGAC | 328 | 194 | 216 | 199 | 314 | 475 | 303 |
| | | TGAT | 97 | 45 | 44 | 23 | 72 | 158 | 85 |
| | | TGAT | 138 | 608 | 468 | 542 | 442 | 467 | 498 |
| 11 | CLZ_38 | TGCA | 109 | 339 | 554 | 561 | 473 | 736 | 395 |
| | | TGCA | 185 | 137 | 83 | 67 | 160 | 382 | 346 |
| | | TGCC | 163 | 271 | 347 | 93 | 330 | 958 | 407 |
| | | TGCC | 185 | 1164 | 1680 | 573 | 1081 | 1145 | 992 |
| | | TGCC | 343 | 604 | 628 | 832 | 675 | 889 | 1068 |
| | | TGCG | 77 | 188 | 156 | 495 | 125 | 366 | 403 |
| | | TGCG | 111 | 36 | 50 | 76 | 225 | 167 | 155 |
| | | TGGA | 93 | 173 | 157 | 202 | 253 | 545 | 240 |
| | | TGGA | 108 | 1941 | 294 | 2077 | 1692 | 1853 | 2640 |
| | | TGGA | 154 | 823 | 1504 | 1481 | 1370 | 1122 | 673 |
| | | TGGA | 277 | 50 | 23 | 54 | 56 | 103 | 93 |
| | | TGGA | 308 | 31 | 32 | 52 | 51 | 149 | 84 |
| | | TGGC | 105 | 634 | 538 | 630 | 818 | 1092 | 669 |
| | | TGGC | 113 | 377 | 259 | 371 | 510 | 524 | 415 |
| | | TGGC | 160 | 156 | 213 | 282 | 223 | 460 | 320 |
| | | TGGC | 266 | 468 | 451 | 365 | 280 | 207 | 270 |
| | | TGGC | 276 | 73 | 81 | 59 | 81 | 251 | 274 |
| | | TGGC | 494 | 98 | 43 | 27 | 58 | 88 | 122 |
| | | TGGG | 93 | 33 | 65 | 48 | 55 | 228 | 583 |
| | | TGGG | 271 | 241 | 591 | 580 | 426 | 642 | 607 |
| | | TGGT | 103 | 76 | 25 | 97 | 93 | 132 | 236 |
| | | TGGT | 114 | 339 | 537 | 421 | 221 | 204 | 231 |
| | | TGGT | 122 | 119 | 145 | 180 | 135 | 341 | 182 |
| | | TGGT | 158 | 465 | 286 | 403 | 324 | 267 | 348 |
| | | TGGT | 330 | 666 | 673 | 726 | 770 | 701 | 753 |
| | | TGTA | 121 | 1021 | 1596 | 1727 | 1052 | 696 | 1206 |
| | | TGTA | 169 | 1562 | 681 | 624 | 801 | 880 | 753 |
| | | TGTC | 84 | 160 | 250 | 216 | 410 | 510 | 399 |
| | | TGTC | 109 | 711 | 704 | 686 | 276 | 149 | 466 |
| | | TGTG | 315 | 71 | 56 | 83 | 35 | 125 | 73 |
| | | TGTG | 393 | 430 | 313 | 425 | 528 | 419 | 664 |
| | | TGTG | 450 | 573 | 554 | 698 | 819 | 1166 | 654 |
| | | TGTT | 114 | 335 | 752 | 657 | 875 | 794 | 838 |
| | | TGTT | 119 | 703 | 1167 | 993 | 1666 | 1824 | 1251 |
| | | TGTT | 453 | 138 | 226 | 333 | 307 | 324 | 287 |
| | | TTAA | 88 | 149 | 109 | 181 | 377 | 239 | 326 |
| | | TTAA | 194 | 369 | 115 | 230 | 262 | 391 | 313 |
| | | TTAA | 312 | 335 | 177 | 159 | 199 | 136 | 167 |
| | | TTAC | 174 | 287 | 294 | 137 | 192 | 196 | 180 |
| | | TTAG | 104 | 52 | 51 | 54 | 44 | 112 | 65 |
| | | TTAT | 106 | 41 | 22 | 50 | 232 | 53 | 44 |
| | | TTAT | 338 | 486 | 777 | 852 | 875 | 816 | 884 |
| | | TTCC | 96 | 97 | 140 | 133 | 130 | 370 | 135 |
| | | TTCC | 104 | 51 | 31 | 109 | 67 | 94 | 78 |
| | | TTGA | 117 | 20 | 28 | 34 | 38 | 63 | 60 |
| | | TTGC | 119 | 57 | 52 | 67 | 73 | 117 | 75 |
| | | TTGC | 299 | 151 | 114 | 68 | 60 | 65 | 59 |
| | | TTGG | 209 | 704 | 1160 | 894 | 921 | 857 | 1215 |
| | | TTGG | 466 | 60 | 47 | 46 | 71 | 103 | 68 |
| 12 | CLZ_40 | TTGT | 266 | 200 | 52 | 75 | 82 | 67 | 115 |
| | | TTGT | 302 | 38 | 33 | 72 | 48 | 69 | 79 |
| | | TTGT | 483 | 53 | 87 | 120 | 110 | 140 | 60 |
| | | TTTA | 249 | 174 | 32 | 103 | 46 | 55 | 85 |
| | | TTTC | 85 | 31 | 44 | 34 | 89 | 369 | 100 |
| | | TTTC | 107 | 50 | 37 | 20 | 65 | 91 | 68 |
| | | TTTC | 118 | 633 | 721 | 715 | 303 | 257 | 483 |
| | | TTTC | 153 | 188 | 168 | 113 | 141 | 142 | 270 |
| | | TTTC | 171 | 663 | 642 | 709 | 704 | 801 | 589 |
| | | TTTC | 226 | 26 | 31 | 22 | 63 | 63 | 86 |
| | | TTTC | 277 | 566 | 324 | 375 | 327 | 381 | 278 |

TABLE 2

| | | | | | Nucleotide Homology | |
|---|---|---|---|---|---|---|
| | | Digital | | | | Database |
| Seq ID | | Address | | % | DST nucleotide range | nucleotide |
| No | DST ID | (Msp1) | Database Match (Accession #) | Homology | (bp#) | range (bp#) |
| 1 | CLZ_3 | AGTA 106 | Mus musculus serine protease HTRA mRNA, | 100% | 1-46 | 1965-2010 |
| | | | complete cds (AF172994.1) | | | |
| | | | And | | | |
| | | | Mus musculus insulin-like growth factor | 100% | 1-46 | 1960-2005 |
| | | | binding protein 5 protease (AF179369.1) | | | |
| 2 | CLZ_5 | CACC 201 | Mouse mRNA for apolipoprotein D (X82648) | 99% | 1-149 | 433-581 |
| 3 | CLZ_8 | CATC 98 | (EST) UI-M-AN1-afi-g-11-0-UI.s1 | 95% | 1-48 | 10-57 |
| | | | NIH_BMAP_MBG_N Mus musculus cDNA | | | |
| | | | clone/UI-M-AN1-afi-g-11-0-UI 3′, mRNA | | | |
| | | | sequence (AI846711.1) | | | |
| 4 | CLZ_10 | CCAG 104 | (EST) mf92h11.x1 Soares mouse embryo | 98% | 1-55 | 99-153 |
| | | | NbME13.5 14.5 Mus musculus cDNA clone | | | |
| | | | IMAGE 421797 3′, mRNA sequence [Mus | | | |
| | | | musculus] (AI429767) | | | |
| 5 | CLZ_12 | CCGT 172 | Mus musculus importin alpha Q1 mRNA, | 100% | 8-119 | 2-113 |
| | | | complete cds (AF020771) | | | |
| 6 | CLZ_15 | CGGT 217 | Mus musculus dystroglycan (Dag1) mRNA | 98% | 1-160 | 3012-3170 |
| | | | (U43512) | | | |
| 7 | CLZ_24 | GGCA 393 | (EST) UI-R-C1-1d-g-08-0-UI-.s1 UI-R-C1 | 90% | 1-340 | 20-356 |
| | | | Rattus norvegicus cDNA clone UI-R-C1-1d-g- | | | |
| | | | 08-0-UI 3′, mRNA sequence [Rattus | | | |
| | | | norvegicus] (AI502824) | | | |
| 8 | CLZ_33 | TACT 188 | (EST) Mus musculus C57BL/6J 10-day embryo | 95% | 1-131 | 74-204 |
| | | | Mus musculus cDNA clone 2610203G07, | | | |
| | | | mRNA sequence (AV117493.1) | | | |
| 9 | CLZ_34 | TATT 89 | Rattus norvegicus Sprague-Dawley N-methyl- | 100% | 1-32 | 4039-4070 |
| | | | D-aspartate receptor NMDAR1-2a subunit | | | |
| | | | (NMDAR1) mRNA, complete cds (U08262) | | | |
| 10 | CLZ_37 | TCCC 97 | (EST) UI-M-AH1-agt-h-06-0-UI.s1 NIH- | 100% | 1-45 | 13-57 |
| | | | BMAP-MCE-N Mus musculus cDNA clone UI- | | | |
| | | | M-AH1-agt-h-06-0-UI 3′, mRNA sequence | | | |
| | | | (AI849537.1) | | | |
| 11 | CLZ_38 | TGCA 109 | Mus musculus oligodendrocyte-specific protein | 100% | 1-48 | 1745-1792 |
| | | | mRNA, complete cds (U19582)/(AR009501) | | | |
| | | | Sequence 1 from patent U.S. Pat. No. 5756300 | | | |
| 12 | CLZ_40 | TTGT 266 | (EST) vx01g05.x1 Soares 2NbMT Mus | 99% | 1-205 | 4-208 |
| | | | musculus cDNA clone IM mRNA (AI549943.1) | | | |
| 14 | CLZ_6 | CACT 169 | Mus musculus LIM-kinase1 (Limk1) gene, | 86% | 3-118 | 7957-8072 |
| | | | complete cds; Wbscr1 (Wbscr1) gene, | | | |
| | | | alternative splice products, complete cds; and | | | |
| | | | replication factor C, 40 kDa subunit (Rfc2) gene, | | | |
| | | | complete cds (AF139987.1) | | | |
| 15 | CLZ_16 | CTAG 171 | Mus musculus arm-repeat protein | 99% | 1-119 | 2845-2963 |
| | | | NPRAP/neurojungin (Nprap) mRNA | | | |
| | | | (U90331.1) | | | |
| 16 | CLZ_22 | GCTA 292 | (EST) vk75e05.s1 Knowles Solter mouse 2 cell | 99% | 5-211 | 209-415 |
| | | | Mus musculus 960512 5′ (AA549416) | | | |
| 17 | CLZ_32 | TACG 274 | Mus musculus high mobility group protein I-C | 95% | 89-152 | 1-65 |
| | | | gene, exon 5 (L41622) | | | |
| | | | And | | | |
| | | | Mus musculus early blastocyst cDNA, clone | 97% | 1-215 | 245-459 |
| | | | 01B00056NM07 (C89064) | | | |
| 18 | CLZ_36 | TCAT 349 | Homology to rat mRNA for mitochondrial | 94% | 1-298 | 397-694 |
| | | | enoyl-CoA hydratase (EC4.2.1.17) (X15958) | | | |
| 19 | CLZ_42 | TGAC 328 | (EST) UI-M-AN1-afc-b-05-0-UI.s1 Mus | 98% | 1-271 | 20-290 |
| | | | musculus cDNA clone (AI843761.1) | | | |
| 36 | CLZ_18 | CTGC 320 | (EST) mj75b02.r1 Soares mouse p3NMF19.5 | 97% | 1-271 | 91-361 |
| | | | Mus musculus cDNA clone 481899 5′ | | | |
| | | | (AA059879) | | | |
| 37 | CLZ_43 | CTAA 461 | (EST) ud25c08.r1 Soares_thymus_2NbMT Mus | 99% | 1-396 | 1-396 |
| | | | musculus cDNA clone (AI158519.1) | | | |
| 38 | CLZ_44 | ACGG 352 | (EST) uj37f10.x1 Sugano mouse kidney mkia | 98% | 1-298 | 84-381 |
| | | | Mus musculus cDNA clone IMAGE: 1922155 3′ | | | |
| | | | similar to TR: Q14120 Q14120 DBP-5 | | | |
| | | | NUCLEAR PROTEIN, mRNA sequence [Mus | | | |
| | | | musculus] (AI315677) | | | |
| 39 | CLZ_47 | AATA 136 | Homology to Homo sapiens Bc1-2 associated | 96% | 1-81 | 1279-1359 |
| | | | transcription factor short form mRNA, | | | |
| | | | complete cds. (AF249273.1) | | | |
| 40 | CLZ_48 | CCGG 94 | (EST) UI-M-BH3-arb-e-09-0-UI.s1 | 97% | 1-42 | 13-54 |
| | | | NIH_BMAP_M_S4 Mus musculus cDNA clone | | | |
| | | | (AW457685.1) | | | |
| 41 | CLZ_49 | CTGA 450 | Mus musculus autoantigen La (SS-B) mRNA, | 100% | 1-397 | 318-714 |
| | | | complete cds (L00993.1) | | | |
| 42 | CLZ_50 | CCGT 293 | (EST) uj28f11.xi Sugano mouse kidney mkia | 97% | 7-237 | 96-326 |
| | | | Mus musculus cDNA clone IMAGE: 1921293 3′ | | | |
| | | | (AI315041.1) | | | |
| 43 | CLZ_51 | CATG 247 | (EST) vm08c06.r1 Knowles Solter mouse | 100% | 1-190 | 5-194 |
| | | | blastocyst B1 Mus musculus cDNA clone | | | |
| | | | IMAGE: 989578 (AA571556.1) | | | |
| 44 | CLZ_52 | CAGA 146 | (EST) NCI_CGAP_Mam1 Mus musculus | 100% | 1-92 | 1-92 |
| | | | cDNA clone (BE914502) | | | |
| 45 | CLZ_56 | GCCC 324 | Homology to R. rattus (Sprague-Dawley) | 93% | 1-265 | 46-310 |
| | | | mRNA for brain myosin II isoform (810 bp) | | | |
| | | | (Z32518.1) | | | |
| 46 | CLZ_57 | GCGC 325 | (EST) G0109H07-3 Mouse E7.5 Embryonic | 99% | 1-268 | 305-572 |
| | | | Portion cDNA Library Mus musculus cDNA | | | |
| | | | clone G0109H07 (AW536880.1) | | | |
| 47 | CLZ_60 | GGCT 169 | Homology to (EST) UI-R-C2-mv-g-10-0-UI.s1 | 95% | 3-103 | 29-125 |
| | | | UI-R-C2 Rattus norvegicus cDNA clone UI-R- | | | |
| | | | C2-mv-g-10-0-UI (AI070642.1) | | | |
| 49 | CLZ_62 | TATG 290 | (EST) vp20e08.r1 | 99% | 1-219 | 10-228 |
| | | | Soares_mammary_gland_NbMMG Mus | | | |
| | | | musculus cDNA clone IMAGE: 1069190 5′ | | | |
| | | | similar to SW: YBF5_YEAST P34220 | | | |
| | | | HYPOTHETICAL 47.4 KD PROTEIN IN | | | |
| | | | PTC3-SEC17 INTERGENIC REGION | | | |
| | | | (AA792913.1) | | | |
| 48 | CLZ_64 | TCAT 391 | Homology to rat mRNA for mitochondrial | 92% | 1-338 | 397-734 |
| | | | enoyl-CoA hydratase (EC 4.2.1.17) (X15958) | | | |
| | | | And | | | |
| | | | (EST) mx28c10.r1 Soares mouse NML Mus | 96% | 1-334 | 130-463 |
| | | | musculus cDNA clone 681522 5′similar to | | | |
| | | | SW: ECHM_RAT P14604 ENOYL-COA | | | |
| | | | HYDRATASE, MITOCHONDRIAL | | | |
| | | | PRECURSOR (AA237635) | | | |
| 50 | CLZ_65 | TATC 159 | Mus musculus Purkinje cell protein 4 (Pcp4), | 99% | 2-102 | 374-474 |
| | | | mRNA (NM_008791.1) | | | |

EST = Expressed Sequence Tag,
N/A = Not Applicable

TABLE 3

| | | Digital | |
|---|---|---|---|
| | | Address | |
| Seq ID No | DST ID | (Msp1) | Gene Identity (Accession #) |
| 28 | CLZ_17 | CTCA 206 | Consensus sequence based on Computer Assembled ESTs: |
| | | | Soares mouse p3NMF19.5 Mus musculus cDNA clone IMAGE: 350746 3′, mRNA sequence (AI415388) |
| | | | UI-M-AM0-ado-e-04-0-UI.s1 NIH_BMAP_MAM Mus musculus cDNA clone UI-M-AM0-ado-e-04-0-UI 3′, |
| | | | mRNA sequence (AI841003) |
| | | | Soares mouse embryo NbME13.5 14.5 Mus musculus cDNA IMAGE: 356159 3′, mRNA sequence (AI413353) |
| | | | Soares mouse embryo NbME13.5 14.5 Mus musculus cDNA IMAGE: 426077 3′, mRNA sequence (AI425991) |
| 29 | CLZ_26 | GGCT 129 | Mus musculus metalloprotease-disintegrin MDC15 mRNA, complete cds (AF006196) |
| 30 | CLZ_28 | GGTA 257 | Consensus sequence based on Computer Assembled ESTs: |
| | | | Mus musculus fertilized egg cDNA 3′-end sequence, clone J0229E09 3′, mRNA sequence (C86593) |
| | | | Life Tech mouse embryo 13 5dpc 10666014 Mus musculus cDNA clone IMAGE: 553802 3′, mRNA sequence |
| | | | (AI428410) |
| | | | Stratagene mouse skin (#937313) Mus musculus cDNA clone IMAGE: 1227449 3′, mRNA sequence (AI561814) |
| 31 | CLZ_58 | GATC 258 | Mus musculus OG-12a homeodomain protein (OG-12) mRNA (U66918) |

TABLE 4A

| | | Digital | | | | | | | | |
|---|---|---|---|---|---|---|---|---|---|---|
| Seq ID | | Address | Database Match | Relative DST Amount | | | | | | Validation |
| No | DST ID | (Msp1) | (Accession #) | Control | 45 Minutes | 7 Hour | 5 Day | 12 Day | 14 Day | Method |
| 12 | CLZ_40 | TTGT 266 | (EST) VX01g05X1 Soares | 100 | 18.2 | 29.3 | 24.3 | 43.4 | 41.4 | Northern |
| | | | 2NbMT Mus musculus cDNA | | | | | | | |
| | | | clone IM mRNA | | | | | | | |
| | | | (AI549943.1) | | | | | | | |

TABLE 4B

| | | Digital | | | Nucleotide homology | |
|---|---|---|---|---|---|---|
| Seq ID | | Address for | | | Extended Seq. | Database nucleotide |
| No | Description | DST (Msp1) | Database Match (Accession #) | % Homology | nucleotide range (bp#) | range (bp#) |
| 13 | CLZ_40 | TTGT 266 | Soares 2NbMT Mus musculus cDNA | 98% | 180-682 | 1-503 |
| | Extended | | clone IMAGE: mRNA sequence [Mus | | | |
| | Sequence | | musculus] | | | |
| | | | (AI509550) | | | |

TABLE 5
Real-Time PCR Validation Primers for CLZ

| | | | | | | |
|---|---|---|---|---|---|---|
| Seq | DST ID/ | Digital | | For | | Rev |
| ID | Description of | Address | | Seq | | Seq |
| No | Extended Seq | (Msp1) | Forward Primer | ID | Reverse Primer | ID |

| | | | | | | |
|---|---|---|---|---|---|---|
| 2 | CLZ 5 | CACC 201 | GGA TCC TGG CCA CCG ATT AT | 95 | TGG TGC AGG AGT ACA CGA GG | 96 |
| 12 | CLZ 40 | TTGT 266 | GGT TCA GCA CGT ATC CAA CGT | 97 | TGC TGG ATG GAG ACT GAA CCT | 98 |
| 37 | CLZ 43 | CTAA 46 | AAT GAT GAG CCA CAG AAC CTC A | 99 | AAC ATG CCA AAA GTG GAA ATA AAT T | 100 |
| 79 | Mouse sequence | N/A | CCA ATG GTT AGC GTT CCA AAA | 81 | CTT CTG CTG CCT TGT TGG TTT | 82 |
| | homolog to | | | | | |
| | Human KIAA 1451 | | | | | |

TABLE 6
Demographic data for the schizophrenic and control subjects

| | | Age | Tissue | PMI | | Drug dose | | | Age | Tissue | PMI |
|---|---|---|---|---|---|---|---|---|---|---|---|
| | Sex | (yrs) | pH | (hrs) | DOI | (mg)^{a} | | Sex | (yrs) | pH | (hrs) |
| SCHIZOPHRENIA: | | | | | | | CONTROL: | | | | |
| 1 | M | 23 | 6.40 | 42 | 6 | 1750 | 1 | M | 23 | 6.13 | 36 |
| 2 | M | 38 | 5.52 | 40 | N/A | 160 | 2 | M | 30 | 6.46 | 24 |
| 3 | F | 27 | 5.85 | 41 | 10 | N/A | 3 | F | 21 | 6.03 | 58 |
| 4 | M | 55 | 6.10 | 25 | 33 | 40 | 4 | M | 50 | 6.43 | 69 |
| 5 | F | 36 | 6.28 | 45 | 4 | 160 | 5 | F | 32 | 6.16 | 56 |
| 6 | M | 22 | 6.29 | 49 | 20 | 2920 | 6 | M | 22 | 6.58 | 51 |
| 7 | M | 36 | 6.04 | 38 | 12 | 200 | 7 | M | 38 | 6.42 | N/A |
| 8 | M | 44 | 6.28 | 32 | 23 | 600 | 8 | M | 43 | 6.25 | 45 |
| 9 | M | 48 | 6.62 | 30 | 24 | 1250 | 9 | M | 25 | 6.15 | 35 |
| 10 | M | 42 | 6.26 | 47 | 22 | N/A | 10 | M | 42 | 6.32 | 26 |
| 11 | M | 25 | 6.38 | 49 | 2 | N/A | 11 | M | 42 | 6.32 | 26 |
| 12 | M | 22 | 6.07 | 37 | 3 | 450 | 12 | M | 25 | 6.48 | 50 |
| 13 | F | 35 | 6.26 | 15 | 7 | 300 | 13 | M | 26 | 6.42 | 24 |
| 14 | M | 41 | 6.20 | 31 | 11 | 500 | 14 | M | 30 | 5.86 | 27 |
| 15 | M | 45 | 6.48 | 68 | 12 | 300 | 15 | M | 38 | 6.19 | 44 |
| 16 | M | 38 | 6.02 | 50 | 4 | 500 | 16 | F | 33 | 6.41 | 42 |
| 17 | F | 31 | 6.27 | 27 | 13 | 875 | 17 | M | 42 | 6.61 | 43 |
| 18 | F | 38 | 6.43 | 20 | 17 | N/A | 18 | M | 43 | 6.43 | 51 |
| 19 | M | 22 | 6.17 | 37 | 3 | 200 | 19 | F | 38 | 6.26 | 52 |
| 20 | M | 26 | 6.39 | 52 | 2 | 500 | | | | | |

| | | | | | | | |
|---|---|---|---|---|---|---|---|
| Mean ± | 34.7 ± 2.2 | 6.21 ± 0.05 | 38.7 ± 2.9 | Mean ± | 33.2 ± 2.2 | 6.32 ± 0.04 | 41.5 ± 4.5 |
| SEM | | | | SEM | | | |

PMI, post-mortem interval;
DOI, duration of illness;
N/A, not available;
^{a}drug doses are given as chlorpromazine equivalents.

TABLE 7
Demographic data for the bipolar and control subjects

| | | | | | |
|---|---|---|---|---|---|
| | Age | Tissue | PMI | | Neuroleptic |
| Sex | (years) | pH | (hrs) | DOI | Drugs |

BIPOLAR:

| | | | | | | |
|---|---|---|---|---|---|---|
| 1 | F | 74 | 6.26 | 45 | 12 | Fluphenazine |
| 2 | F | 58 | 5.68 | 41 | 40 | none |
| 3 | M | 59 | 6.46 | 34 | 24 | none |
| 4 | M | 38 | 6.42 | 24 | 10 | Chlorpromazine |
| 5 | M | 66 | 6.41 | 17 | 3 | Fluphenazine |
| 6 | F | 55 | 6.46 | 52 | 14 | Trifluorperazine |
| 7 | F | 60 | 6.08 | 50 | 23 | Flupenthixol |
| 8 | M | 61 | 6.44 | 58 | 35 | Melleril |

| | | | |
|---|---|---|---|
| Mean ± | 58.8 ± | 6.27 ± 0.09 | 40.1 ± 5.0 |
| SEM | 3.6** | | |

CONTROL:

| | | | | |
|---|---|---|---|---|
| 1 | F | 38 | 6.26 | 52 |
| 2 | F | 33 | 6.41 | 42 |
| 3 | M | 38 | 6.19 | 44 |
| 4 | M | 30 | 6.42 | N/A |
| 5 | M | 26 | 6.42 | 24 |
| 6 | M | 43 | 6.25 | 45 |
| 7 | F | 32 | 6.16 | 56 |
| 8 | M | 42 | 6.61 | 43 |

| | | | |
|---|---|---|---|
| Mean ± | 35.3 ± | 6.34 ± 0.05 | 43.7 ± 3.8 |
| SEM | 2.1 | | |

**p < 0.0001

TABLE 8
ApoD Protein Levels in Various Brain Regions from Normal and
Schizophrenic Subjects

| | ApoD (μg/mg protein) | |
|---|---|---|
| Brain Region | Control | Schizophrenic |
| Cortex: | | |
| Lateral PFC | 0.096 ± 0.009 (n = 10) | 0.143 ± 0.015 (n = 10)* |
| Dorso-lateral PFC | 0.127 ± 0.008 (n = 19) | 0.244 ± 0.027 (n = 20)*** |
| Parietal | 0.086 ± 0.007 (n = 10) | 0.111 ± 0.014 (n = 10) |
| Cingulate | 0.048 ± 0.003 (n = 10) | 0.075 ± 0.012 (n = 10) |
| Orbito-Frontal | 0.058 ± 0.002 (n = 10) | 0.072 ± 0.005 (n = 10)* |
| Occipital | 0.196 ± 0.013 (n = 18) | 0.201 ± 0.014 (n = 19) |
| Amygdala | 0.077 ± 0.006 (n = 10) | 0.121 ± 0.016 (n = 10)* |
| Thalamus | 0.266 ± 0.020 (n = 10) | 0.426 ± 0.060 (n = 10)** |
| Caudate | 0.078 ± 0.011 (n = 18) | 0.132 ± 0.021 (n = 20)* |
| S. Nigra | 0.146 ± 0.008 (n = 17) | 0.183 ± 0.019 (n = 17) |
| Hippocampus | 0.059 ± 0.005 (n = 17) | 0.069 ± 0.008 (n = 14) |
| Cerebellum | 0.086 ± 0.009 (n = 18) | 0.088 ± 0.015 (n = 18) |

ApoD concentrations were measured by ELISA using purified apoD as a standard. PFC, prefrontal cortex.
Values are mean concentration ± S.E.M.
Significant differences are indicated by asterisks (student's t test; two-tailed).
***p = 0.0002;
**p = 0.02;
*p < 0.05.

TABLE 9
Summary of Expression Patterns in Mouse CNS

| Clone ID | Identity | Expression Pattern |
|---|---|---|
| CLZ_3 | Serine protease | Cortex, Thalamus, Hippocampus, Striatum, |
| | | Amygdala |
| CLZ_16 | Neurojungin | Ubiquitous |
| CLZ_17 | N-acetylgalactosaminyltransferase | Hypothalamus, Hippocampus |
| CLZ_24 | EST | Ubiquitous, (Cortex-enriched) |
| CLZ_26 | Metalloprotease | Ubiquitous, (Cortex-enriched) |
| CLZ_28 | EST | Ubiquitous, (Cortex-enriched) |
| CLZ_34 | NMDA subunit | Ubiquitous |
| CLZ_38 | Oligodendrocyte-specific protein | White matter regions in the CNS |
| CLZ_40 | EST | Nucleus Accumbens, Dentate Gyrus |
| CLZ_43 | Oxysterol binding protein | Striatum, Cortex |
| CLZ_44 | EST | Cortex, Thalamus, Hippocampus, |
| | | Hypothalamus |
| CLZ_47 | Bc1-2 associated protein | Ubiquitous |
| CLZ_64 | Mitochondrial | Ubiquitous |

We claim:

1. An isolated nucleic acid molecule comprising a polynucleotide chosen from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 associated with psychoses or other neuropsychiatric disorders.

2. An isolated polypeptide encoded by a polynucleotide chosen from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO: 16, SEQ ID NO:17, SEQ ID NO:18, SEQ ID NO:19, SEQ ID NO:28, SEQ ID NO:29, SEQ ID NO:30, SEQ ID NO:31, SEQ ID NO:36, SEQ ID NO:37, SEQ ID NO: 38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:68, SEQ ID NO:79, and SEQ ID NO:80 associated with psychoses or other neuropsychiatric disorders.

3. An isolated nucleic acid molecule comprising a polynucleotide at least 95% identical to the isolated nucleic acid molecule of claim 1.

4. An isolated nucleic acid molecule at least ten bases in length that is hybridizable to the isolated nucleic acid molecule of claim 1 under stringent conditions.

5. An isolated nucleic acid molecule encoding the polypeptide of claim 2.

6. An isolated nucleic acid molecule encoding a fragment of the polypeptide of claim 2.

7. An isolated nucleic acid molecule encoding a polypeptide epitope of the polypeptide of claim 2.

8. The polypeptide of claim 2 wherein the polypeptide has biological activity.

9. An isolated nucleic acid encoding a species homologue of the polypeptide of claim 2.

10. The isolated nucleic acid molecule of claim 1, wherein the nucleotide sequence comprises sequential nucleotide deletions from either the 5′ end or the 3′end.

11. A recombinant vector comprising the isolated nucleic acid molecule of claim 1.

12. A recombinant host cell comprising the isolated nucleic acid molecule of claim 1.

13. A method of making the recombinant host cell of claim 12.

14. The recombinant host cell of claim 12 comprising vector sequences.

15. The isolated polypeptide of claim 2, wherein the isolated polypeptide comprises sequential amino acid deletions from either the C-terminus or the N-terminus.

16. An isolated antibody that binds specifically to the isolated polypeptide of claim 2.

17. The isolated antibody of claim 16 wherein the antibody is a monoclonal antibody.

18. The isolated antibody of claim 16 wherein the antibody is a polyclonal antibody.

19. A recombinant host cell that expresses the isolated polypeptide of claim 2.

20. An isolated polypeptide produced by the steps of:
(a) culturing the recombinant host cell of claim 14 under conditions such that said polypeptide is expressed; and
(b) isolating the polypeptide.

21. A method for preventing, treating, modulating, or ameliorating a medical condition, comprising administering to a mammalian subject a therapeutically effective amount of the polypeptide of claim 2 or the polynucleotide of claim 1.

22. The method of claim 21 wherein the medical condition is a neuropsychiatric disorder.

23. A method for preventing, treating, modulating, or ameliorating neurological disorders comprising administering to a mammalian subject a therapeutically effective amount of the antibody of claim 16.

24. The method of claim 23 wherein the medical condition is a psychoses or other neuropsychiatric disorder.

25. A method of diagnosing a neurological disorder or a susceptibility to a neurological disorder in a subject comprising:
(a) determining the presence or absence of a mutation in the polynucleotide of claim 1; and
(b) diagnosing a pathological condition or a susceptibility to a pathological condition based on the presence or absence of said mutation.

26. The method of claim 25 wherein the pathological condition is a psychoses or other neuropsychiatric disorder.

27. A method of diagnosing a pathological condition or a susceptibility to a neurological condition in a subject comprising detecting an alteration in expression of a polypeptide encoded by the polynucleotide of claim 1, wherein the presence of an alteration in expression of the polypeptide is indicative of the neurological condition or susceptibility to the nuerological condition.

28. The method of claim 27 wherein the alteration in expression is an increase in the amount of expression or a decrease in the amount of expression.

29. The method of claim 27 wherein the pathological condition is a psychoses or other neuropsychiatric disorder.

30. The method of claim 29 wherein the method further comprises the steps of: obtaining a first biological sample from a patient suspected of having a psychoses or other neuropsychiatric disorder and obtaining a second sample from a suitable comparable control source;
(a) determining the amount of at least one polypeptide encoded by a polynucleotide of claim 1 in the first and second sample; and
(b) comparing the amount of the polypeptide in the first and second samples;
wherein a patient is diagnosed as having a psychoses or other neuropsychiatric disorder if the amount of the polypeptide in the first sample is greater than or less than the amount of the polypeptide in the second sample.

31. The use of the polynucleotide of claim 1 or polypeptide of claim 2 for the manufacture of a medicament for the treatment of a psychoses or other neuropsychiatric disorder.

32. The use of the antibody of claim 16 for the manufacture of a medicament for the treatment of a psychoses or other neuropsychiatric disorder.

33. A method for identifying a binding partner to the polypeptide of claim 2 comprising:
(a) contacting the polypeptide of claim 2 with a binding partner; and
(b) determining whether the binding partner effects an activity of the polypeptide.

34. The gene corresponding to the cDNA sequence of the isolated nucleic acid of claim 1.

35. A method of identifying an activity of an expressed polypeptide in a biological assay, wherein the method comprises:
(a) expressing the polypeptide of claim 2 in a cell;
(b) isolating the expressed polypeptide;
(c) testing the expressed polypeptide for an activity in a biological assay; and
(d) identifying the activity of the expressed polypeptide based on the test results.

36. A substantially pure isolated DNA molecule suitable for use as a probe for genes regulated by neuroleptics, chosen from the group consisting of the DNA molecules identified in Table 1, having a 5′ partial nucleotide sequence and length as described by their digital address, and having a characteristic regulation pattern by neuroleptics.

37. A kit for detecting the presence of the polypeptide of the claim 2 in a mammalian tissue sample comprising a first antibody which immunoreacts with a mammalian protein encoded by a gene corresponding to the polynucleotide of claim 1 or with a polypeptide encoded by the polynucleotide of claim 2 in an amount sufficient for at least one assay and suitable packaging material.

38. A kit of claim 37 further comprising a second antibody that binds to the first antibody.

39. The kit of claim 38 wherein the second antibody is labeled.

40. The kit of claim 39 wherein the label comprises enzymes, radioisotopes, fluorescent compounds, colloidal metals, chemiluminescent compounds, phosphorescent compounds, or bioluminescent compounds.

41. A kit for detecting the presence of a gene encoding an protein comprising a polynucleotide of claim 1, or fragment thereof having at least 10 contiguous bases, in an amount sufficient for at least one assay, and suitable packaging material.

42. A method for detecting the presence of a nucleic acid encoding a protein in a mammalian tissue sample, comprising the steps of:
(a) hybridizing a polynucleotide of claim 1 or fragment thereof having at least 10 contiguous bases, with the nucleic acid of the sample; and
(b) detecting the presence of the hybridization product.

43. A method of diagnosing a psychoses or other neuropsychiatric disorder or a susceptibility to a psychoses or other neuropsychiatric disorder in a subject comprising:
(a) determining the presence or absence of a mutation in apolipoprotein D polynucleotide; and
(b) diagnosing a psychoses or other neuropsychiatric disorder or a susceptibility to a psychoses or other neuropsychiatric disorder based on the presence or absence of said mutation.

44. A method of diagnosing a psychoses or other neuropsychiatric disorder or a susceptibility to a psychoses or other neuropsychiatric disorder in a subject comprising:
(a) determining the presence or amount of expression of apolipoprotein D-polypeptide in a biological sample; and
(b) diagnosing a psychoses or other neuropsychiatric disorder or a susceptibility to a psychoses or other neuropsychiatric disorder based on the presence or amount of expression of the apolipoprotein D polypeptide.

45. The method of claim 43 or 44 wherein the neuropsychiatric disorder is schizophrenia.

46. The method of claim 43 or 44 wherein the neuropsychiatric disorder is bipolar disorder.

47. A method of diagnosing a psychoses or other neuropsychiatric disorder or a susceptibility to a psychoses or other neuropsychiatric disorder in a subject comprising:
(a) determining the presence or absence of a mutation in the polynucleotide or polynucleotide fragment of SEQ ID NO: 2 and
(b) diagnosing a psychoses or other neuropsychiatric disorder or a susceptibility to a neuropsychiatric disorder based on the presence or absence of said mutation.

48. A method of diagnosing a neuropsychiatric disorder or a susceptibility to a psychoses or other neuropsychiatric disorder in a subject comprising:
(a) determining the presence or amount of expression of the polypeptide comprising an amino acid sequence at least 95% identical to a polypeptide fragment of a translation of SEQ ID NO: 2 in a biological sample; and
(b) diagnosing a psychoses or other neuropsychiatric disorder or a susceptibility to a psychoses or other neuropsychiatric disorder based on the presence or amount of expression of the polypeptide.

49. The method of claim 47 or 48 wherein the neuropsychiatric disorder is schizophrenia.

50. The method of claim 47 or 48 wherein the neuropsychiatric disorder is bipolar disorder.
